(19) United States
(12) Patent Application Publication
(10) Pub. No.: US 2007/0255115 A1
(43) Pub. Date: Nov. 1, 2007

(54) Remote diagnostic & treatment system

(76) Inventors: Richard L. Anglin JR., Del Mar, CA (US); Bradley T. Tipler, San Diego, CA (US)

Correspondence Address: Giaccherini, Post Office Box 1146, Carmel Valley, CA, 93924, US

(21) Appl. No.: 11/414,746

(22) Filed: Apr. 27, 2006

(51) Int. Cl. A61B 5/00

(52) U.S. Cl. 600/300

(57) ABSTRACT

Methods and apparatus for providing remote diagnosis and treatment are disclosed. One embodiment of the invention comprises a cellular telephone (16A) that includes a camera (34), a display (40), a speaker (36A), a microphone (36B) and embedded diagnostic and treatment software (18). An alternative embodiment may also include one or more data devices (88) that may be connected to the cellular phone (16A) using a wireless (30A) or wired (30B) connection.

### INTRODUCTION

The title of this Non-Provisional Patent Application is Remote Diagnostic & Treatment System. The Applicants are Richard L. Anglin, Jr., 2115 Heather Lane, Del Mar, Calif. 92014-2244 and Bradley T. Tipler, 4165 Pilon Point, San Diego, Calif. 92130-2205. Mr. Anglin is a Citizen of the United States of America; Mr. Tipler is a Citizen of Canada.

### FIELD OF THE INVENTION

One embodiment of the present invention pertains to methods and apparatus for providing a remote diagnostic and treatment system. More particularly, one embodiment of the invention comprises a cellular telephone which includes a camera, a display, a speaker, a microphone and embedded remote control and diagnostic and treatment software. In an alternative embodiment, the invention may also include a variety of data devices which are connected to the cellular phone over a wired or wireless connection.

### FEDERALLY SPONSORED RESEARCH OR DEVELOPMENT

None.

### BACKGROUND OF THE INVENTION

Some current health service providers operate call centers for telephonic triage and health status monitoring. A patient or potential patient phones the call center using a traditional wired or wireless telephone, and is connected to a health service practitioner nurse, paramedic, medical technician or doctor, who then provides advice.

No currently available product offers doctors, nurses, medical technicians or other health care providers a simple and versatile method and apparatus that will collect data and then provide diagnostic and treatment assistance at virtually any location over a wireless connection.

The development of a system that is able to provide audio, video and data information concerning a patient or potential patient from a remote location and is able to guide a user through a diagnostic and treatment procedure at the remote location would constitute a major technological advance, and would satisfy long felt needs and aspirations in the field of health care.

### SUMMARY OF THE INVENTION

One embodiment of the present invention comprises a cellular telephone that includes a camera, a display, a speaker, a microphone and embedded remote control and diagnostic and treatment software. In an alternative embodiment, the invention may also include a variety of data devices which are connected to the cellular phone over a wired or wireless connection. In one embodiment, an operator at a call center may partially or jointly control the cellular telephone and/or a data device.

An appreciation of the other aims and objectives of the present invention and a more complete and comprehensive understanding of this invention may be obtained by studying the following description of a preferred embodiment, and by referring to the accompanying drawings.

### A BRIEF DESCRIPTION OF THE DRAWINGS

FIG. 1 shows a system for collecting information in a first location and conveying that information to a second location for assessment and evaluation.

FIG. 2 shows an embodiment of a system for collecting information in a first location and conveying that information to a second location that is a call center.

FIG. 3 shows a system for collecting information in a first location and conveying that information to a second location via a network.

FIG. 4 shows a system for undertaking remote triage and health status monitoring, a “virtual visit” of a patient or potential patient by a health service practitioner.

FIG. 5 shows a first preferred embodiment of system for undertaking remote triage and health status monitoring, a “virtual visit” of a patient or potential patient by a health service practitioner in which the remote diagnostic means is a cellular or Personal Communications Service (PCS) wireless phone.

FIG. 6 shows a first preferred embodiment of the remote diagnostic means, which is a cellular or Personal Communications Service (PCS) wireless phone with a camera and embedded software that enables remote function control of the wireless phone, including the camera.

FIG. 7 shows a image on a cellular or Personal Communications Service (PCS) phone being converted into a picture.

FIG. 8 shows a first preferred embodiment of a functional block diagram of the embedded software that enables remote function control of a cellular or Personal Communications Service (PCS) wireless phone.

FIG. 9 shows a first embodiment of a screen of the diagnostic, display and control software application deployed on a diagnostic, display and control means.

FIG. 10 shows a first preferred embodiment of a functional block diagram of the diagnostic, display and control software application deployed on a diagnostic, display and control means.

FIG. 11 shows a screen displayed on a diagnostic, display and control means used by a health service practitioner showing a picture.

FIG. 12 shows the disclosed invention in which one or more data devices are connected to device or terminal.

FIG. 13 shows a preferred embodiment of the disclosed invention in which one or more data devices are connected to a device or terminal via a wireless connection.

FIG. 14 shows a preferred embodiment of the disclosed invention in which one or more data devices are connected to a device or terminal via a wired connection.

FIG. 15 shows the functional block diagram for a connection interface device.

FIG. 16 shows a data device, a digital thermometer.

FIG. 17 shows a first embodiment of a functional block diagram of embedded software that enables remote functional control of data devices for the remote diagnostic means as well as a software application that enables remote functional control of the data devices for the remote diagnostic means.

FIG. 18 shows a pop-up window for the location of the temperature reading on the body.

FIG. 19 shows a screen displayed on a diagnostic, display and control means used by a health service practitioner showing the temperature.

FIG. 20 shows the temperature reading fed to the connection interface device for connection to the remote diagnostic means.

FIG. 21 shows a data device, a stethoscope or high fidelity microphone.

FIG. 22 shows a second embodiment of a functional block diagram of embedded software that enables remote functional control of data devices for the remote diagnostic means as well as a software application that enables remote functional control of the data devices for the remote diagnostic means.

FIG. 23 shows a screen displayed on a diagnostic, display and control means used by a health service practitioner showing the pulse.

FIG. 24 shows a transducer converting the stethoscope sound into electrical signals that are fed to the connection interface device for connection to the remote diagnostic means.

FIG. 25 shows a data device, a scale.

FIG. 26 shows a third embodiment of a functional block diagram of embedded software that enables remote functional control of data devices for the remote diagnostic means as well as a software application that enables remote functional control of the data devices for the remote diagnostic means.

FIG. 27 shows a screen displayed on a diagnostic, display and control means used by a health service practitioner showing the weight.

FIG. 28 shows the weight reading fed to the connection interface device for connection to the remote diagnostic means.

FIG. 29 shows a data device, a blood pressure cuff.

FIG. 30 shows a fourth embodiment of a functional block diagram of embedded software that enables remote functional control of data devices for the remote diagnostic means as well as a software application that enables remote functional control of the data devices for the remote diagnostic means.

FIG. 31 shows a screen displayed on a diagnostic, display and control means used by a health service practitioner showing the blood pressure and pulse readings.

FIG. 32 shows the pulse and blood pressure readings fed to the connection interface device for connection to the remote diagnostic means.

FIG. 33 shows a data device, an oximeter.

FIG. 34 shows a fifth embodiment of a functional block diagram of embedded software that enables remote functional control of data devices for the remote diagnostic means as well as a software application that enables remote functional control of the data devices for the remote diagnostic means.

FIG. 35 shows a screen displayed on a diagnostic, display and control means used by a health service practitioner showing the oximeter and pulse readings.

FIG. 36 shows the pulse and oximeter readings fed to the connection interface device for connection to the remote diagnostic means.

FIG. 37 shows a data device, an electrocardiogram (EKG or ECG).

FIG. 38 shows a sixth embodiment of a functional block diagram of embedded software that enables remote functional control of data devices for the remote diagnostic means as well as a software application that enables remote functional control of the data devices for the remote diagnostic means.

FIG. 39 shows a screen displayed on a diagnostic, display and control means used by a health service practitioner showing the electrocardiogram and pulse readings.

FIG. 40 shows the pulse and electrocardiogram readings fed to the connection interface device for connection to the remote diagnostic means.

FIG. 41 shows a data device, a glucose meter.

FIG. 42 shows a seventh embodiment of a functional block diagram of embedded software that enables remote functional control of data devices for the remote diagnostic means as well as a software application that enables remote functional control of the data devices for the remote diagnostic means.

FIG. 43 shows a screen displayed on a diagnostic, display and control means used by a health service practitioner showing the glucose reading.

FIG. 44 shows the glucose reading fed to the connection interface device for connection to the remote diagnostic means.

FIG. 45 shows a data device, an otoscope.

FIG. 46 shows an eighth embodiment of a functional block diagram of embedded software that enables remote functional control of data devices for the remote diagnostic means as well as a software application that enables remote functional control of the data devices for the remote diagnostic means.

FIG. 47 shows a screen displayed on a diagnostic, display and control means used by a health service practitioner showing the glucose reading.

FIG. 48 shows the otoscope picture fed to the connection interface device for connection to the remote diagnostic means.

FIG. 49 shows a data device, an ultrasound device.

FIG. 50 shows a ninth embodiment of a functional block diagram of embedded software that enables remote functional control of data devices for the remote diagnostic means as well as a software application that enables remote functional control of the data devices for the remote diagnostic means.

FIG. 51 shows a screen displayed on a diagnostic, display and control means used by a health service practitioner showing the glucose reading.

FIG. 52 shows the ultrasound readings fed to the connection interface device for connection to the remote diagnostic means.

FIG. 53 shows a data device, a digital camera.

FIG. 54 shows a data device, a digital video camera.

FIG. 55 shows a tenth embodiment of a functional block diagram of embedded software that enables remote functional control of data devices for the remote diagnostic means as well as a software application that enables remote functional control of the data devices for the remote diagnostic means.

FIG. 56 shows the picture or video from an external camera fed to the connection interface device for connection to the remote diagnostic means.

FIG. 57 shows an external camera observing a patient or potential patient utilizing a remote diagnostic means, including data devices.

FIG. 58 shows an alternative embodiment of the remote diagnostic means as a laptop Personal Computer (PC) equipped with a camera.

FIG. 59 shows an alternative embodiment of the remote diagnostic means as a Personal Computer (PC) equipped with a camera.

FIG. 60 shows an alternative embodiment of the disclosed invention in which the remote diagnostic means is a personal computer, specifically a laptop personal computer.

FIG. 61 shows data devices connected to a personal computer using a Bluetooth, wireless fidelity (WiFi) and/or Ultra Wide Band (UWB) connection.

FIG. 62 shows data devices connected to a personal computer via an Universal Serial Bus (USB) connection.

FIG. 63 shows data devices connected to a personal computer via an Institute of Electrical and Electronics Engineers (IEEE) 1394 FireWire connection.

FIG. 64 shows data devices connected to a cellular or Personal Communications Service (PCS) wireless phone using a Bluetooth, wireless fidelity (WiFi) and/or Ultra Wide Band (UWB) connection.

FIG. 65 shows data devices connected to a cellular or Personal Communications Service (PCS) wireless phone via an Universal Serial Bus (USB) connection.

FIG. 66 shows data devices connected to a cellular or Personal Communications Service (PCS) wireless phone via an Institute of Electrical and Electronics Engineers (IEEE) 1394 FireWire connection.

FIG. 67 shows an embodiment of the remote diagnostic means of the disclosed invention to be deployed in an assisted living environment for elderly persons.

FIG. 68 shows an embodiment of the remote diagnostic means of the disclosed invention that includes a Global Positioning System (GPS) receiver.

FIG. 69 shows a screen displayed on a diagnostic, display and control means used by a health service practitioner showing the Global Positioning System (GPS) location of the patient or potential patient.

FIG. 70 shows the disclosed invention used by a health service practitioner to consult with another health service practitioner.

FIG. 71 shows the disclosed invention in which the health service practitioner uses a personal computer at home as the diagnostic, display and control means.

FIG. 72 shows the disclosed invention in which the health service practitioner uses a cellular or Personal Communications Service (PCS) wireless phone as the diagnostic, display and control means.

FIG. 73 shows the disclosed invention used in a physical rehabilitation or athletic coaching application in a fixed setting.

FIG. 74 shows the disclosed invention used in a physical rehabilitation or athletic coaching application on the move.

FIG. 75 shows a Bluetooth, wireless fidelity (WiFI) and Ultra Wide Band (UWB)-enabled heart rate monitor.

FIG. 76 shows a patient or potential patient in a remote location, here a ship, using a satellite phone as a diagnostic means.

FIG. 77 shows a patient or potential patient in a remote location, here an airplane, using a satellite phone as a diagnostic means.

FIG. 78 shows an embodiment of the systems used by a veterinarian to undertake remote triage and health status monitoring of an animal.

FIG. 79 shows a fire investigator using a chemical sniffer attached to a cellular or Personal Communications Service (PCS) wireless phone to get data about the potential causes of a fire that are transmitted to a laboratory analyst for assessment.

FIG. 80 shows a policeman uses a remote fingerprint device attached to a cellular or Personal Communications Service (PCS) wireless phone to fingerprint a suspect, which fingerprint is transmitted to an analyst for review and matching to fingerprint databases.

FIG. 81 shows an engineer using a soil sampling device attached to a cellular or Personal Communications Service (PCS) wireless phone, characteristics of which are transmitted to an laboratory analyst for assessment.

FIG. 82 shows a fire investigator is using a chemical sniffer attached to a walkie-talkie phone who transmits that data in real time to a fire fighter actively fighting the fire nearby.

FIG. 83 shows using OnStar®, which is available in vehicles from General Motors Corporation (GM), as a remote diagnostic means.

FIG. 84 shows using OnStar®, which is available in vehicles from General Motors Corporation (GM), as a remote diagnostic means with a camera outside the car to observe people outside the car.

FIG. 85 shows a grandparent watching grandchildren playing soccer through a cellular or Personal Communications Service (PCS) wireless phone where the grandparent controls the wireless phone.

FIG. 86 shows data devices connected to a home diagnostic device connected to a standard telephone connected to the Public Switched Telephone Network (PSTN).

FIG. 87 shows a functional schematic of a home diagnostic device.

FIG. 88 shows an embodiment of a purpose-designed diagnostic means.

FIG. 89 shows an embodiment of a purpose-designed diagnostic means with included data storage.

FIG. 90 shows an alternative embodiment of a purpose-designed diagnostic means in which the diagnostic means sits in and is recharged by an embodiment of a home diagnostic device.

FIG. 91 shows an alternative embodiment of a designed diagnostic means with a accessory macro lens.

FIG. 92 shows the steps a patient or potential patient must take to receive remote diagnostic and health status monitoring services.

FIG. 93 shows the steps a patient or potential patient must take to receive remote diagnostic and health status monitoring services using a cellular, Personal Communications Service (PCS) or Wireless Fidelity (WiFi) wireless phone.

FIG. 94 shows a first embodiment of a functional block diagram of an institutional structure or business model under which remote diagnostic and health status monitoring services may be provided.

FIG. 95 shows a second embodiment of a functional block diagram of an institutional structure or business model under which remote diagnostic and health status monitoring services may be provided.

FIG. 96 shows a third embodiment of a functional block diagram of an institutional structure or business model under which remote diagnostic and health status monitoring services may be provided.

FIG. 97 shows a patient or potential patient using a personal computer to visit a website, such as www.InternetDoctor.com, to obtain medical information and/or advice from a health service practitioner.

FIG. 98 depicts a patient or potential patient requesting information about a rash on her hand.

FIG. 99 illustrates a patient or potential patient placing her hand near the camera of the personal computer, which allows the health service practitioner to see the rash.

FIGS. 100 and 101 reveal additional dialog between a patient or potential patient and the health service practitioner, which enables the health service practitioner to provide a remote diagnosis.

FIGS. 102 through 104 portray the use of a data devices, which are connected to the personal computer, to enable a patient or potential patient to send medical data to the health service practitioner through the personal computer and then over the Internet.

FIGS. 105 and 106 provide views of the health service practitioner offering a preliminary diagnosis.

FIG. 107 is a flow chart which exhibits one method of the present invention.

### A DETAILED DESCRIPTION OF PREFERRED & ALTERNATIVE EMBODIMENTS

I. The Remote Diagnostic and Treatment System

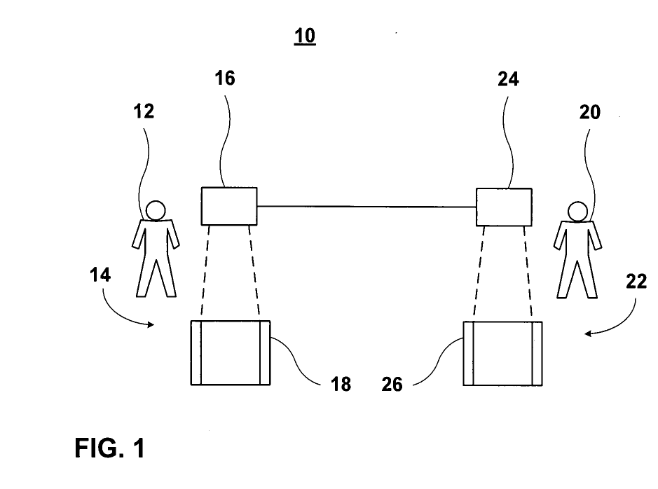

FIG. 1 depicts a Remote Diagnostic & Treatment System 10. In the embodiment shown in FIG. 1, a first person 12 in location 14 has a device or terminal 16, including embedded software 18. The device 16 is used to collect information that is then conveyed to a second person 20 in a second location 22. The second person 20 uses another device or terminal 24 that includes software 26. The second person 20 may use his or her device 24 and software 26 to provide information and assistance to the first person 12. Additionally, the software 26 in device or terminal 24 may be used to control some or all of the features of the device or terminal 16 via its embedded software 18.

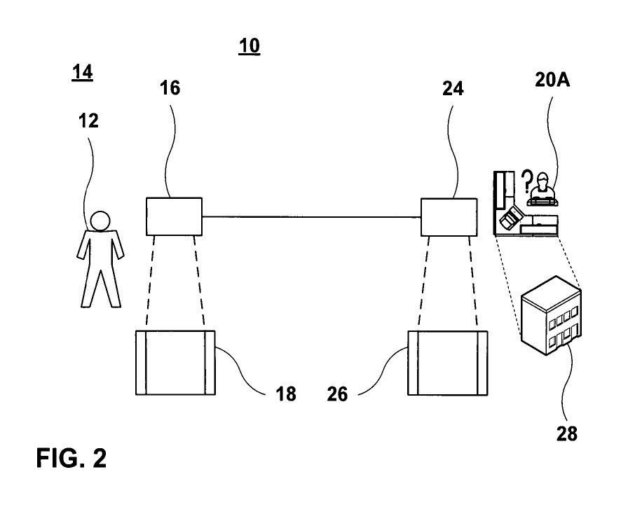

In one specific embodiment of the invention shown in FIG. 2, the first person or user 12 employs his or her first device 16 to contact a call center 28. When used in this Specification and in the Claims that follow, the term “call center” 28 encompasses any facility, establishment or provision for receiving a call, request, message or signal from the first user 12. The call center 28 may comprise a building, facility, place or site staffed by a plurality of operators, technicians, advisors or other personnel. The call center 28 may include any number of live operators 20A, including a single person 20B working in an office, at home or in any other location. In an alternative embodiment, the call center 28 may function without any live human assistance, and may rely on software running on a server, voice recognition equipment, recordings, and/or other automated systems.

Similarly, the term “second person 20” or any other specific individual denotes any combination of persons or automated systems at the other end of the call from the point of view of the first person 12, and may essentially be functionally equivalent to the term “call center” 28.

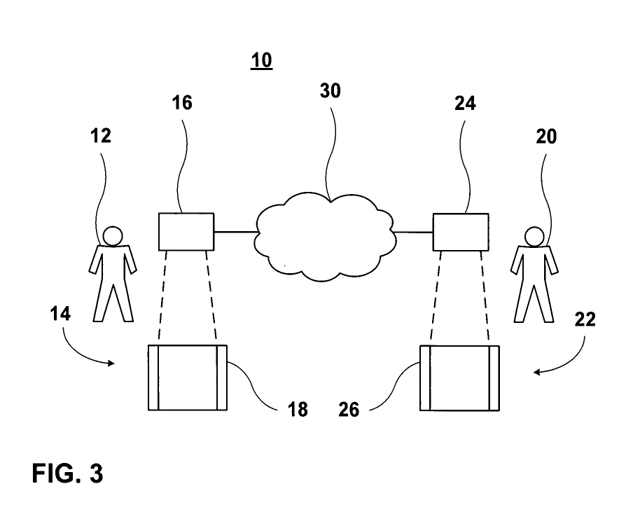

In general, the present invention encompasses any plurality of devices or terminals 16, 24 that are used in combination using a wired or wireless connection over a network 30 as shown in FIG. 3. The network 30 may comprise any combination of wired or wireless connections, including a direct device-to-device link. The first terminal or device 16 is employed to collect, measure, record or otherwise process, store or receive data or information which is then conveyed to another terminal or device 24. The first device 16 has software 18 that enables a user 12 to collect data with the first device 16. The second device 24 has software 26 which enables some form of analysis, examination or response from the user 20 of the second terminal or device 24 back to the user 12 of the first device 16.

The present invention also encompasses any plurality of devices 16, 24 that are used cooperatively to gather information in one place 14, and then use, store, assay, process the data or formulate a response to the data in another place 22. The two locations 14 and 22 may generally be separated by any distance. In addition, the invention provides for the remote direction, monitoring or guidance of the first device 16 by the user 20 of the second device 24. In an alternative embodiment, the user 20 of the second device 24 controls, partially or fully, the operation of the first device 16. In another embodiment, an automated system may control the operation of the first device 16.

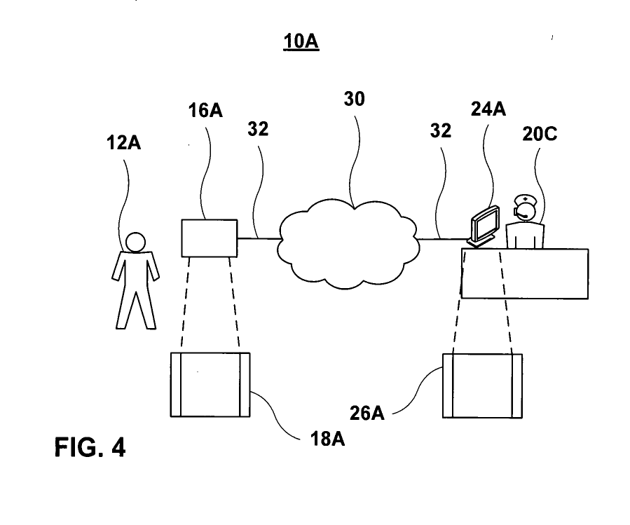

In one particular embodiment of the Remote Diagnostic & Treatment System 10A, which is shown in FIG. 4, the Virtual Visit System™ includes a diagnostic means 16A for collecting data which has diagnostic means software 18A embedded in the diagnostic means 16A that enables remote function control of the diagnostic means 16A. The specific embodiment of the remote diagnostic means 16A collects information about a patient or potential patient 12A. The diagnostic means 16A conveys data over a connection 32 to a network 30 to a diagnostic display and control means 24A which runs one or more software application(s) 26A. In this embodiment, the diagnostic display and control means 24A and software 26A is used by a health service practitioner 20C. In this embodiment, the term “diagnostic” refers to the process of determining or identifying an illness, disease, injury or sickness or other physical or mental condition as a precursor to furnishing an opinion, advice or suggested course of treatment.

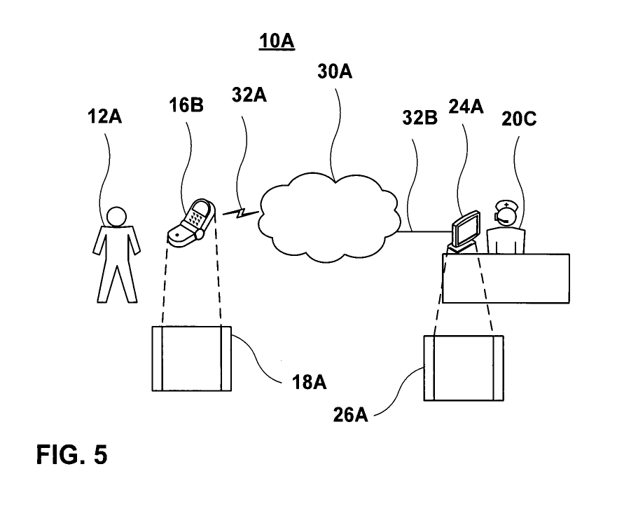

In a specific embodiment of the Remote Diagnostic & Treatment System 10A shown in FIG. 5, the diagnostic means 16A is a cellular or Personal Communications Service (PCS) wireless phone 16B with a wireless network connection 32A to a cellular or PCS network 30A. The cellular or PCS network 30A connects 32B to a health service practitioner's 20C device or terminal 24 via the Public Switched Telephone Network (PSTN) 30B. The diagnostic, display and control means 24A is a computer with a Liquid Crystal Display (LCD) display with one or more software application(s) 26A used by the health service practitioner 20C to remotely control the diagnostic means 16A.

An example of an embodiment of the Remote Diagnostic & Treatment System 10 provides methods and apparatus for undertaking triage, that is, remote diagnosis, and health service monitoring. In this embodiment, the first person is a patient or potential patient 12A having a remote diagnostic device 16A with embedded software 18 that conveys information about the patient or potential patient 12A. In this embodiment, the second person is a health service practitioner 20C.

A second example of an embodiment involves a fire. A fireman on the scene of a fire 14 may use the terminal 16 and its software 18 to collect information about a substance that may have been used to start a fire. Information collected by fireman's terminal 16 may be conveyed to another terminal 24 running different software 26 where the information is analyzed.

A third example involves a crime scene. A police officer at the scene of a crime 14 may collect data concerning evidence of a crime. The policeman may collect this data using his device or terminal 16 and its software 18, and then convey that data to a police headquarters or crime lab 22, where a technician 20 examines the data using his or her own device or terminal 24 which runs software 26.

A fourth example pertains to a construction site. A soil engineer who has obtained a soil sample may use his terminal or device 16 running software 18 to collect data relating to the soil sample, and then conveys the soil sample data to a remote location 22 for analysis by a laboratory technician 20 using his or her own device or terminal 24 and software 26.

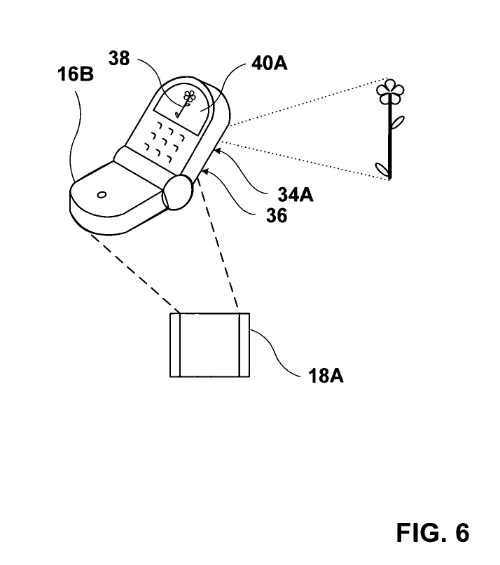

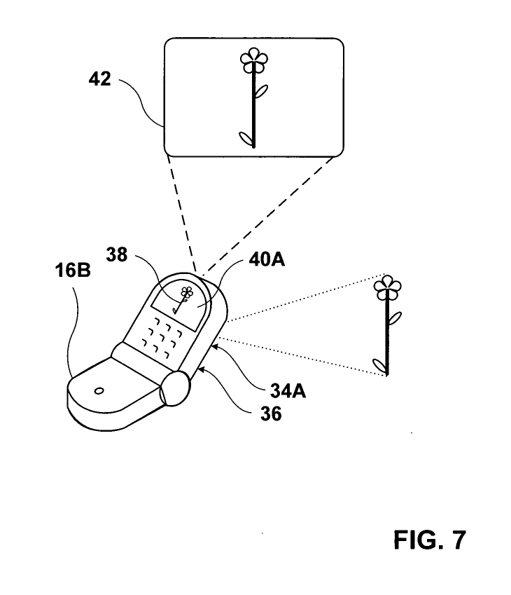

In the first preferred embodiment of the diagnostic means 16A the cellular or PCS wireless phone 16B includes a camera 34A and speaker phone 36A and has embedded software 18A that enables remote function control of the wireless phone 16B, including the camera 34A and speaker phone 36. See FIG. 6. The camera image 38 is displayed on the screen 40A of the cellular or PCS wireless phone 16B. The camera image 38 is a continuous or semi-continuous viewing of what the camera 34A “sees” versus a picture 42 that is a “frozen” instant in time representative of what the camera 34A “sees.” See FIG. 7.

Cellular or PCS wireless phones 16B to which embedded software 18A can be added are available from Audiovox®, Ericcson®, Hewlett Packard®, Kyocera®, LG®, Motorola®, Nokia®, Palm®, Phillips®, Samsung®, Sanyo®, Sony Ericsson®, UT Starcom® and others.

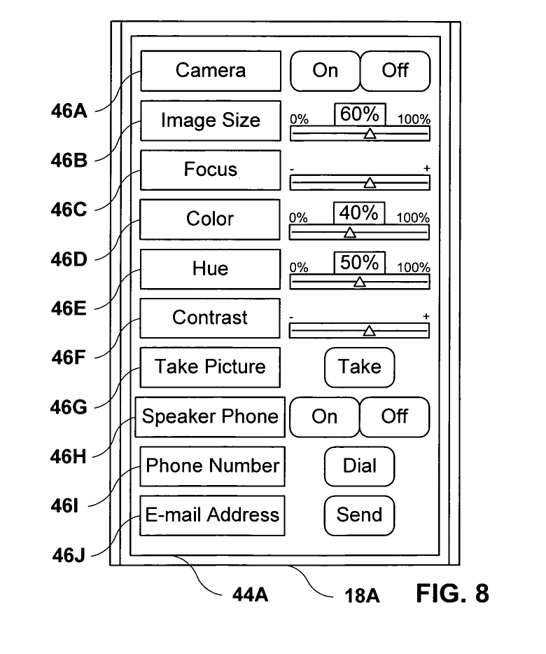

A first preferred embodiment of a functional block diagram 44A of the embedded software 18A that enables remote function control of the cellular or PCS wireless phone 16B is shown in FIG. 8. The functions 46 of a cellular or PCS wireless phone 16B to be controlled include, but are not limited to:
Turn the camera 34A on or off 46A;
“Zoom” the camera image 38 size 46B;
Adjust the camera 34A focus 46C;
Adjust the camera 34A color 46D;
Adjust the camera 34A hue 46E;
Adjust the camera 34A contrast 46F;
Take 46G a picture 42;
Turn 46H the speaker phone 36 on or off;
Enter 46I a telephone number 48 to which to send the picture 42; and
Enter 46J an electronic mail (e-mail) address 50 to which to send the picture 42.

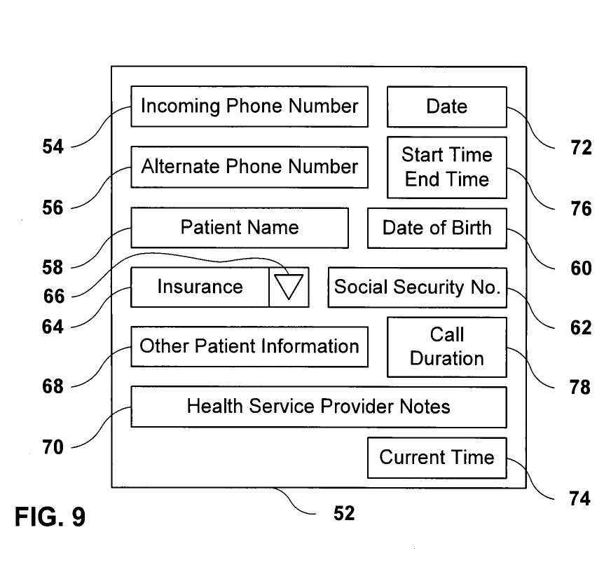

When a health service practitioner 20C receives a call from a patient or potential patient 12A the first element of the diagnostic, display and control software application 26A deployed on a diagnostic, display and control means 26A is the screen 52 shown in FIG. 9. A preferred embodiment of the software application 26A captures the incoming phone number 54 using caller identification (CID). If for some reason CID is not available, the health service practitioner 20C asks the patient or potential patient 12A for his or her telephone number 54 and enters the incoming phone number 54 into the screen 52 so that if there is an interruption in communications, the health service practitioner 20C can call the patient or potential patient 12A back. The second thing the health service practitioner 20C ascertains is an alternate phone number 56 to reach the patient or potential patient 12A in the event communications cannot be reestablished via the incoming phone number 54.

Once communications are assured, the health service practitioner 20C gets the patient's or potential patient's 12A name 58, date of birth 60, Social Security Number (SSN) 62, and insurance carrier 64. Insurance carriers 64 utilizing a particular Remote Diagnostic & Treatment System 10A may be accessible to the health service practitioner 20C via a pull down menu 66.

In a preferred embodiment of a Remote Diagnostic & Treatment System 10A, a patient's 12A medical records are available on-line and are displayed 68 once the patient's name 58, date of birth 60 and SSN 62 are entered.

The screen 52 also contains an area 70 for the health service practitioner 20C to enter notes about his or her interaction with the patient or potential patient 12A.

The software application 26A automatically notes the date of the phone call 72, the current time 74, the start time and end time 76 of the phone call as well as the call duration 78.

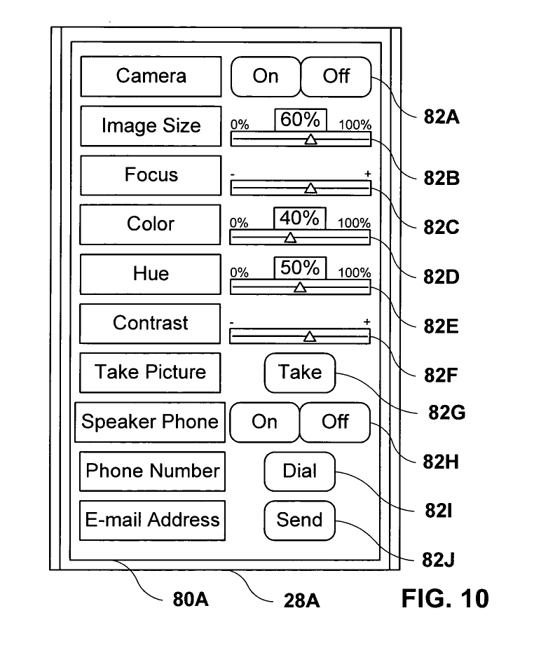

A first preferred embodiment of a block diagram 80A for the diagnostic, display and control software application 26A deployed on a diagnostic, display and control means 24A used by a health service practitioner 20C to control a cellular or PCS wireless phone 16B is shown in FIG. 10. The remote functional control means 82 for a cellular or PCS wireless phone 16B include, but are not limited to:
An “off” button and an “on” button 82A for controlling 46A the camera 34A;
A slider 82B that “zooms” the camera image 38 size 46B from zero percent (0%) to one hundred percent (100%);
A slider 82C that adjusts the camera focus 46C from minus (−) to plus (+);
A slider 82D that adjusts the color 46D from zero percent (0%) to one hundred percent (100%);
A slider 82E that adjusts the hue 46E from zero percent (0%) to one hundred percent (100%);
A slider 82F that adjusts the contrast 46F from minus (−) to plus (+);
A button 82G for taking 46G a picture 36;
An “off” button and an “on” button 82H for controlling 46H the speaker phone 36;
A button 82I for dialing 461 the phone number 48 to which the picture 42 is to be sent; and
A button 82J for sending 46J the picture 42 to an e-mail address 50.

The diagnostic, display and control software application 26A deployed on a diagnostic, display and control means 24A used by a health service practitioner 20C has to have the ability to display the camera image 38 and the picture 42.

Camera-equipped 34A cellular and PCS phones 16A have embedded software that allows a user to take a picture 42 and send it to a second user of a camera-equipped 34A cellular or PCS phone 16B. The software application 26A must have the same capability so that the health service practitioner 20C can view the picture 42. Today's camera-equipped 34A cellular or PCS phone 16B are limited to sending pictures 42; future phones are expected to allow short video clips to be sent.

If the picture 42 is to be e-mailed, then the software application 26A must have access to the Internet and an e-mail application that allows the health service practitioner 20C to view the picture 42.

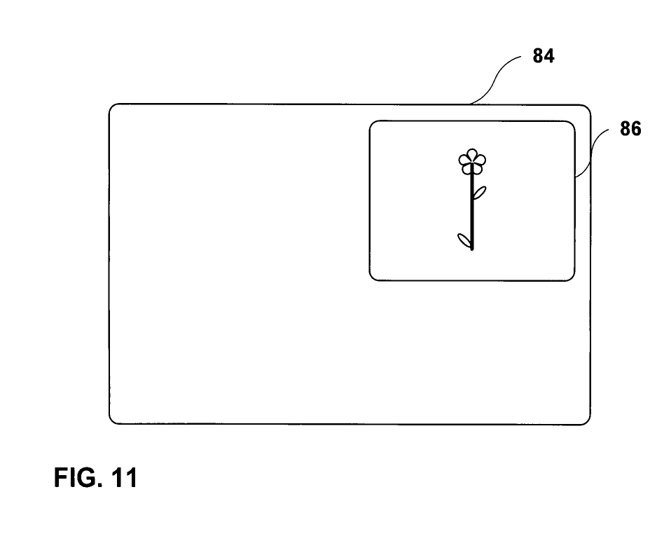

FIG. 11 shows a screen 84 displayed on a diagnostic, display and control means 24A used by a health service practitioner 20C. A transmitted picture 42 appears in the window 86.

In this Specification and in the Claims that follow, the term “partially control” refers to a joint or cooperative sharing of the control of the features of the terminal or cellular phone 16A by both the user and a another person, such as an operator 20A at a call center 28. The operator 20A may control some or all of the features of the cellular phone 16A.

II. Data Devices

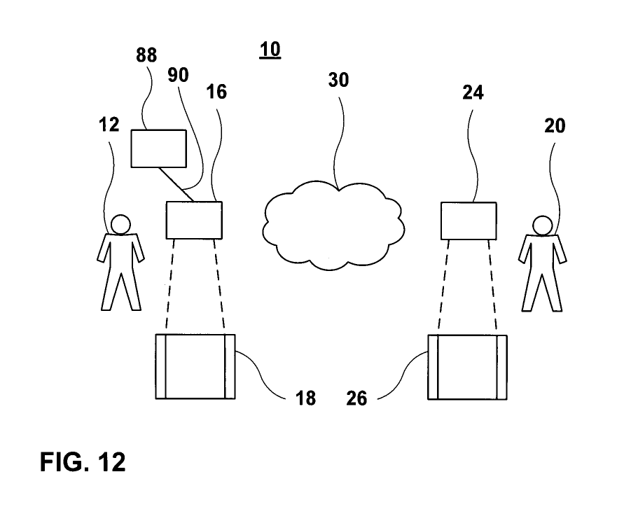

Many different data devices 88 can enhance the effectiveness of the Remote Diagnostic & Treatment System 10. These data devices 88 are connected 90 to the device or terminal 16 as shown in FIG. 12. There are numbers of technologies that may be used for the connection 90 between the data devices 88 and the device or terminal 16, both wired and wireless.

Numbers of cellular and PCS wireless phones 16A include Bluetooth® 90A, a low-power radio communications to wirelessly link phones, computers and other network devices over short distances. Wireless signals transmitted with Bluetooth cover short distances, typically up to thirty feet (30 ft) or ten meters (10 m).

“WiFi” 90B is an abbreviation for “wireless fidelity,” a wireless local area network (WLAN) that conforms to the Institute of Electrical and Electronics Engineers (IEEE) specification 802.11. Some cellular and PCS wireless phones 16A also include WiFi capabilities.

Ultra Wide Band (UWB) 90C is a wireless technology that uses less power and provides higher data speed than WiFi or Bluetooth and has the ability to carry signals through doors and other obstacles that tend to reflect signals at more limited bandwidths and a higher power. UWB chip sets and their concomitant support software are just now becoming available. Cellular and PCS wireless phones 16A with UWB capabilities are just being commercially announced.

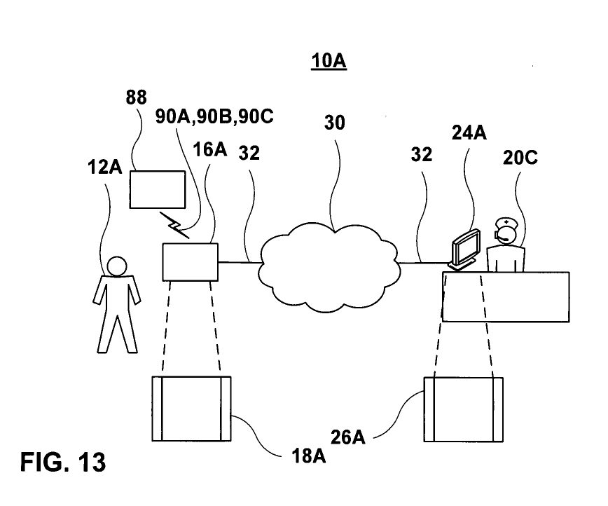

Wireless connections 90A, 90B, 90C between the data devices 88 and the device or terminal 16 are shown in FIG. 13.

A first embodiment of a wired connection between an data device 88 and a device or terminal 16 is Universal Serial Bus (USB) 90D, an external bus standard that supports data transfer rates of 12 Mbps for up to one hundred twenty-seven peripheral devices.

A second embodiment of a wired connection between an data device 88 and a device or terminal 16 is FireWire 84E, IEEE 1394, High Performance Serial Bus. FireWire provides a single plug-and-socket connection on which up to sixty-three devices can be attached with data transfer speeds up to four hundred megabits per second.

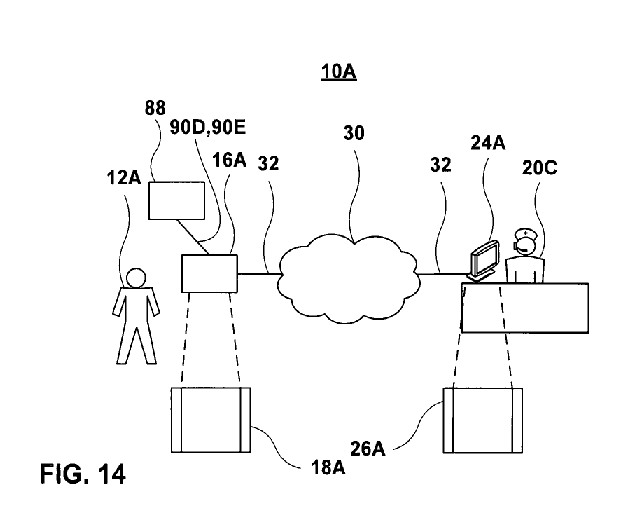

Wired connections 90D, 90E between data devices 88 and the device or terminal 16 are shown in FIG. 14.

For each of the data devices 88 to connect 90 to a remote device or terminal 16, there must be a connection interface device 92 that accepts the data from the data device 88, and configures it for the connection 90 to the device or terminal 16.

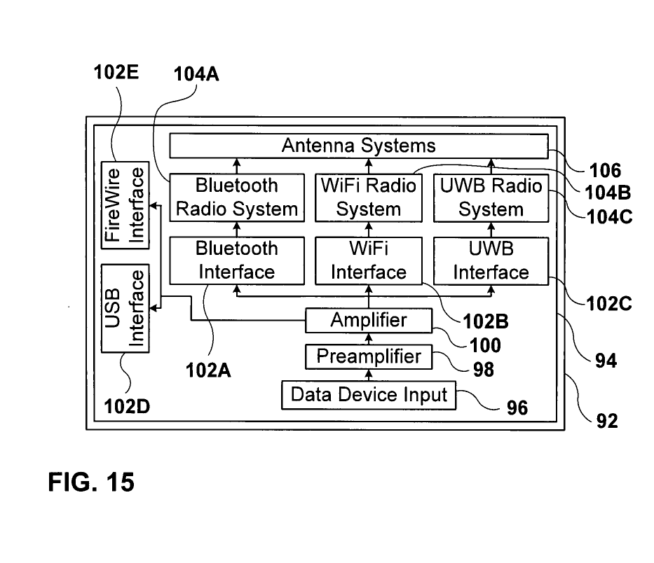

FIG. 15 shows a functional block diagram 94 for the connection interface device 92. Device data input 96 is fed to a preamplifier 98 and then an amplifier 100. Thereafter the amplified data input 96 is fed into the appropriate interface 102 for the connection 90 to be used; the Bluetooth interface 102A for Bluetooth 90A, the WiFi interface 102B for WiFi 90B, the UWB interface 102C for UWB 90C, the USB interface 102D for USB 90D, and the FireWire interface 102E for FireWire 90E. The output of the USB interface 102D is the USB connection 90D; the output of the FireWire interface 102E is the FireWire connection 90E. The output of the Bluetooth interface 102A is fed into the Bluetooth radio system 104A and then to the antenna system 106. Similarly, the output of the WiFi interface 102B is fed to into the WiFi radio system 104B and then to the antenna system 106. The output of the UWB interface 102C is fed into the UWB radio system 104C and then to the antenna system 106.

In one embodiment, the connection interface device 92 is built into the data devices 88. In one embodiment, one or more connection 90 technologies is built into each data device 88.

Thermometer

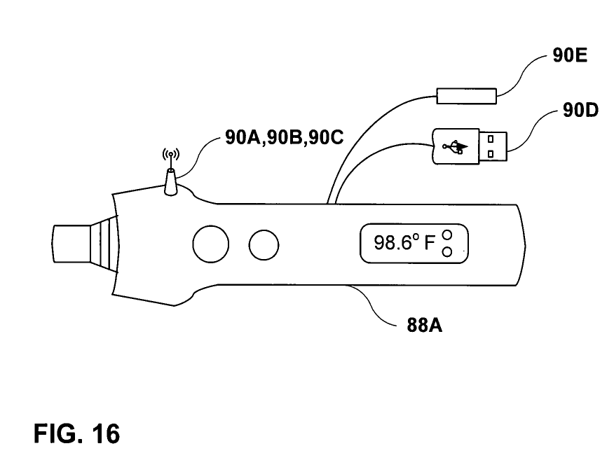

The first data device 88 is a thermometer 88A. Many different contact-less digital thermometers 88A are commercially available from Bebesounds®, Braun®, EJK®, Lumiscope®, Mabis Healthcare®, Samsumg® and others. A preferred embodiment of a thermometer 88A to be deployed in the Remote Diagnostic & Treatment System 10A is shown in FIG. 16, and includes a connection 90 to a device or terminal 16. The embodiment shown in FIG. 16 includes one or more wireless connections, Bluetooth 90A, WiFi 90B and UWB 90C, and one or more wired connections, USB 90D and FireWire 90E.

For a cellular or PCS wireless phone 16B to receive the temperature reading from the thermometer 88A, it must have embedded software 18A that recognizes that a digital temperature reading is being sent to the wireless phone 16B. One embodiment of the embedded software 18A allows the temperature to be displayed on the screen 40A of a wireless phone 16B. Having received the temperature reading from the thermometer 88A, there must be additional software 18A to forward the temperature reading to the diagnostic, display and control software application 26A deployed on the diagnostic, display and control means 24A used by a health service practitioner 20C.

Similarly, the diagnostic, display and control software application 26A deployed on a diagnostic, display and control means 24A used by a health service practitioner 20C has to have the ability to display the temperature reading received from the cellular or PCS wireless phone 16B. The health service practitioner 20C must have the ability to take or retake the temperature reading, as well as to determine whether to display the temperature reading on the cellular or PCS wireless phone 16B.

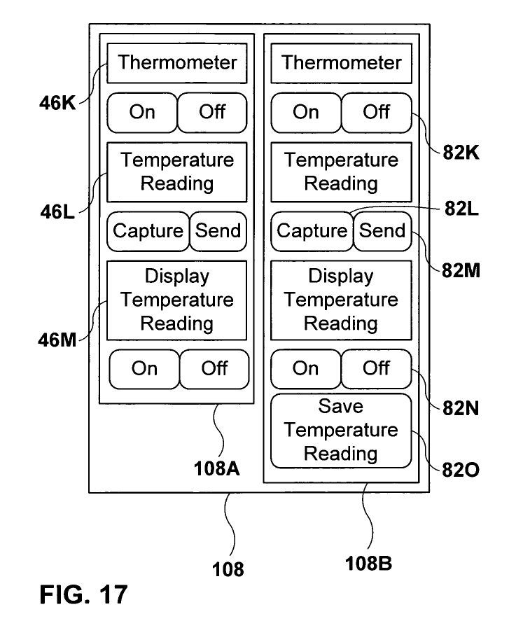

FIG. 17 shows a first embodiment of a functional block diagram 108 of embedded software 18A that enables remote functional control of the data devices 88 for the diagnostic means 16A as well as a software application 26A that enables remote functional control of the data devices 88 connected to the diagnostic means 16A. The functions shown in 108A are deployed as a component of 18A, the software embedded in a cellular and PCS wireless phone 16B; those in 108B as a component of the diagnostic, display and control software application 26A.

The functions 46 embedded in a cellular or PCS wireless phone 16B for controlling the data device 88 the thermometer 88A include, but are not limited to:
Turn the thermometer 88A on or off 46K;
Capture and send the temperature reading 46L; and
Turn the temperature reading display 46M of the cellular and PCS wireless phone 16B on or off.

The functional control means 82 in the diagnostic, display and control software application 26A for remotely controlling the thermometer 88A via a cellular or PCS wireless phone 16B include, but are not limited to:
An “off” button and an “on” button 82K for controlling 46K the thermometer 88A;
A button 82L for capturing or recapturing the temperature reading 46L;
A button 82M for sending the temperature reading 46L to a health service practitioner 14; and
An “off” button and an “on” button 82N for controlling the display 46M of the temperature reading 46L on a cellular and PCS wireless phone 16B.

An additional functional control means 82O allows the health service practitioner 20C to save the temperature reading 46L to the patient's or potential patient's 12A electronic medical file. The diagnostic, display and control software application 26A automatically tags the temperature reading 46L with the date 72 and current time 74.

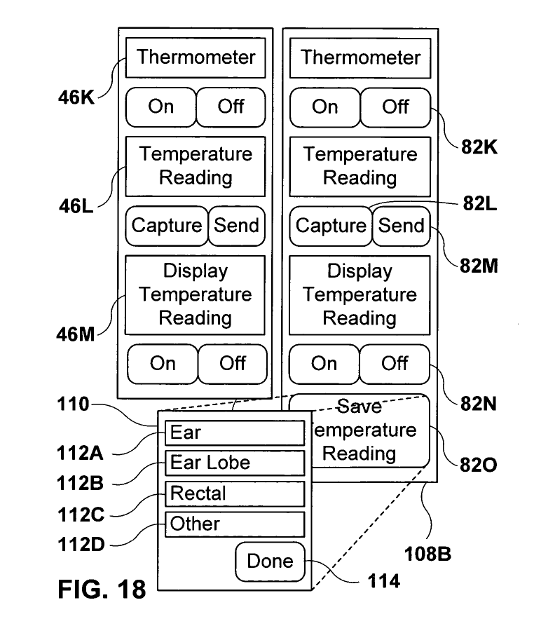

When button 82O is pushed, pop-up window 110 appears on the diagnostic, display and control means 24A allowing the health service practitioner 20C to note where on the patient's or potential patient's 12A body the temperature reading 46L was taken 112, as shown in FIG. 18. If the location 112 is not listed in pop-up window 110, the health service practitioner 20C enters the location in the “other” box 112D. Selecting one of 112A through 112C automatically closes pop-up window 110. If information is entered into 112D, the health service practitioner 20C clicks the “done” button 114 to close the pop-up window 110.

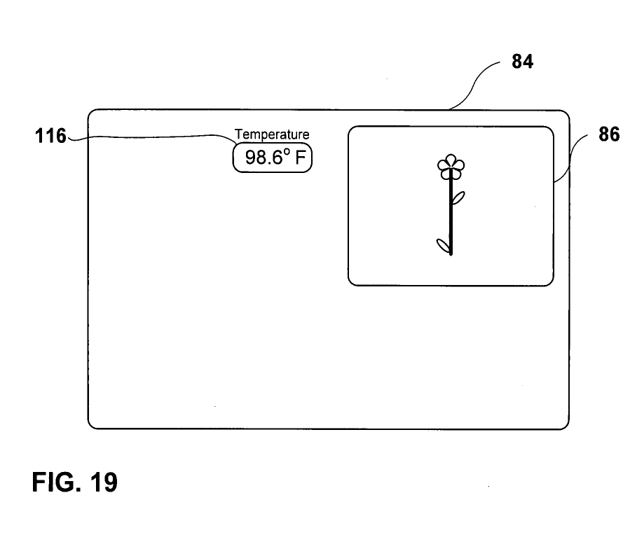

The temperature reading 46L appears in window 116 on the health service practitioner's 20C diagnostic, display and control means 24A as shown in FIG. 19.

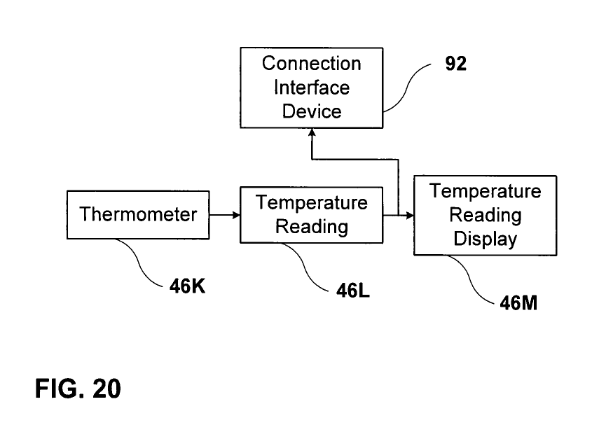

FIG. 20 shows the temperature reading 46L fed to the connection interface device 92 for connection 90 to the device or terminal 16.

Stethoscope or High Fidelity Microphone

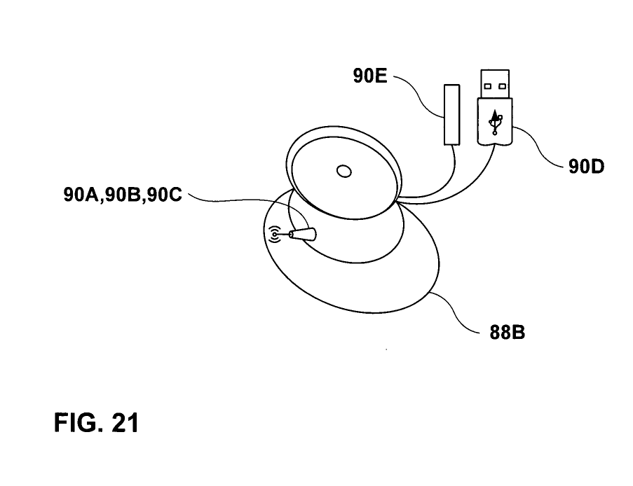

A second data device 88 is an acoustic sensor, such as a stethoscope or high fidelity microphone 88B. A stethoscope or high fidelity microphone 88B is used to listen to the heart and lungs of a patient or potential patient 12A as well as to capture pulse rate. Numbers of stethoscopes 88B are commercially available from AllHeart®, American Diagnostic Corporation (ADC)®, Doctors Research Group (DRG)®, Heine®, Prestige Medical®, 3M Littmann®, UltraScopes®, W. A. Baum®, WelchAllyn® and others. High fidelity microphones are commercially available from AKG®, Audio-Technica®, Beyerdynamics®, Sennheiser®, Shure®, Sony® and others. A preferred embodiment of a stethoscope or high fidelity microphone 88B to be deployed in the Remote Diagnostic & Treatment System 10A is shown in FIG. 21, and includes a connection 90 to a device or terminal 16. The embodiment shown in FIG. 21 includes one or more wireless connections, Bluetooth 90A, WiFi 90B and UWB 90C, and one or more wired connections, USB 90D and FireWire 90E.

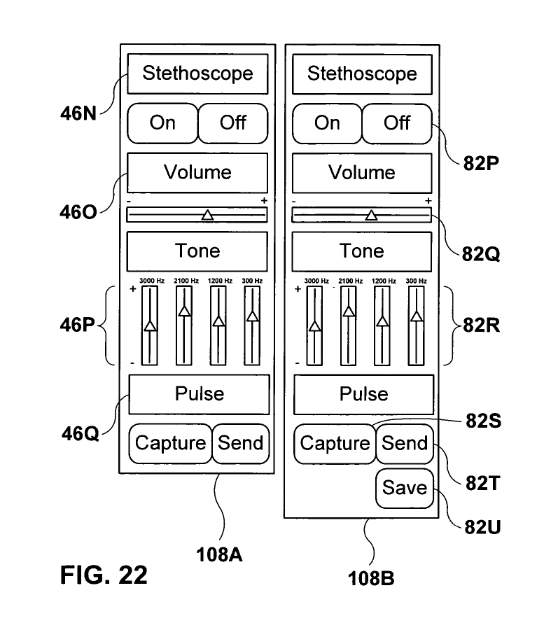

FIG. 22 shows a second embodiment of a functional block diagram 108 of embedded software 18A that enables remote functional control of the data devices 88 for the diagnostic means 16A as well as a software application 26A that enables remote functional control of the data devices 88 connected to the diagnostic means 16A. The functions shown in 108A are deployed as a component of 18A, the software embedded in a cellular and PCS wireless phone 16B; those in 108B as a component of the diagnostic, display and control software application 26A.

The pulse rate is automatically captured by either the embedded software that enables remote function control 18A or the diagnostic, display and control software application 26A, basically by listening to the heart beats and measuring them against the time 74.

The functions 46 embedded in a cellular or PCS wireless phone 16B for controlling the data device 88 the stethoscope or high fidelity microphone 88B include, but are not limited to:
Turn the stethoscope or high fidelity microphone 88B on or off 46N;
Adjust the volume 46O of the stethoscope or high fidelity microphone 88B;
Adjust the tone 46P of the stethoscope or high fidelity microphone 88B using an equalizer; and
Capture and send the pulse 46Q.

The functional control means 82 in the diagnostic, display and control software application 26A for remotely controlling the stethoscope or high fidelity microphone 88B via a cellular or PCS wireless phone 16B include, but are not limited to:
An “off” button and an “on” button 82P for controlling 46N the stethoscope or high fidelity microphone 88B;
A slider 82Q that adjusts the volume 46O of the stethoscope or high fidelity microphone 88B from minus (−) to plus (+);
Multiple sliders 82R that adjust the tone 46P of the stethoscope or high fidelity microphone 88B from minus (−) to plus (+);
A button 82S for capturing or recapturing the pulse reading 46Q; and
A button 82T for sending the pulse reading 46Q to a health service practitioner 14.

An additional functional control means 82U allows the health service practitioner 20C to save the pulse reading 46Q to the patient's or potential patient's 12A electronic medical file. The software application 26A automatically tags the pulse reading 46Q with the date 72 and current time 74.

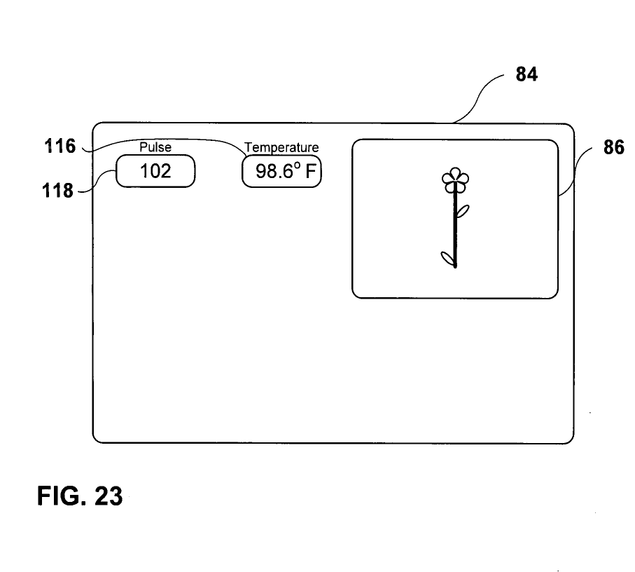

The pulse reading 46Q appears in window 118 on the health service practitioner's 20C diagnostic, display and control means 24A as shown in FIG. 23.

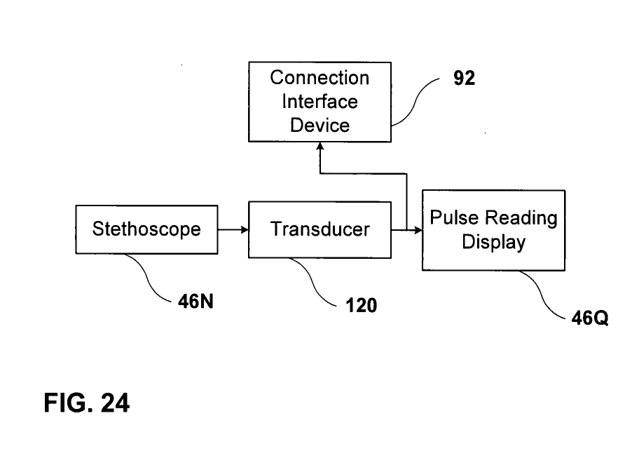

A stethoscope is basically a cavity resonator that amplifies sound; there are no electronic components. Microphone components, a transducer 120, must be added to a stethoscope to convert sound waves to electrical signals. The stethoscope or high fidelity microphone 88B must have an interface that captures the sound signals and makes those signals available to the connection 90 to the device or terminal 16. This is accomplished via the connection interface device 92 shown in FIG. 24.

Weight Scale

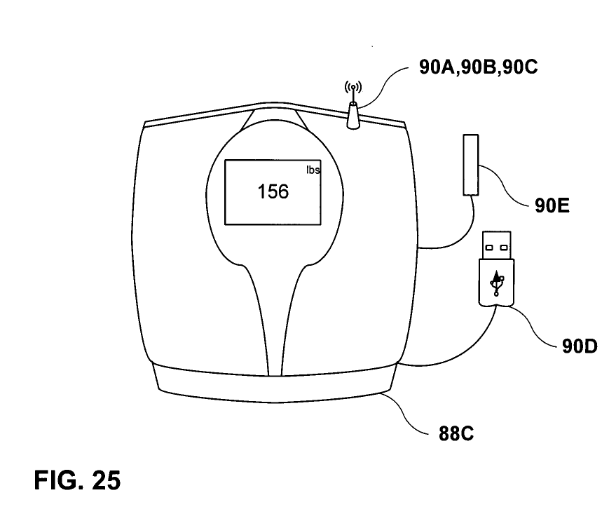

The third data device 88 is a weight measurement device, such as a scale 88C. Numbers of weight scales 88C are commercially available from Braun®, Health-O-Meter®, Homedics®, LifeSource®, MedWeigh®, Rowenta®, Soehnle®, Tanita® and others. A preferred embodiment of a weight scale 88C to be deployed in the Remote Diagnostic & Treatment System 10A is shown in FIG. 25, and includes a connection 90 to a device or terminal 16. The embodiment shown in FIG. 25 includes one or more wireless connections, Bluetooth 90A, WiFi 90B and UWB 90C, and one or more wired connections, USB 90D and FireWire 90E.

For a cellular or PCS wireless phone 16B to receive the weight reading from the scale 88C, it must have embedded software 18A that recognizes that a digital weight reading is being sent to the wireless phone 16B. One embodiment of the embedded software 18A allows the weight to be displayed on the screen 40A of the wireless phone 16B. Having received the weight reading from the scale 88C, there must be additional software 18A to forward the weight reading to the diagnostic, display and control means 24A used by a health service practitioner 20C.

Similarly, the diagnostic, display and control software application 26A deployed on a diagnostic, display and control means 24A used by a health service practitioner 20C has to have the ability to display the weight reading received from the cellular or PCS wireless phone 16B. The health service practitioner 20C must have the ability to take or retake the weight reading, as well as to determine whether to display the weight reading on the cellular or PCS wireless phone 16B.

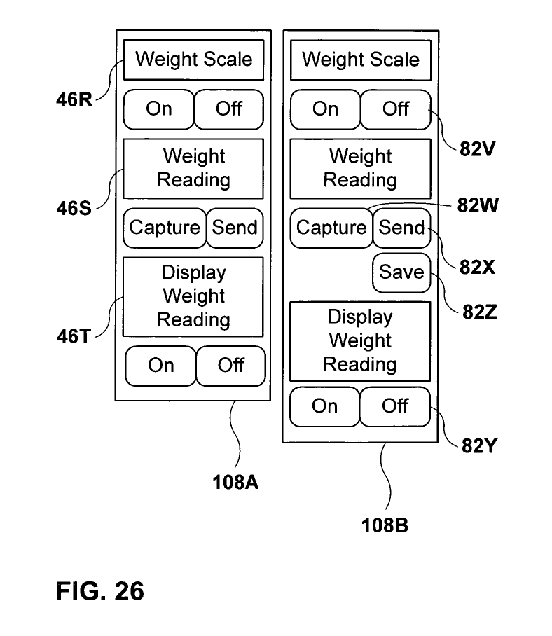

FIG. 26 shows a third embodiment of a functional block diagram 108 of embedded software 18A that enables remote functional control of the data devices 88 for the diagnostic means 16A as well as a software application 26A that enables remote functional control of the data devices 88 connected to the diagnostic means 16AA. The functions shown in 108A are deployed as a component of 18A, the software embedded in a cellular and PCS wireless phone 16B; those in 108B as a component of the diagnostic, display and control software application 26A.

The functions 46 embedded in a cellular or PCS wireless phone 16B for controlling the data device 88 the weight scale 88C include, but are not limited to:
Turn the scale 88C on or off 46R;
Capture and send the weight reading 46S; and
Turn the weight reading display 46T on or off.

The functional control means 82 in the diagnostic, display and control software application 26A for remotely controlling the scale 88C via a cellular or PCS wireless phone 16B include, but are not limited to:
An “off” button and an “on” button 82V for controlling 46R the scale 88C;
A button 82W for capturing or recapturing the weight reading 46S;
A button 82X for sending the weight reading 46S to a health service practitioner 14; and
An “off” button and an “on” button 82Y for controlling the display 46T of the weight reading 46S.

An additional functional control means 82Z allows the health service practitioner 20C to save the weight reading 46S to the patient's or potential patient's 12A electronic medical file. The software application 26A automatically tags the weight reading 46S with the date 72 and current time 74.

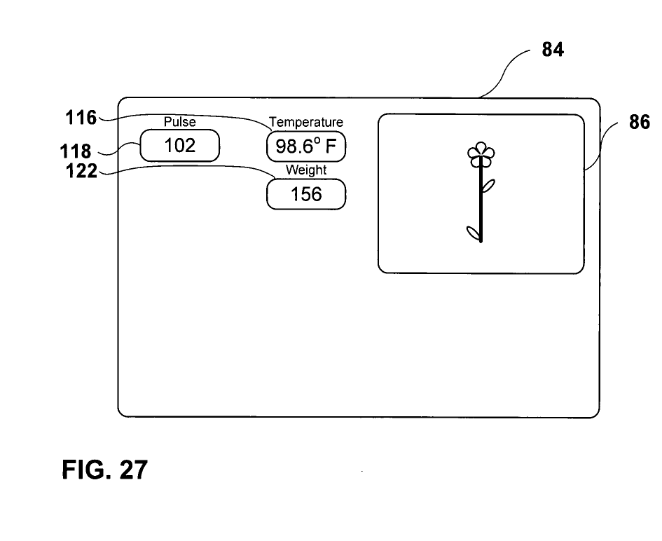

The weight reading 46S appears in window 122 on the health service practitioner's 20C diagnostic, display and control means 24A as shown in FIG. 27.

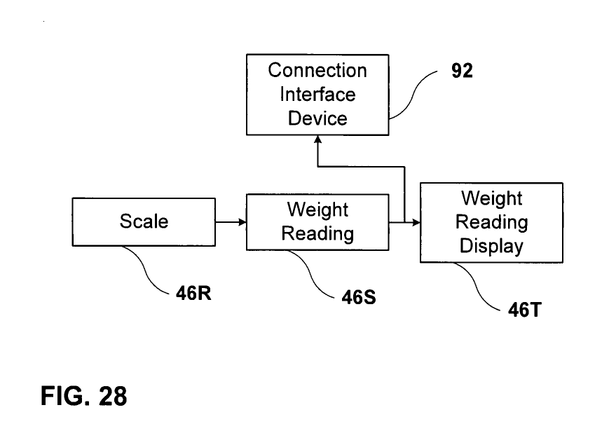

FIG. 28 shows the weight reading 46S fed to the connection interface device 92 for connection 90 to the device or terminal 16.

Blood Pressure Cuff

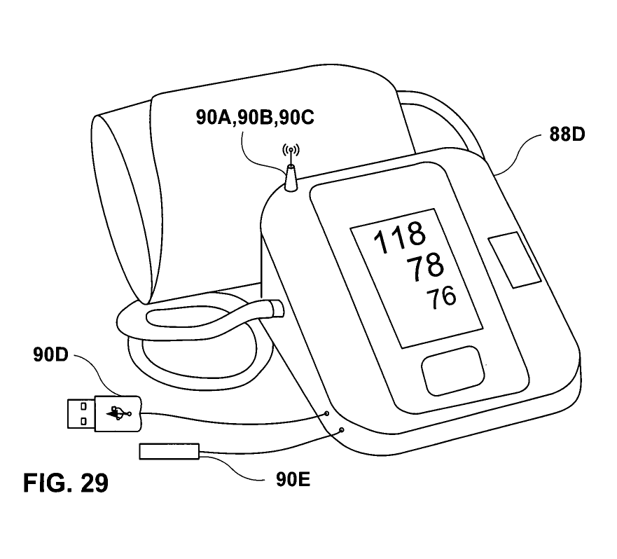

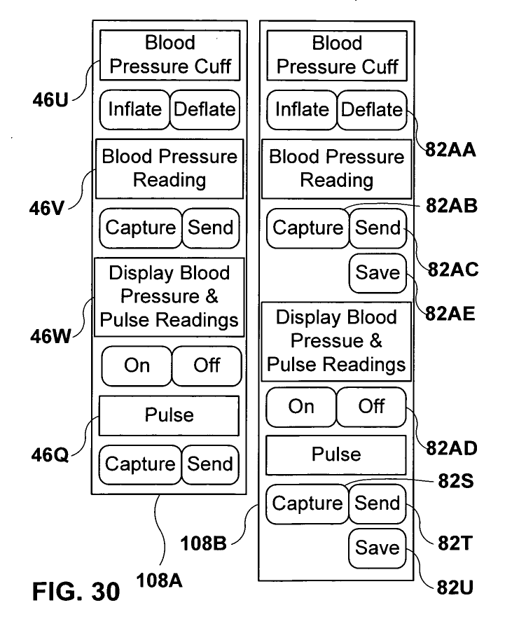

The fourth data device 88 is a blood pressure measurement device, such as a cuff 88D. Numbers of blood pressure cuffs 88D are commercially available from Health-O-Meter®, Hitachi®, Lumiscope®, Mabis®, Microlife®, Omron®, Oregon Scientific®, Panasonic®, Samsung® and others. A preferred embodiment of a blood pressure cuff 88D to be deployed in the Remote Diagnostic & Treatment System 10A is shown in FIG. 29, and includes a connection 90 to a device or terminal 16. The embodiment shown in FIG. 29 includes one or more wireless connections, Bluetooth 90A, WiFi 90B and UWB 90C, and one or more wired connections, USB 90D and FireWire 90E.

For a cellular or PCS wireless phone 16B to receive the blood pressure and pulse readings from the blood pressure cuff 88D, it must have embedded software 18A that recognizes that digital blood pressure and pulse readings are being sent to the wireless phone 16B. One embodiment of the embedded software 18A allows the blood pressure and pulse readings to be displayed on the screen 40A of the wireless phone 16B. Having received the blood pressure and pulse readings from the blood pressure cuff 88D, there must be additional software 18A to forward the blood pressure and pulse readings to the diagnostic, display and control means 24A used by a health service practitioner 20C.

Similarly, the diagnostic, display and control software application 26A deployed on a diagnostic, display and control means 24A used by a health service practitioner 20C has to have the ability to display the blood pressure and pulse readings received from the cellular or PCS wireless phone 16B. The health service practitioner 20C must have the ability to take or retake the blood pressure and pulse readings, as well as to determine whether to display the weight reading on the cellular or PCS wireless phone 16B.

FIG. 30 shows a fourth embodiment of a functional block diagram 108 of embedded software 18A that enables remote functional control of the data devices 88 for the diagnostic means 16A as well as a software application 26A that enables remote functional control of the data devices 88 connected to the diagnostic means 16A. The functions shown in 108A are deployed as a component of 18A, the software embedded in a cellular and PCS wireless phone 16B; those in 108B as a component of the diagnostic, display and control software application 26A.

The functions 46 embedded in a cellular or PCS wireless phone 16B for controlling the data device 88 the blood pressure cuff 88D include, but are not limited to:
Inflate or deflate 46U the blood pressure cuff 88D;
Capture and send the blood pressure reading 46V;
Capture and send the pulse reading 46Q; and
Turn the blood pressure and pulse readings display 46W on or off.

The functional control means 82 in the diagnostic, display and control software application 26A for remotely controlling the blood pressure cuff 88D via a cellular or PCS wireless phone 16B include, but are not limited to:
A button 82AA for inflating and deflating 46U the blood pressure cuff 88D;
A button 82AB for capturing or recapturing the blood pressure reading 46V;
A button 82AC for sending the blood pressure reading 46V to a health service practitioner 14;
A button 82S for capturing or recapturing the pulse reading 46Q; and
A button 82T for sending the pulse reading 46Q to a health service practitioner 14.
An “off” button and an “on” button 82AD for controlling the display 46W of the blood pressure and pulse readings 46W.

An additional functional control means 82AE allows the health service practitioner 20C to save the blood pressure reading 46V to the patient's or potential patient's 12A electronic medical file. The software application 26A automatically tags the blood pressure reading 46V with the date 72 and current time 74.

An additional functional control means 82U allows the health service practitioner 20C to save the pulse reading 46Q to the patient's or potential patient's 12A electronic medical file. The software application 26A automatically tags the pulse reading 46Q with the date 72 and current time 74.

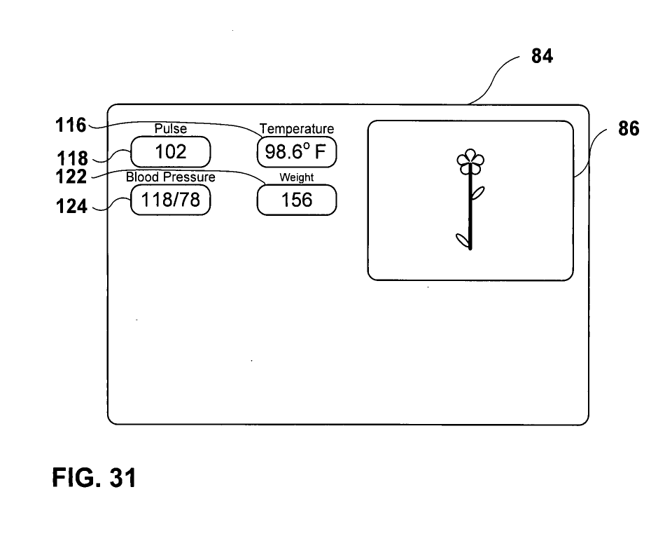

The pulse reading 46Q appears in window 118 on the health service practitioner's 20C diagnostic, display and control means 24A as shown in FIG. 31.

The blood pressure reading 46V appears in window 124 on the health service practitioner's 20C diagnostic, display and control means 24A as shown in FIG. 31.

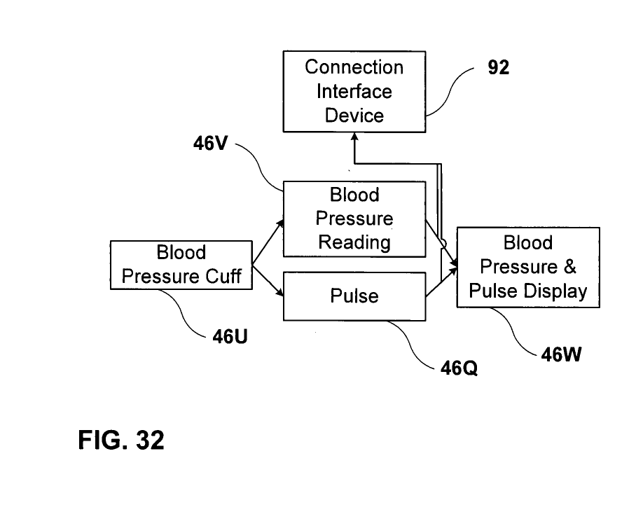

FIG. 32 shows the pulse 46Q and the blood pressure reading 46V fed to the connection interface device 92 for connection 90 to the device or terminal 16.

Oximeter

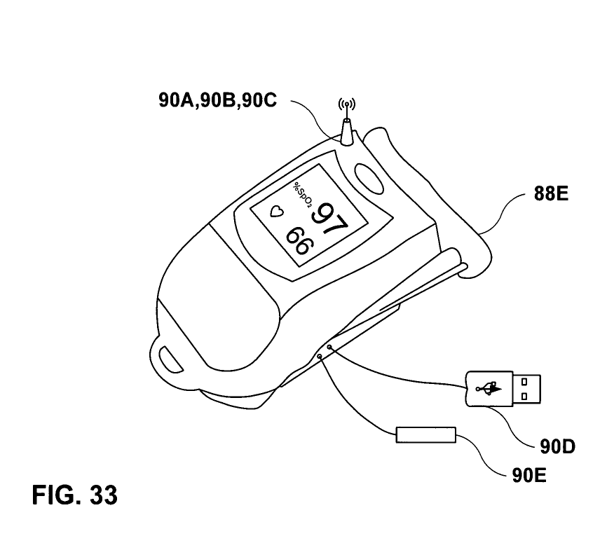

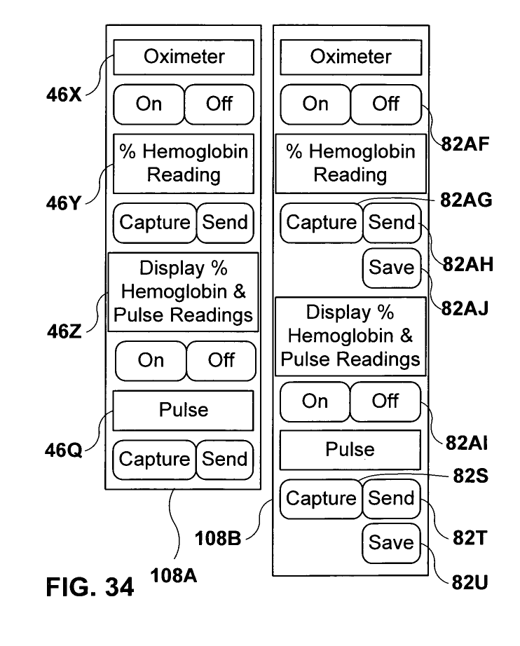

The fifth data device 88 is a device which measures levels of oxygen in the blood, such as an oximeter 88E. Numbers of oximeters 88E are commercially available from BCI®, Criticare®, INVOS®, Nonin Medical®, Smiths Medical PM Inc.®, SPO®, Turner Medical® and others. The Nonin Medical Inc. Avant™ 4600 Digital Pulse Oximetry System sends pulse rate data from a wrist-worn sensor to a monitor via Bluetooth. A preferred embodiment of an oximeter 88E to be deployed in the Remote Diagnostic & Treatment System 10A is shown in FIG. 33, and includes a connection 90 to a device or terminal 16. The embodiment shown in FIG. 33 includes one or more wireless connections, Bluetooth 90A, WiFi 90B and UWB 90C, and one or more wired connections, USB 90D and FireWire 90E.

For a cellular or PCS wireless phone 16B to receive the reading of the percent of hemoglobin that is saturated with oxygen and pulse reading from the oximeter 88E, it must have embedded software 18A that recognizes that the reading of the percent of hemoglobin that is saturated with oxygen and pulse reading are being sent to the wireless phone 16B. One embodiment of the embedded software 18A allows the reading of the percent of hemoglobin that is saturated with oxygen and pulse reading to be displayed on the screen 40A of the wireless phone 16B. Having received the reading of the percent of hemoglobin that is saturated with oxygen and pulse reading from the oximeter 88E, there must be additional software 18A to forward the reading of the percent of hemoglobin that is saturated with oxygen and pulse reading to the diagnostic, display and control means 24A used by a health service practitioner 20C.

Similarly, the diagnostic, display and control software application 26A deployed on a diagnostic, display and control means 24A used by a health service practitioner 20C has to have the ability to display the reading of the percent of hemoglobin that is saturated with oxygen and pulse reading received from the cellular or PCS wireless phone 16B. The health service practitioner 20C must have the ability to take or retake the reading of the percent of hemoglobin that is saturated with oxygen and pulse reading, as well as to determine whether to display the readings on the cellular or PCS wireless phone 16B.

FIG. 34 shows a fifth embodiment of a functional block diagram 108 of embedded software 18A that enables remote functional control of the data devices 88 for the diagnostic means 16A as well as a software application 26A that enables remote functional control of the data devices 88 connected to the diagnostic means 16A. The functions shown in 108A are deployed as a component of 18A, the software embedded in a cellular and PCS wireless phone 16B; those in 108B as a component of the diagnostic, display and control software application 26A.

The functions 46 embedded in a cellular or PCS wireless phone 16B for controlling the data device 88 the oximeter 88E include, but are not limited to:
Turn the oximeter 88E on or off 46X;
Capture and send the reading of the percent of hemoglobin that is saturated with oxygen 46Y;
Capture and send the pulse reading 46Q; and
Turn the reading of the percent of hemoglobin that is saturated with oxygen and pulse reading display 46Z on or off.

The functional control means 82 in the diagnostic, display and control software application 26A for remotely controlling the oximeter 88E via a cellular or PCS wireless phone 16B include, but are not limited to:
An “off” button and an “on” button 82AF for controlling 46X the oximeter 88E;
A button 82AG for capturing or recapturing the reading of the percent of hemoglobin that is saturated with oxygen 46Y;
A button 82AH for sending the reading of the percent of hemoglobin that is saturated with oxygen 46Y to a health service practitioner 14;
A button 82S for capturing or recapturing the pulse reading 46Q; and
A button 82T for sending the pulse reading 46Q to a health service practitioner 14;
An “off” button and an “on” button 82AI for controlling the display 46Z of the reading of the percent of hemoglobin that is saturated with oxygen and pulse reading.

An additional functional control means 82AJ allows the health service practitioner 20C to save the reading of the percent of hemoglobin that is saturated with oxygen 46Y to the patient's or potential patient's 12A electronic medical file. The software application 26A automatically tags the blood pressure reading 46V with the date 72 and current time 74.

An additional functional control means 82U allows the health service practitioner 20C to save the pulse reading 46Q to the patient's or potential patient's 12A electronic medical file. The software application 26A automatically tags the pulse reading 46Q with the date 72 and current time 74.

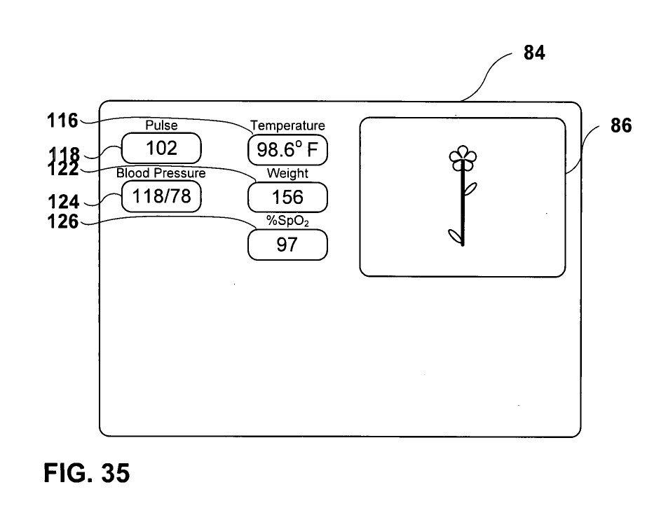

The pulse reading 46Q appears in window 118 on the health service practitioner's 20C diagnostic, display and control means 24A as shown in FIG. 35.

The reading of the percent of hemoglobin that is saturated with oxygen 46Y appears in window 126 on the health service practitioner's 20C diagnostic, display and control means 24A as shown in FIG. 35.

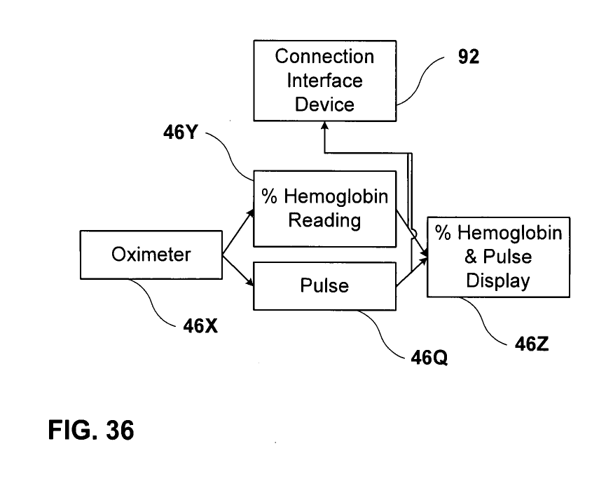

FIG. 36 shows the pulse 46Q and the percent of hemoglobin that is saturated with oxygen 46Y fed to the connection interface device 92 for connection 90 to the device or terminal 16.

Electrocardiogram

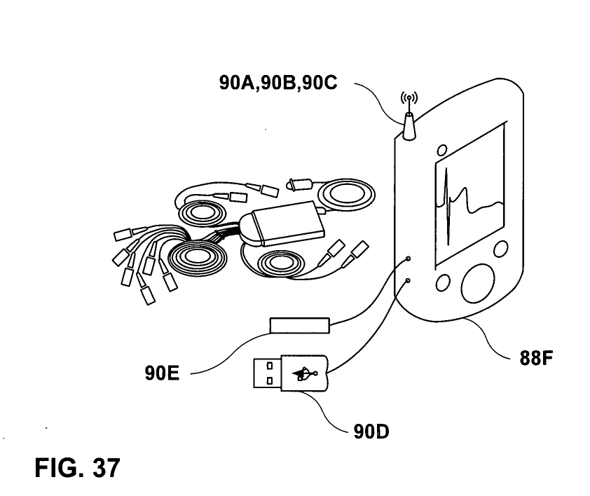

The sixth data device 88 is a device for obtaining an electrocardiograph, such as an electrocardiogram unit 88F. Numbers of electrocardiogram units 88F are commercially available from Biolog®, Bionet®, Burdich®, Brentwood®, Cardioline®, GE Marquette®, Midmark®, Nihon Kohden®, Phillips®, QRS®, Schiller America®, WelchAllyn® and others. A preferred embodiment of an electrocardiogram unit 88F to be deployed in the Remote Diagnostic & Treatment System 10A is shown in FIG. 37, and includes a connection 90 to a device or terminal 16. The embodiment shown in FIG. 37 includes one or more wireless connections, Bluetooth 90A, WiFi 90B and UWB 90C, and one or more wired connections, USB 90D and FireWire 90E.

For a cellular or PCS wireless phone 16B to receive the electrocardiogram and pulse reading from the electrocardiogram unit 88F, it must have embedded software 18A that recognizes that the electrocardiogram and pulse reading are being sent to the wireless phone 16B. Having received the electrocardiogram and pulse reading from the electrocardiogram unit 88F, there must be additional software 18A to forward the electrocardiogram and pulse reading to the diagnostic, display and control means 24A used by a health service practitioner 20C.

Similarly, the diagnostic, display and control software application 26A deployed on a diagnostic, display and control means 24A used by a health service practitioner 20C has to have the ability to display the electrocardiogram and pulse reading received from the cellular or PCS wireless phone 16B. The health service practitioner 20C must have the ability to take or retake the electrocardiogram and pulse reading.

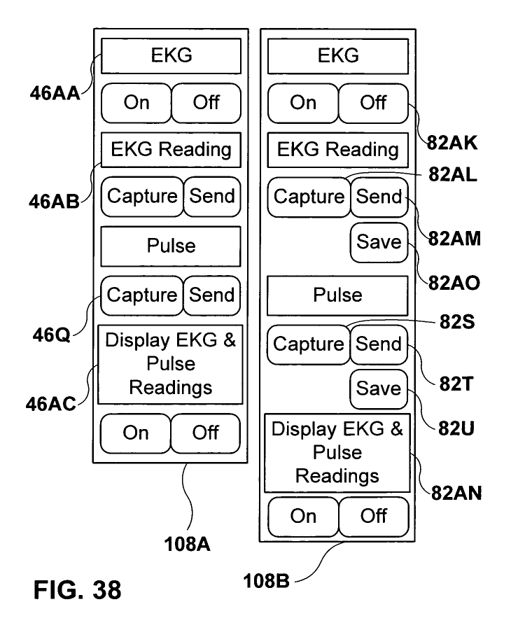

FIG. 38 shows a sixth embodiment of a functional block diagram 108 of embedded software 18A that enables remote functional control of the data devices 88 for the diagnostic means 16A as well as a software application 26A that enables remote functional control of the data devices 88 connected to the diagnostic means 16A. The functions shown in 108A are deployed as a component of 18A, the software embedded in a cellular and PCS wireless phone 16B; those in 108B as a component of the diagnostic, display and control software application 26A.

The functions 46 embedded in a cellular or PCS wireless phone 16B for controlling the data device 88 the electrocardiogram 88F include, but are not limited to:
Turn the electrocardiogram unit 88F on or off 46AA;
Capture and send the electrocardiogram reading 46AB;
Capture and send the pulse reading 36Q; and
Turn the electrocardiogram reading display 46AC on or off.

The functional control means 82 in the diagnostic, display and control software application 26A for remotely controlling the electrocardiogram unit 88F via a cellular or PCS wireless phone 16B include, but are not limited to:
An “off” button and an “on” button 82AK for controlling 46AA the electrocardiogram unit 88F;
A button 82AL for capturing or recapturing the electrocardiogram reading 46AB;
A button 82AM for sending the electrocardiogram reading 46AA to a health service practitioner 14;
A button 82S for capturing or recapturing the pulse reading 46Q;
A button 82T for sending the pulse reading 46Q to a health service practitioner 14; and
An “off” button and an “on” button 82AN for controlling the display 46AC of the electrocardiogram reading.

An additional functional control means 82AO allows the health service practitioner 20C to save the electrocardiogram reading 46AA to the patient's or potential patient's 12A electronic medical file. The software application 26A automatically tags the electrocardiogram 46AA with the date 72 and current time 74.

An additional functional control means 82U allows the health service practitioner 20C to save the pulse reading 46Q to the patient's or potential patient's 12A electronic medical file. The software application 26A automatically tags the pulse reading 46Q with the date 72 and current time 74.

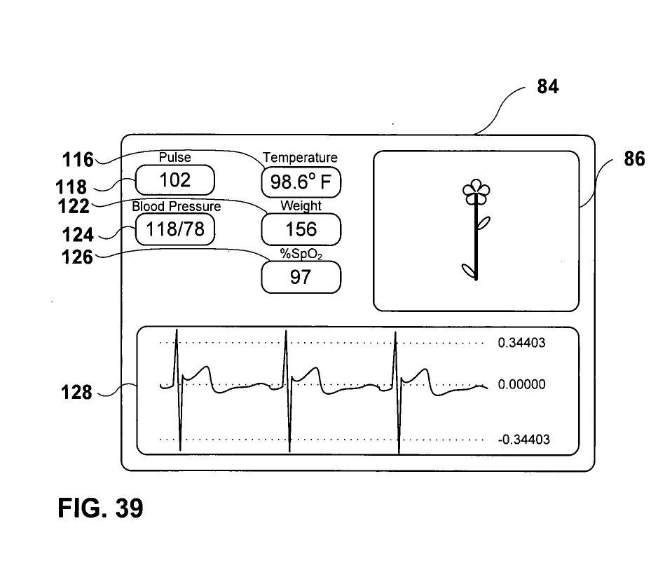

The pulse reading 46Q appears in window 118 on the health service practitioner's 20C diagnostic, display and control means 24A as shown in FIG. 39.

The electrocardiogram 46AA appears in window 128 on the health service practitioner's 20C diagnostic, display and control means 24A as shown in FIG. 39.

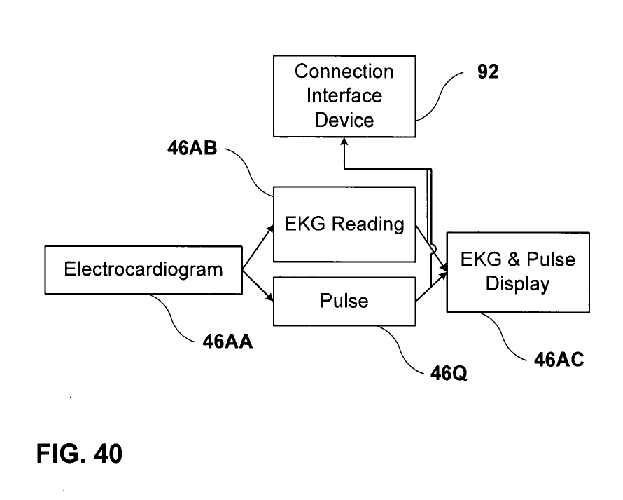

FIG. 40 shows the pulse 46Q and the electrocardiogram 46AB fed to the connection interface device 92 for connection 90 to the device or terminal 16.

Glucose Meter

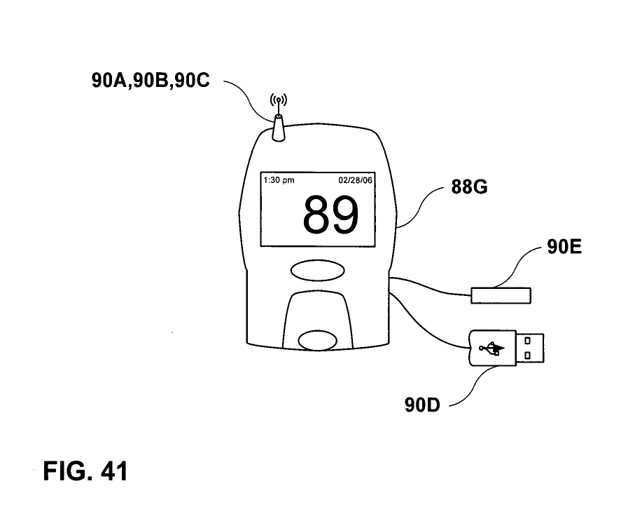

The seventh data device 88 is a device for measuring the glucose level in the blood, such as a glucose meter 88G. Numbers of glucose meters 80G are commercially available from Ascensia®, BD Logic®, Home Diagnostics, Inc.®, Hypoguard®, LifeScan®, MediSense®, Roche Diagnostics®, SpectRx, Inc.® and others. A preferred embodiment of a glucose meter 80G to be deployed in the Remote Diagnostic & Treatment System 10A is shown in FIG. 41, and includes a connection 90 to a device or terminal 16. The embodiment shown in FIG. 41 includes one or more wireless connections, Bluetooth 90A, WiFi 90B and UWB 90C, and one or more wired connections, USB 90D and FireWire 90E.

For a cellular or PCS wireless phone 16B to receive the glucose reading from the meter 88G, it must have embedded software 18A that recognizes that a digital weight reading is being sent to the wireless phone 16B. One embodiment of the embedded software 18A allows the glucose reading to be displayed on the screen 34A of the wireless phone 16B. Having received the glucose reading from the meter 88G, there must be additional software 18A to forward the glucose reading to the diagnostic, display and control means 24A used by a health service practitioner 20C.

Similarly, the diagnostic, display and control software application 26A deployed on a diagnostic, display and control means 24A used by a health service practitioner 20C has to have the ability to display the glucose reading received from the cellular or PCS wireless phone 16B. The health service practitioner 20C must have the ability to take or retake the glucose reading, as well as to determine whether to display the glucose reading on the cellular or PCS wireless phone 16B.

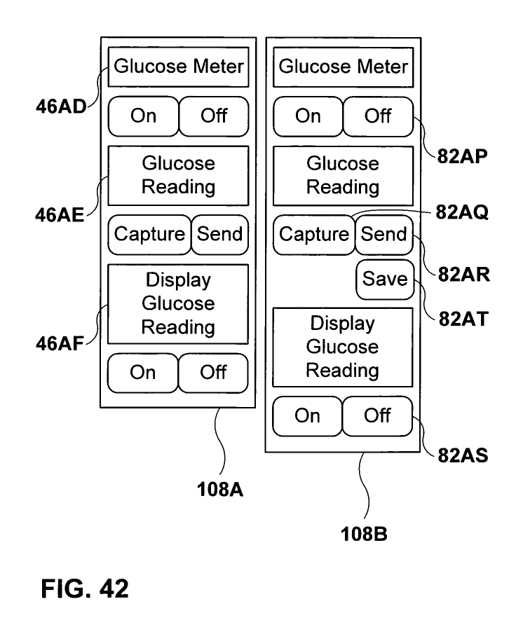

FIG. 42 shows a seventh embodiment of a functional block diagram 108 of embedded software 18A that enables remote functional control of the data devices 88 for the diagnostic means 16A as well as a software application 26A that enables remote functional control of the data devices 88 connected to the diagnostic means 16A. The functions shown in 108A are deployed as a component of 18A, the software embedded in a cellular and PCS wireless phone 16B; those in 108B as a component of the diagnostic, display and control software application 26A.

The functions 46 embedded in a cellular or PCS wireless phone 16B for controlling the data device 88 the glucose meter 88F include, but are not limited to:
Turn the glucose meter 88G on or off 46AD;
Capture and send the glucose reading 46AE; and
Turn the glucose reading display 46AF on or off.

The functional control means 82 in the diagnostic, display and control software application 26A for remotely controlling the glucose meter 88G via a cellular or PCS wireless phone 16B include, but are not limited to:
An “off” button and an “on” button 82AP for controlling 46AD the glucose meter 80G;
A button 82AQ for capturing or recapturing the glucose reading 46AE;
A button 82AR for sending the glucose reading 46AD to a health service practitioner 20C; and
An “off” button and an “on” button 82AS for controlling the display 46AF of the glucose reading 46AE.

An additional functional control means 82AT allows the health service practitioner 20C to save the glucose reading 46AE to the patient's or potential patient's 12A electronic medical file. The software application 26A automatically tags the glucose reading 46AE with the date 72 and current time 74.

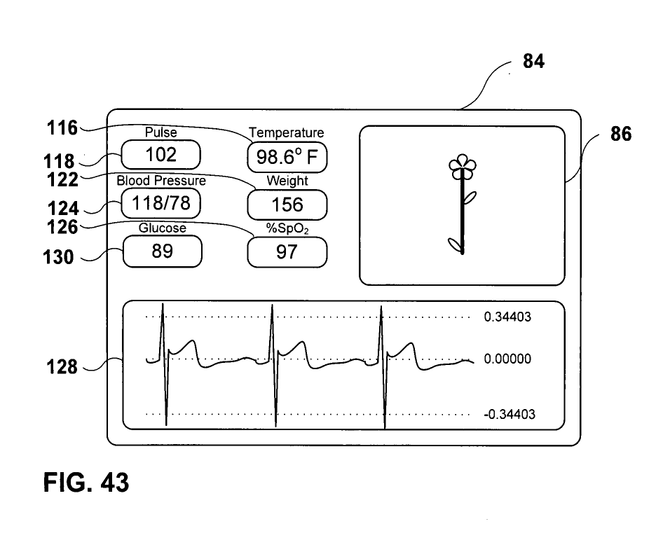

The glucose reading 46AE appears in window 130 on the health service practitioner's 20C diagnostic, display and control means 24A as shown in FIG. 43.

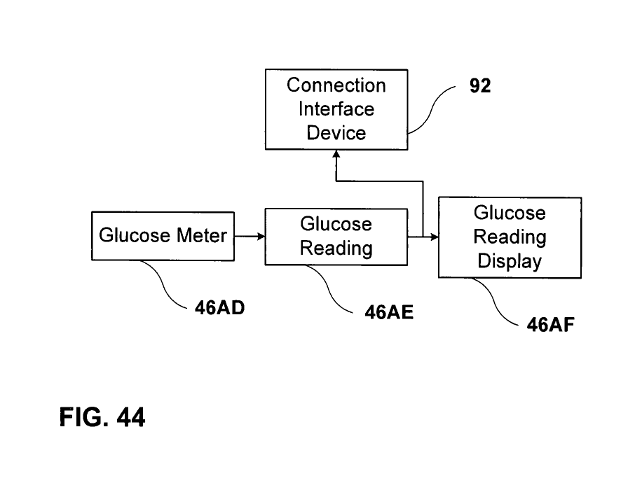

FIG. 44 shows the glucose reading 46AE fed to the connection interface device 92 for connection 90 to the device or terminal 16.

Otoscope

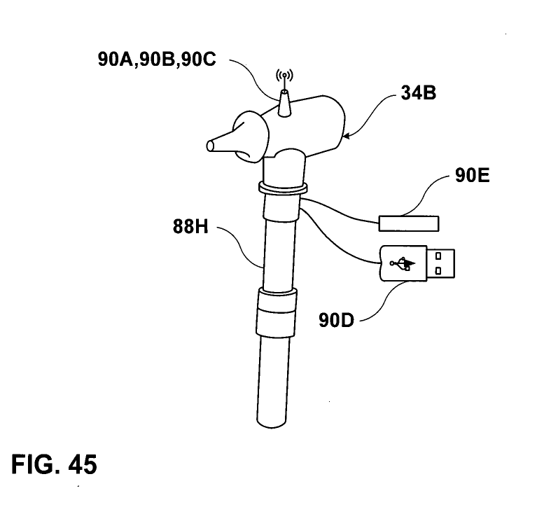

An eighth data device 88 is an otoscope 88H. An otoscope 88H is used to examine the ears, nose, and mouth. It contains a light and a magnifying lens. Numbers of otoscopes 88H are commercially available from American Diagnostic Corporation (ADC)®, Dr. Mom®, Heine®, Riester®, WelchAllyn® and others. A preferred embodiment of an otoscope 88H to be deployed in the Remote Diagnostic & Treatment System 10A is shown in FIG. 45, and includes a connection 90 to a device or terminal 16. The embodiment shown in FIG. 45 includes one or more wireless connections, Bluetooth 90A, WiFi 90B and UWB 90C, and one or more wired connections, USB 90D and FireWire 90E.

An otoscope is basically a visual aid to a health service practitioner 20C who has physical access to a patient or potential patient 12A. A camera 34B must be added to the otoscope 88H for it to be deployed in the Remote Diagnostic & Treatment System 10. The otoscope 88H must also have an interface that captures the images 38 and makes those images available to the connection 90 to the device or terminal 16.

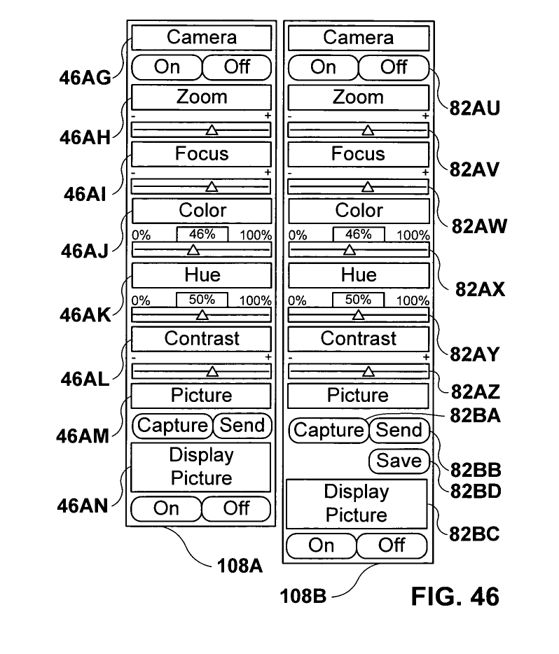

FIG. 46 shows a eighth embodiment of a functional block diagram 108 of embedded software 18A that enables remote functional control of the data devices 88 for the diagnostic means 16A as well as a software application 26A that enables remote functional control of the data devices 88 connected to the diagnostic means 16A. The functions shown in 108A are deployed as a component of 18A, the software embedded in a cellular and PCS wireless phone 16B; those in 108B as a component of the diagnostic, display and control software application 26A.

The functions 46 embedded in a cellular or PCS wireless phone 16B for controlling the data device 88 the otoscope 88H include, but are not limited to:
Turn the camera 88H on or off 46AG;
Zoom 46AH the camera 88H;
Focus 46AI the camera 88H;
Adjust the camera 88H color 46AJ;
Adjust the camera 88H hue 46AK;
Adjust the camera 88H contrast 46AL;
Take 46AM a picture 36; and
Turn the camera 88H display 46AN on or off.

The functional control means 82 in the diagnostic, display and control software application 26A for remotely controlling the stethoscope or high fidelity microphone 88B via a cellular or PCS wireless phone 16B include, but are not limited to:
An “off” button and an “on” button 82AU for controlling 46AG the camera 88H;
A slider 82AV that adjusts the zoom 46AH from zero percent (0%) to one hundred percent (100%);
A slider 82AW that adjusts the camera focus 46AI from minus (−) to plus (+);
A slider 82AX that adjusts the color 46AJ from zero percent (0%) to one hundred percent (100%);
A slider 82AY that adjusts the hue 46AK from zero percent (0%) to one hundred percent (100%);
A slider 82AZ that adjusts the contrast 46AL from minus (−) to plus (+);
A button 82BA for taking 46AM a picture 36;
A button 82BB for sending 46AM the picture 36; and
An “off” button and an “on” button 82BC for controlling the display 46AN of the camera 88H.

An additional functional control means 82BD allows the health service practitioner 20C to save the picture 42 to the patient's or potential patient's 12A electronic medical file. The software application 26A automatically tags the picture 42 with the date 72 and current time 74.

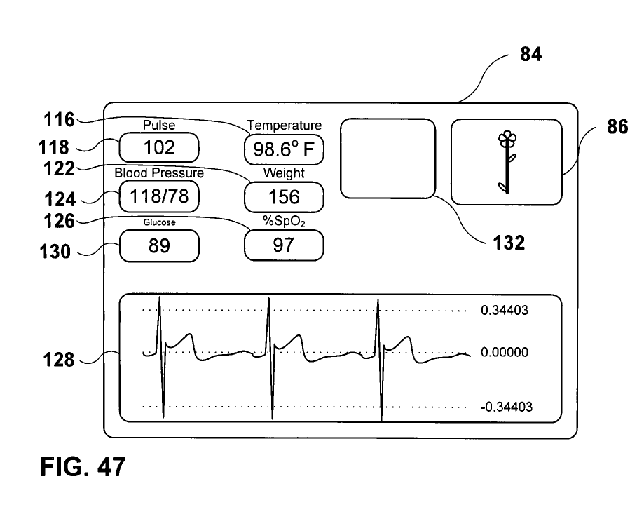

The picture 42 appears in window 132 on the health service practitioner's 20C diagnostic, display and control means 24A as shown in FIG. 47.

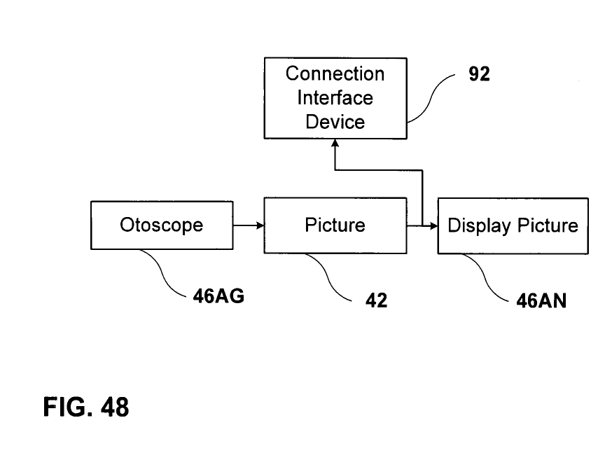

FIG. 48 shows the picture 42 fed to the connection interface device 92 for connection 90 to the device or terminal 16.

Ultrasound

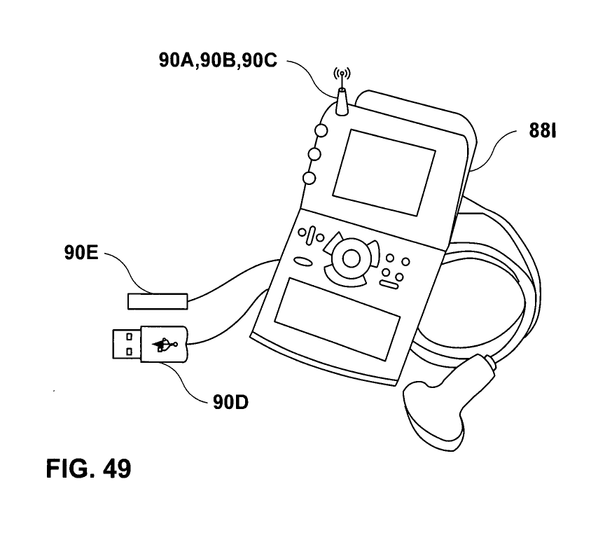

The ninth data device 88 is an ultrasound unit 88I. Numbers of ultrasound units 88I are commercially available from Amrex®, Intelect®, GE Logiq®, Koality®, Mettler®, Siemens Acuson® and others. A preferred embodiment of an ultrasound unit 88I to be deployed in the Remote Diagnostic & Treatment System 10A is shown in FIG. 49, and includes a connection 90 to a device or terminal 16. The embodiment shown in FIG. 49 includes one or more wireless connections, Bluetooth 90A, WiFi 90B and UWB 90C, and one or more wired connections, USB 90D and FireWire 90E.

For a cellular or PCS wireless phone 16B to receive the ultrasound readings from the ultrasound unit 88I, it must have embedded software 18A that recognizes that the ultrasound readings are being sent to the wireless phone 16B. Having received the ultrasound readings from the ultrasound unit 88I, there must be additional software 18A to forward the ultrasound readings to the diagnostic, display and control means 24 used by a health service practitioner 20C.

Similarly, the diagnostic, display and control software application 26A deployed on a diagnostic, display and control means 24A used by a health service practitioner 20C has to have the ability to display the ultrasound readings received from the cellular or PCS wireless phone 16B. The health service practitioner 20C must have the ability to take or retake the ultrasound readings.

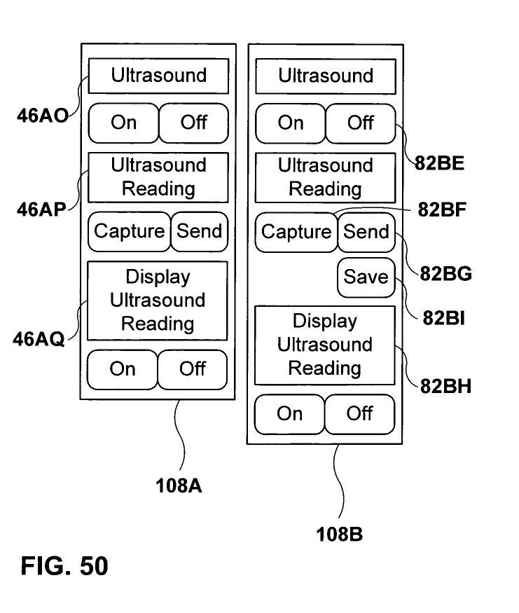

FIG. 50 shows a ninth embodiment of a functional block diagram 108 of embedded software 18A that enables remote functional control of the data devices 88 for the diagnostic means 16A as well as a software application 26A that enables remote functional control of the data devices 88 connected to the diagnostic means 16A. The functions shown in 108A are deployed as a component of 18A, the software embedded in a cellular and PCS wireless phone 16B; those in 108B as a component of the diagnostic, display and control software application 26A.

The functions 46 embedded in a cellular or PCS wireless phone 16B for controlling the data device 88 the ultrasound unit 88I include, but are not limited to:
Turn the ultrasound unit 88I on or off 46AO;
Capture and send the ultrasound reading 46AP; and
Turn the ultrasound display 46AQ on or off.

The functional control means 82 in the diagnostic, display and control software application 26A for remotely controlling the ultrasound unit 88I via a cellular or PCS wireless phone 16B include, but are not limited to:
An “off” button and an “on” button 82BE for controlling 46AO the ultrasound unit 88I;
A button 82BF for capturing or recapturing the ultrasound readings 46AP;
A button 82BG for sending the ultrasound readings 46AP to a health service practitioner 20C; and
An “off” button and an “on” button 82BH for controlling the display 46AQ of the ultrasound 88I.

An additional functional control means 82BI allows the health service practitioner 20C to save the ultrasound readings 46AN to the patient's or potential patient's 12A electronic medical file. The software application 26A automatically tags the ultrasound readings 46AA with the date 72 and current time 74.

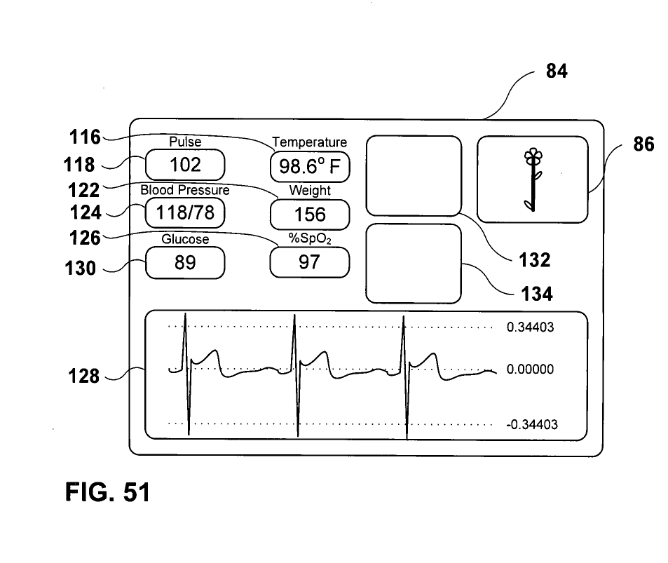

The ultrasound readings 46AP appears in window 134 on the health service practitioner's 20C diagnostic, display and control means 24A as shown in FIG. 51.

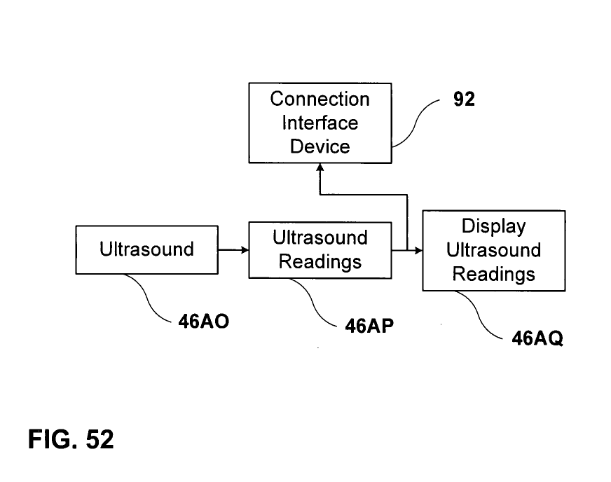

FIG. 52 shows the ultrasound readings 46AP fed to the connection interface device 92 for connection 90 to a device or terminal 16.

External Camera

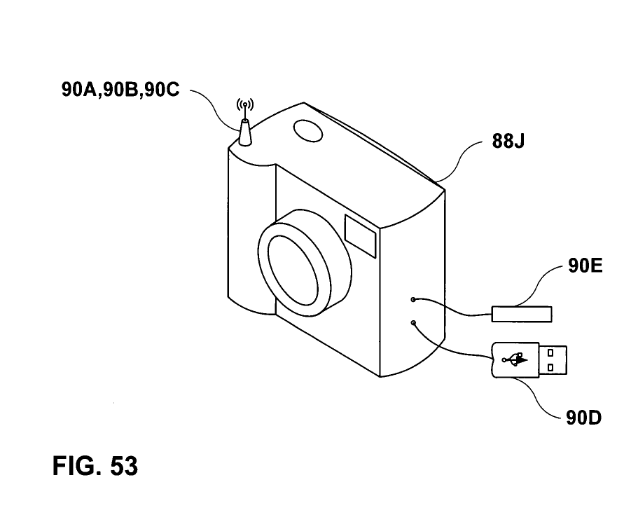

The tenth data device 88 is a camera 88J that is not part of a diagnostic means 16A. A preferred embodiment of an external camera 88J to be deployed in the Remote Diagnostic & Treatment System 10 is shown in FIG. 53, and includes a connection 90 to a device or terminal 16. The embodiment shown in FIG. 53 includes one or more wireless connections, Bluetooth 90A, WiFi 90B and UWB 90C, and one or more wired connections, USB 90D and FireWire 90E.

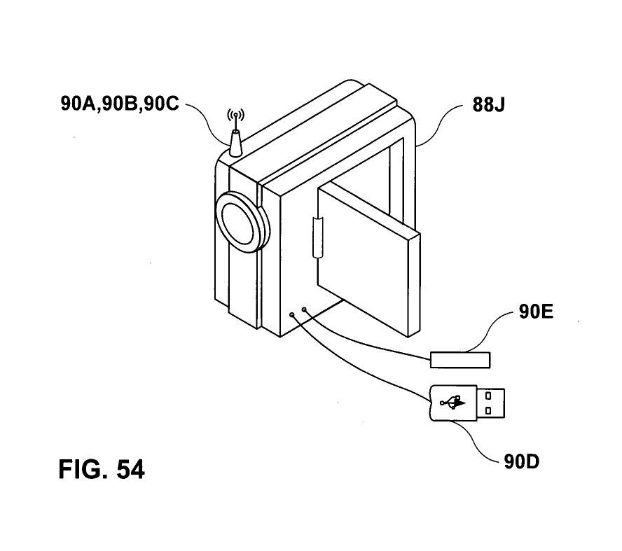

The specific embodiment of the external camera 88J shown in FIG. 53 is a digital camera. Modern digital cameras take pictures 42 as well as short videos 136. An alternative embodiment of the external camera 88J is a digital video recorder as shown in FIG. 54. Modern digital video cameras take videos 136 as well as pictures 42. The embodiment shown in FIG. 53 includes one or more wireless connections, Bluetooth 90A, WiFi 90B and UWB 90C, and one or more wired connections, USB 90D and FireWire 90E.

At present there are only few manufacturers of Bluetooth-enabled cameras, Concord Camera®, Panasonic®, Sony®, Sony Ericcson®; other manufacturers are expected to enter this market in the near future. Some manufacturers have WiFi-enabled cameras, 4xem®, Axis Communications®, BenQ®, Creative Labs®, D-Link®, Kodak®, Linksys®, Nikon®, Sony® and others. Additional manufacturers are expected to enter this market in the near future. It is expected that manufacturers will add UWB to cameras in the near future.

For a cellular or PCS wireless phone 16B to receive the image from the external camera 88J, it must have embedded software 18A that recognizes that a camera image is being sent to the wireless phone 16B. One embodiment of the embedded software 18A allows the camera image to be displayed on the screen 40A of the wireless phone 16B. Having received the image from the external camera 88J, there must be additional software 18A to forward the external camera image to the diagnostic, display and control means 24A used by a health service practitioner 20C.

Similarly, the diagnostic, display and control software application 26A deployed on a diagnostic, display and control means 24A used by a health service practitioner 20C has to have the ability to display the external camera image received from the cellular or PCS wireless phone 16B. The health service practitioner 20C must have the ability to take or retake the camera images, as well as to determine whether to display the external camera image on the cellular or PCS wireless phone 16B.

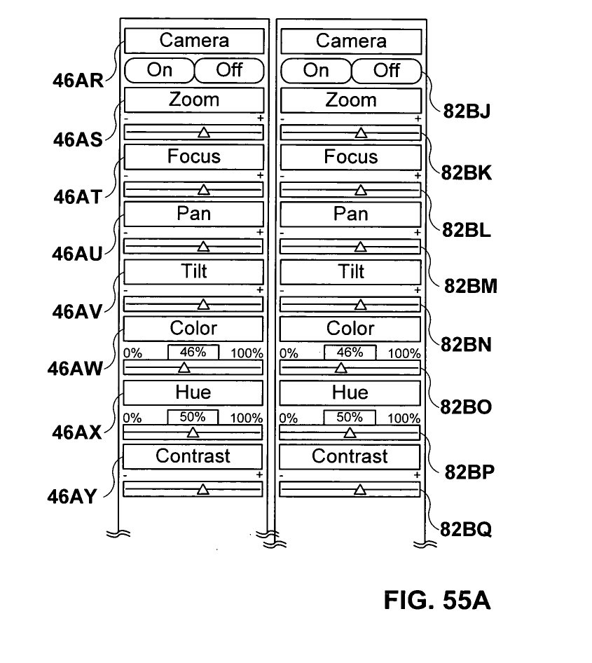

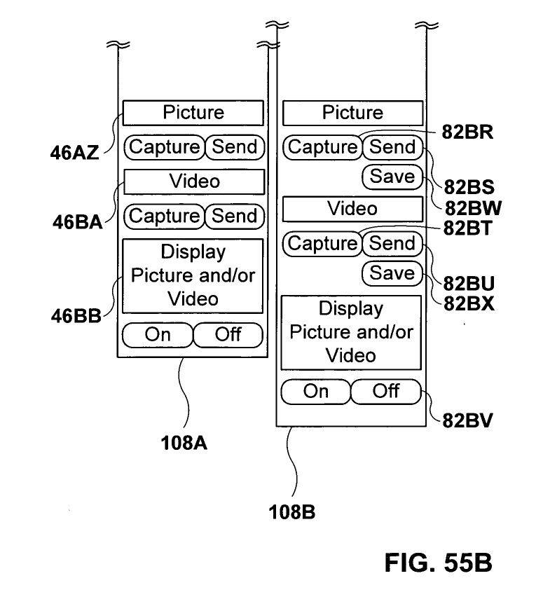

FIG. 55 shows a tenth embodiment of a functional block diagram 108 of embedded software 18A that enables remote functional control of the data devices 88 for the diagnostic means 16A as well as a software application 26A that enables remote functional control of the data devices 88 connected to the diagnostic means 16A. The functions shown in 108A are deployed as a component of 18A, the software embedded in a cellular and PCS wireless phone 16B; those in 108B as a component of the diagnostic, display and control software application 26A.

The functions 46 embedded in a cellular or PCS wireless phone 16B for controlling the data device 88 the external camera 88J include, but are not limited to:
Turn the camera 88J on or off 46AR;
Zoom 46AS the camera 88J;
Focus 46AT the camera 88J;
Pan 46AU camera 88J;
Tilt 46AV camera 88J;
Adjust the camera 88J color 46AW;
Adjust the camera 88J hue 46AX;
Adjust the camera 88J contrast 46AY;
Take 46AZ a picture 36;
Take 46BA video 112; and
Turn the camera 88J display 46BB on or off.

The functional control means 82 in the diagnostic, display and control software application 26A for remotely controlling the external camera 88J via a cellular or PCS wireless phone 16B include, but are not limited to:
An “off” button and an “on” button 82BJ for controlling 46AR the camera 88J;
A slider 82BK that adjusts the zoom 46AS from zero percent (0%) to one hundred percent (100%);
A slider 82BL that adjusts the camera focus 46AT from minus (−) to plus (+);
A slider 82BM that adjusts the camera pan 46AU from minus (−) to plus (+);
A slider 82BN that adjusts the camera tilt 46AV from minus (−) to plus (+);
A slider 82BO that adjusts the color 46AW from zero percent (0%) to one hundred percent (100%);
A slider 82BP that adjusts the hue 46AX from zero percent (0%) to one hundred percent (100%);
A slider 82BQ that adjusts the contrast 46AY from minus (−) to plus (+);
A button 82BR for taking 46AZ a picture 38;
A button 82BS for sending 46BA the picture 38;
A button 82BT for capturing 46BB video 130;
A button 82BU for sending 46BC video 130; and
An “off” button and an “on” button 82BV for controlling the display 46BB of the camera 88J.

An additional functional control means 82BW allows the health service practitioner 20C to save the picture 42 to the patient's or potential patient's 12A electronic medical file. The software application 26A automatically tags the picture 42 with the date 72 and current time 74.

An additional functional control means 82BX allows the health service practitioner 20C to save the video 136 to the patient's or potential patient's 12A electronic medical file. The software application 26A automatically tags the video 136 with the date 72 and current time 74.

The external camera 88J picture 42 appears in window 132 on the health service practitioner's 20C diagnostic, display and control means 24A as shown in FIG. 51.

The external camera 88J video 136 appears in window 134 on the health service practitioner's 20C diagnostic, display and control means 24A as shown in FIG. 51.

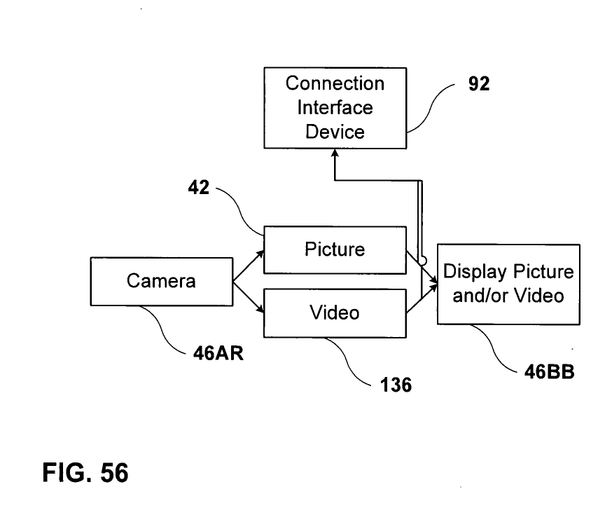

FIG. 56 shows the picture 42 or the video 136 fed to the connection interface device 92 for connection 90 to a device or terminal 16.

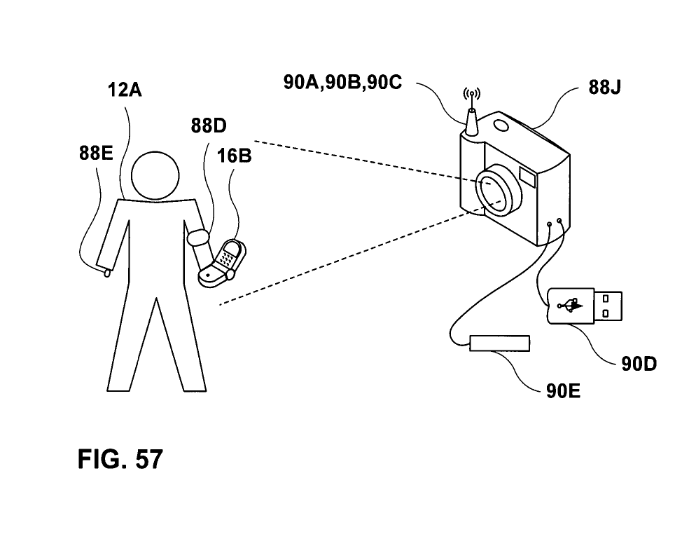

The external camera 88J is particularly useful to the health service practitioner 20C for observing the patient or potential patient 12A as he or she utilizes the diagnostic means 16A, including data devices 88, as shown in FIG. 57. In this embodiment the external camera 88J is set away from the patient or potential patient 12A so that the health service practitioner 20C can see what the patient or potential patient 12A is doing, especially in placing data devices 88 on his or her body.

III. Alternative Embodiments of the Invention.

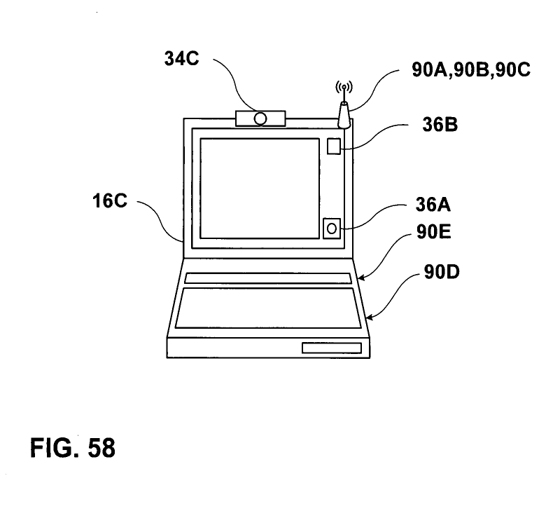

An embodiment of the device or terminal 16 has thus far been described as a cellular or PCS wireless phone 16B. A first alternative embodiment of the device or terminal 16 is a Personal Computer (PC) equipped with a camera 28, as shown in FIG. 58. The preferred embodiment shown in FIG. 56 shows a laptop PC 16C with a built in camera 28C. Data devices 88 may be connected 90 to the laptop PC 16C using wires or wirelessly. Modern day laptops 16C have Bluetooth 90A and WiFi 90B capabilities built in. It is expected that in the near future they may also have UWB 90C built in. Today, all laptop PCs 16C have at least one USB port 90D and at least one FireWire port 90E.

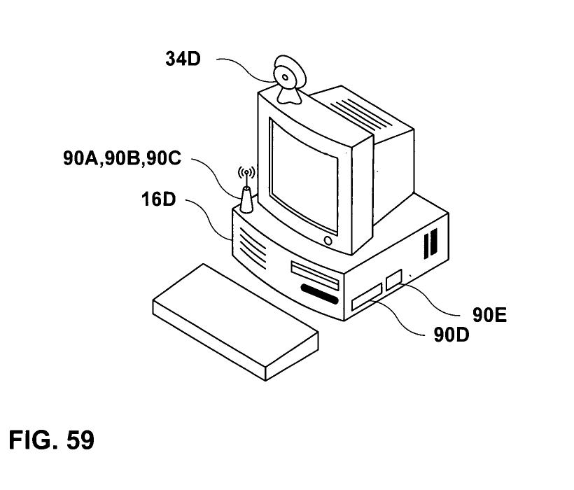

FIG. 59 shows a second alternative embodiment of a PC, a desktop computer 16D with an attached camera 28D. The desktop computer 16D shown in FIG. 59 is enabled with Bluetooth 90A and WiFi 90B capabilities. It is expected that in the near future they may also have UWB 90C built in. Today, all desktop PCs 16D have at least one USB port 90D and at least one FireWire port 90E.

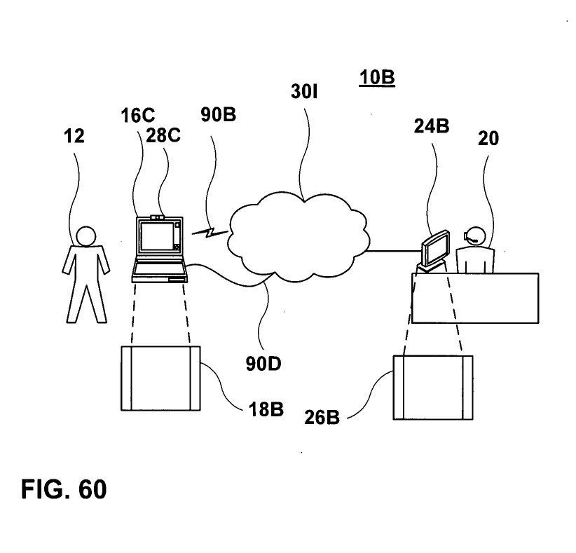

FIG. 60 shows an alternative embodiment of the Remote Diagnostic & Treatment System 10 in which the device or terminal is a laptop PC 16C. In this embodiment of the Remote Diagnostic & Treatment System 10B the laptop PC 16C is connected 90 to the network 30, specifically the Internet 30I, using a wired USB 90D or a wireless connection, specifically a WiFi connection 90B. Additionally, in this embodiment the embedded software that enables remote function control 18B and the diagnostic, display and control means 24B and software application(s) 26B are Internet-enabled.

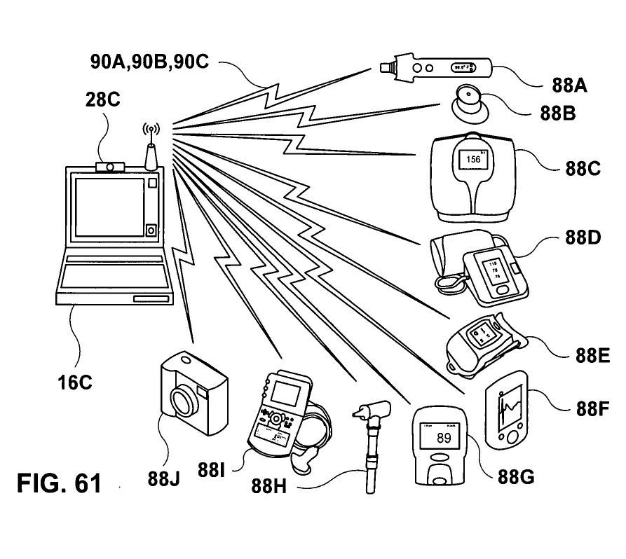

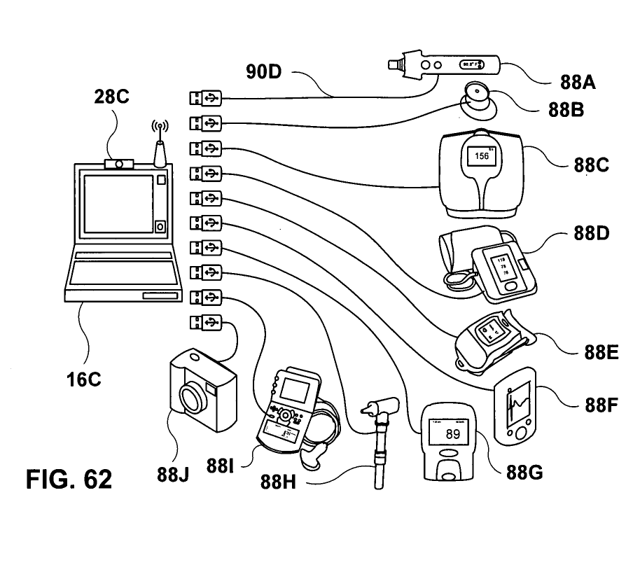

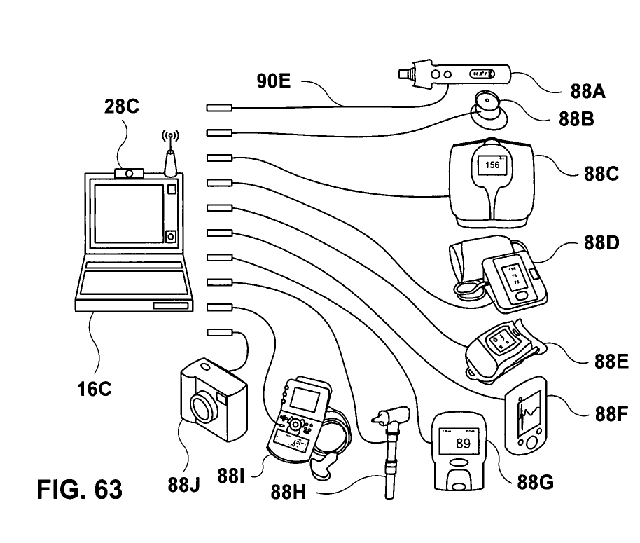

All of the data devices 88 may be connected to a laptop PC 16C or a desktop PC 16D via a wireless connection 90 as shown in FIG. 61, Bluetooth 90A, WiFi 90B or UWB 90C, or via a wired connection 90 as shown in FIG. 62 for USB 90D and in FIG. 63 for FireWire 90E.

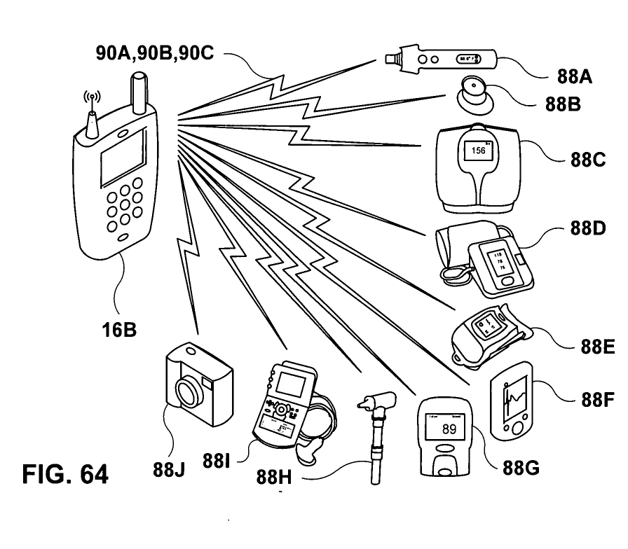

Many cellular and PCS wireless phones 16B are today enabled with Bluetooth 90A. Cellular and PCS wireless phones 16B enabled with WiFi 90B are just becoming available from Avaya®, E-TEN®, Hewlett-Packard (HP)®, Microsoft®, Motorola®, NEC®, Proxim® and others. Data devices 88 may be connected to these new WiFi-enabled cellular and PCS wireless phones 16B as shown in FIG. 64. In the future manufacturers may add UWB 90C chip sets to cellular and PCS wireless phones 16B.

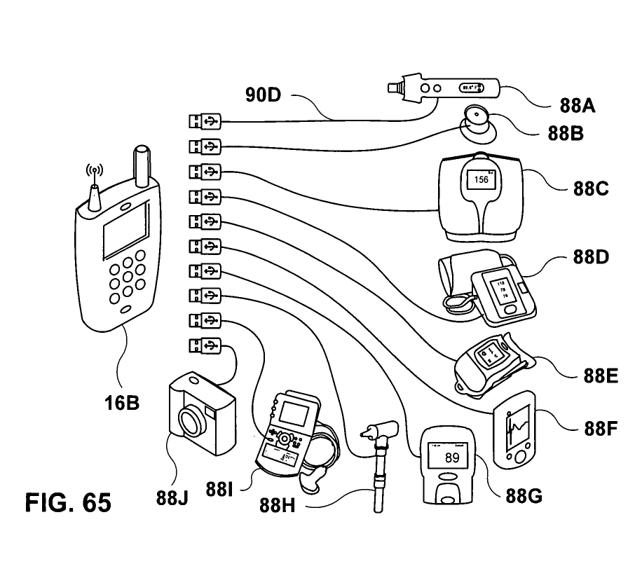

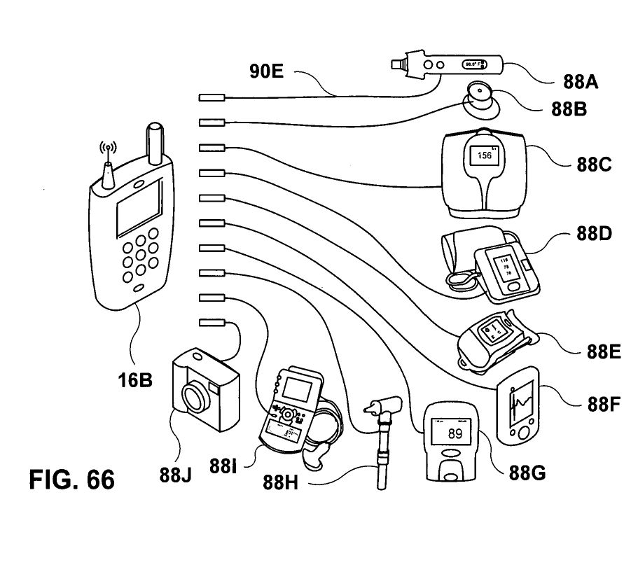

Most cellular and PCS wireless phones 16B have a data port. Today, these data ports are proprietary. In the future there is no reason for cellular and PCS wireless phones 16B not having USB 90D and/or FireWire 90E ports. FIG. 65 shows connection 90 of data devices 88 to a cellular or PCS wireless phone 16B via USB 90D; FIG. 66 shows connection 90 of data devices 88 to a cellular or PCS wireless phone 16B via FireWire 90E.

There are other developing and emerging wireless waveforms and network topologies that may be used in the Remote Diagnostic & Treatment System 10.

Some medical conditions require continuous or semi-continuous monitoring. In one embodiment a cellular or PCS wireless phone 16B can be left on and connected to a health service practitioner 20C and the images 42 or data from data devices 88 continuously transmitted to the health service practitioner 20C. Alternatively and more practically, the patient or potential patient 12A can save images 42 or data from data devices 88 in the cellular or PCS wireless phone 16B, laptop PC 16C or desktop PC 16D for transmission to a health service practitioner 20C on a scheduled or an ad hoc basis.

IV. Alternative Applications of the Remote Diagnostic & Treatment System

The Remote Diagnostic & Treatment System 10 has numbers of applications beyond remote triage and health service monitoring. A first alternative embodiment addresses remote triage and monitoring of elderly patients or potential patients 12A, particularly those in assisted living environments. Elderly patients or potential patients 12A in assisted living environments are often provided a lanyard-based or clothing clipped button device that the patient or potential patient 12A can push to alert the staff in the event of an emergency. Pushing the button usually turns on a light in a monitoring station and causes an attendant to go to the patient's or potential patient's 12A unit to assess the situation. More advanced versions of the “button” include a microphone that enables the patient or potential patient 12A to talk to the monitoring attendant.

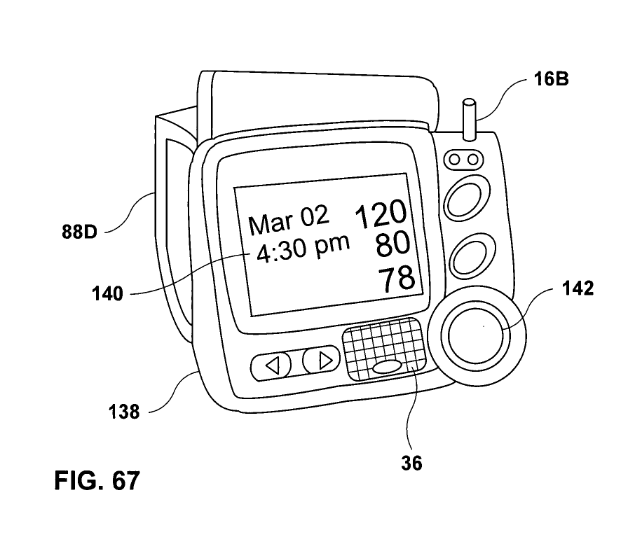

An embodiment of the Remote Diagnostic & Treatment System 10A to be deployed in an assisted living environment is shown in FIG. 67. In this embodiment an elderly patient or potential patient 12A that requires health monitoring wears a device 138 on his or her wrist that includes an embedded cellular or PCS wireless phone 16B with speaker phone 36 and one or more monitoring data devices 88. In the embodiment shown in FIG. 67 the data device is a blood pressure and pulse rate monitor 88D. The device 138 also functions as a watch 140. The device 138 also includes a button 142 that the elderly patient or potential patient 12A can push in the case of an emergency that has an emergency phone number programmed into the button's activation. The preprogrammed number might be “911” or some other emergency service number that connects to a health service practitioner 20C. When the emergency button 142 is pushed and the connection to the emergency phone number completed, the speaker phone 36 is turned on and the data from the data device 88 transmitted to the health service practitioner 20C. The health service practitioner 20C can talk to the patient or potential patient 12A as well as hear them and the conditions surrounding them. These capabilities can assist the health service practitioner 20C to respond to the emergency or incident.

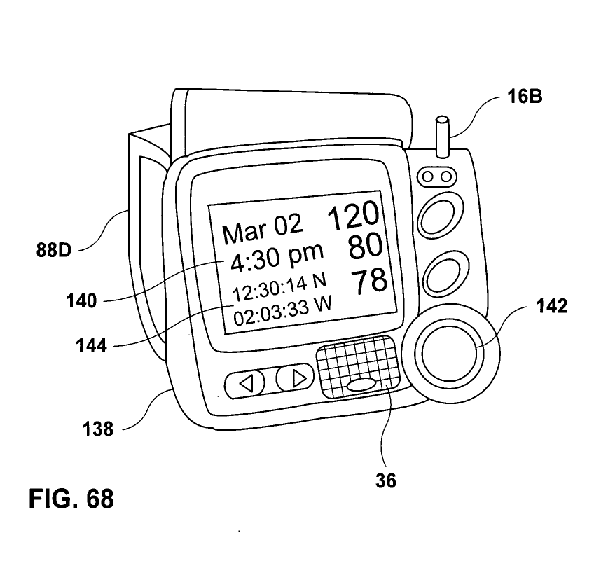

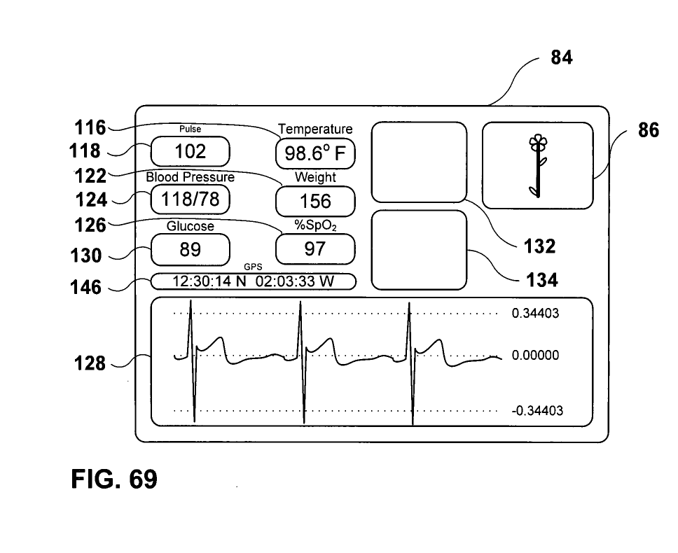

The same or similar device 138 can be worn by a soldier to monitor his or her health status as well as to provide remote triage if the soldier is injured. In this embodiment it is beneficial to also embed a Global Positioning System (GPS) receiver 144 into the device 138 as shown in FIG. 68 so that the health service practitioner 20C can know the location of the soldier. The diagnostic, display and control software application 26A deployed on the diagnostic, display and control means 24A has to receive and display the GPS 144 data as shown in FIG. 68. The GPS 144 location appears in window 146 on the health service practitioner's 20C diagnostic, display and control means 24A as shown in FIG. 69. In a preferred embodiment the display of the GPS 144 data is in the form of a map.

A device 138 with embedded GPS 144 is also useful for keeping track of patients or potential patients 12A with Alzheimer's or other dementia disablements.

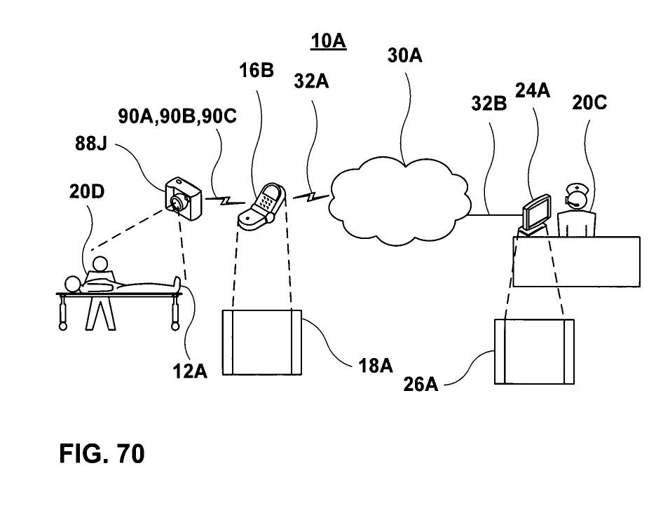

The Remote Diagnostic & Treatment System 10A may also be used to support other health service practitioners 20B. For example, devices or terminals 16 and data devices 88 may be deployed in ambulances and other emergency vehicles as shown in FIG. 70. In the embodiment shown in FIG. 70 a paramedic 20D consults with a remote health service practitioner 20C about the patient's or potential patient's 12A condition.

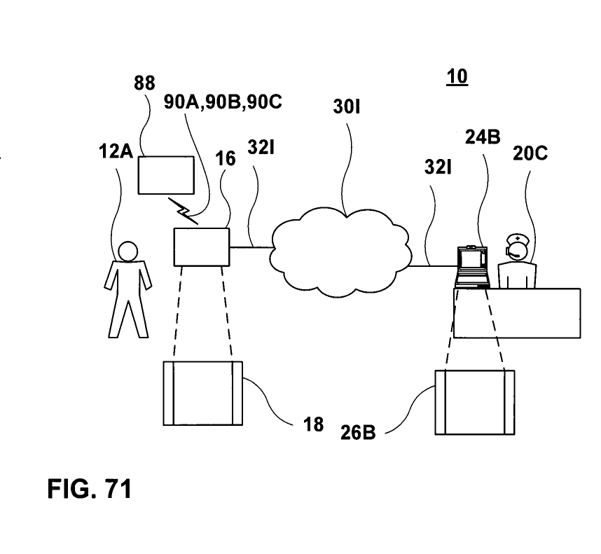

All of the embodiments described thus far have the health service practitioner 20C in a fixed location. The technologies of the Remote Diagnostic & Treatment System 10 enable the health service practitioner 20C to work from home or on the move. In the embodiment shown in FIG. 71 the health service practitioner 20C is at home with a laptop PC 16C as the diagnostic, display and control means 24B, and connected 32I to the Internet 30I. The diagnostic, display and control software application 26B is Internet-enabled.

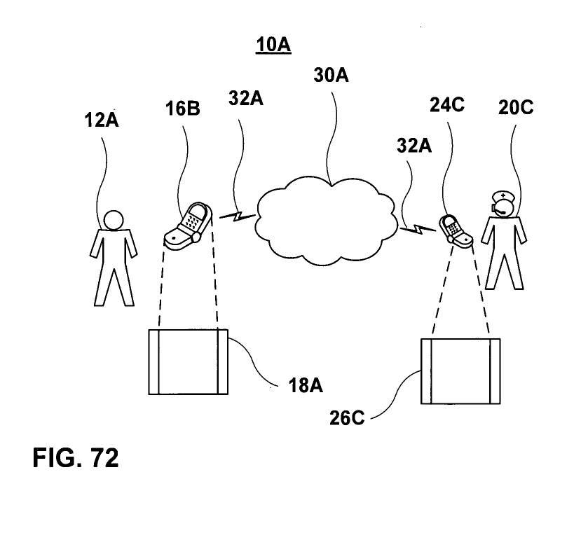

In the embodiment shown in FIG. 72 the health service practitioner 20C can be on the move. In this embodiment the diagnostic, display and control means 24C is a cellular or PCS wireless phone with the diagnostic, display and control software application 26C embedded into it.

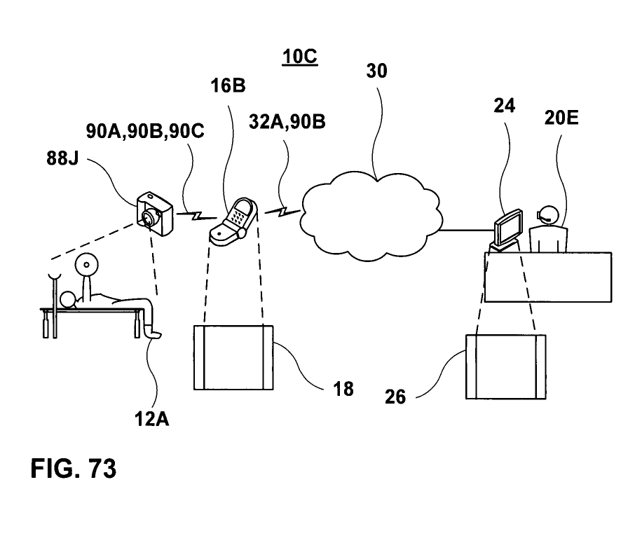

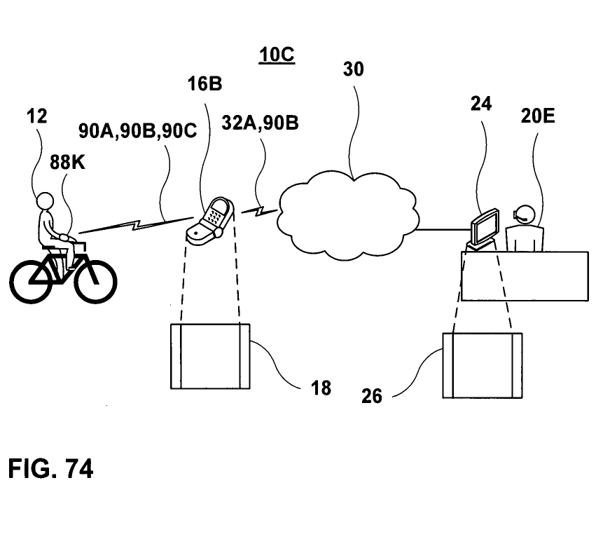

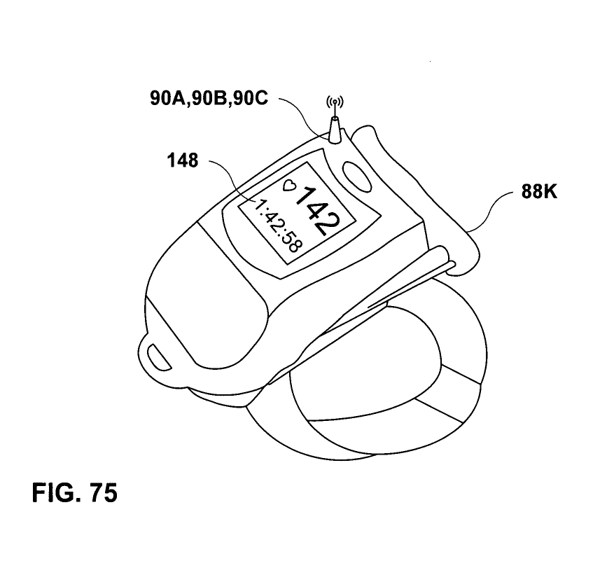

The Remote Diagnostic & Treatment System 10 may also be used for physical rehabilitation and athletic performance coaching. In this embodiment of the Remote Diagnostic & Treatment System 10C the health service practitioner 20C is replaced by a physical therapist or athletic coach 20E. A preferred embodiment of this application is shown in FIG. 73. An external camera 88J is deployed so the physical therapist or athletic coach 20E can observe the patient or potential patient 12A lifting weights. By turning on the speaker phone 36 the physical therapist or athletic coach 20E can correct the patient's or potential patient's 12A body position by speaking to him or her. The image from the external camera 88J can be transmitted to the cellular or PCS wireless phone 16B via Bluetooth 90A, WiFi 90B or UWB 90C. Similarly, the cellular or PCS wireless phone 16B may communicate with the network 32 using cellular or PCS frequencies 32A or WiFi 90B, if enabled. Although FIG. 73 shows the coaching in a fixed setting, there is no reason why the coaching cannot take place while moving, for example, on bicycle. In this embodiment, shown in FIG. 74, the patient or potential patient 12A wears a heart rate monitor 88K that communicates with to the cellular or PCS wireless phone 16B via Bluetooth 90A, WiFi 90B or UWB 90C as shown in FIG. 75. Heart rate monitors are commercially available from Acumen®, Cardiosport®, Mio®, Polar®, Reebok® and others, and often include watches 140 or stop watches 148.

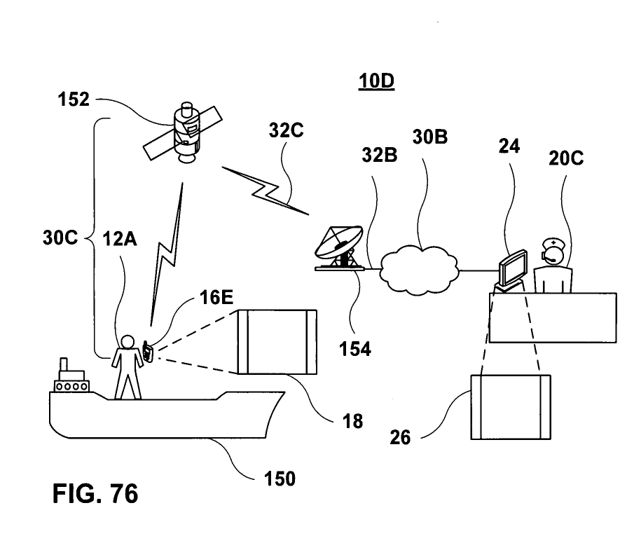

An additional embodiment of the Remote Diagnostic & Treatment System 10D is shown in FIG. 76. In this embodiment a patient or potential patient 12A in a remote location, in this embodiment aboard a ship 150, uses a satellite phone 16E as a diagnostic means 16A. The satellite phone 16E connects 32C to a satellite 152 and then to an earth station 154 connected 32B to the PSTN 30B. Satellite network 30C capacity is available from Iridium®, Globalstar®, Inmarsat®, New Skies®, Intelsat® and others.

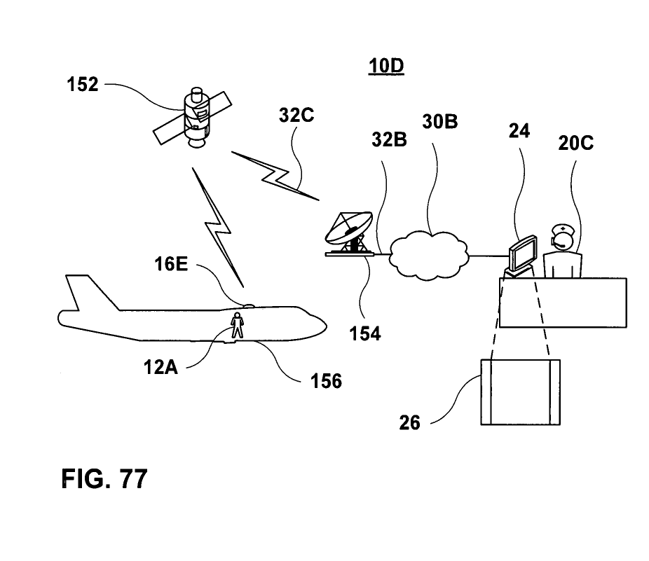

An additional embodiment of the Remote Diagnostic & Treatment System 10D is shown in FIG. 77. In this embodiment a patient or potential patient 12A aboard an airplane 156, uses an aircraft satellite phone 16F to connect 32C to a satellite 152 and then to an earth station 154 connected 20B to the PSTN 30B. Aircraft satellite phone services are available from Inmarsat®, New Skies®, Intelsat® and Connection®.

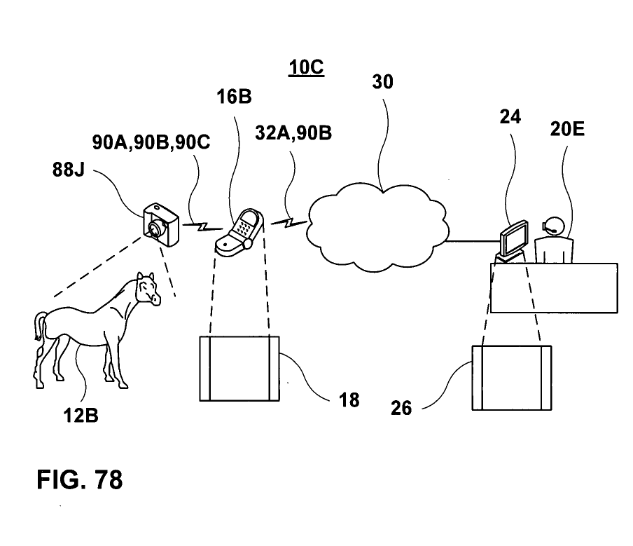

Although described thus far in human terms, the Remote Diagnostic & Treatment System 10 may also be used to treat animals. In the embodiment shown in FIG. 78 the “patient” is an animal 12B, here a horse, and the health service practitioner is a veterinarian 20E.

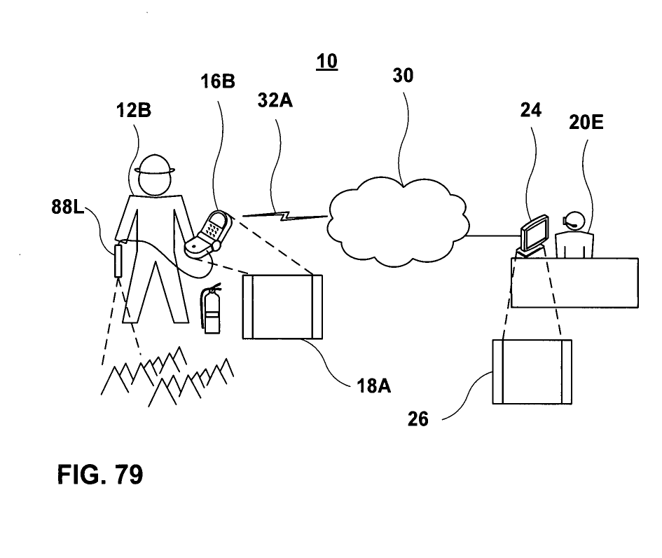

Although described thus far in health care terms, embodiments of the Remote Diagnostic & Treatment System 10 may be used for consultations between field personnel and others. For example, in the embodiment shown in FIG. 79 a fire investigator 12C is using a chemical sniffer 88L attached to a cellular or PCS wireless phone 16B to get data about the potential causes of a fire. The data is transmitted to a laboratory technician 20F for assessment.

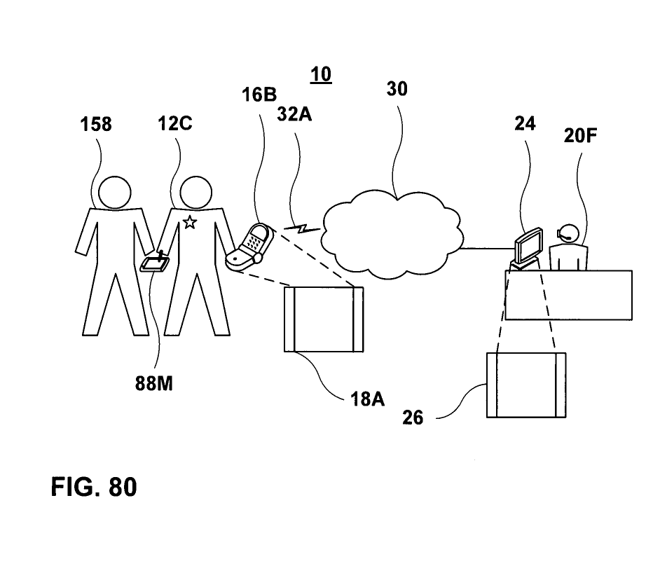

A further embodiment of the Remote Diagnostic & Treatment System 10 is shown in FIG. 80 in which a policeman 12D uses a remote fingerprint device 88M attached to a cellular or PCS wireless phone 16B to fingerprint a suspect 158. The fingerprint is transmitted to an analyst 20G for review and matching to fingerprint databases.

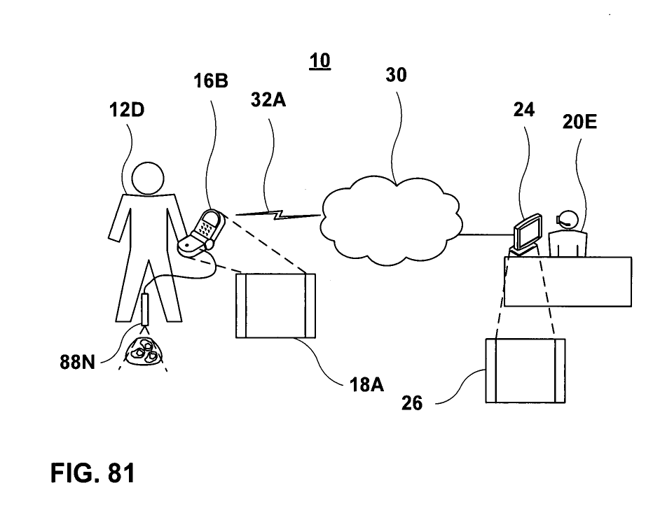

A further embodiment of the Remote Diagnostic & Treatment System 10 is shown in FIG. 81 in which an engineer 12E uses a soil sampling device 88N attached to a cellular or PCS wireless phone 16B. Characteristics of soil sample are transmitted to an laboratory technician 20F for assessment.

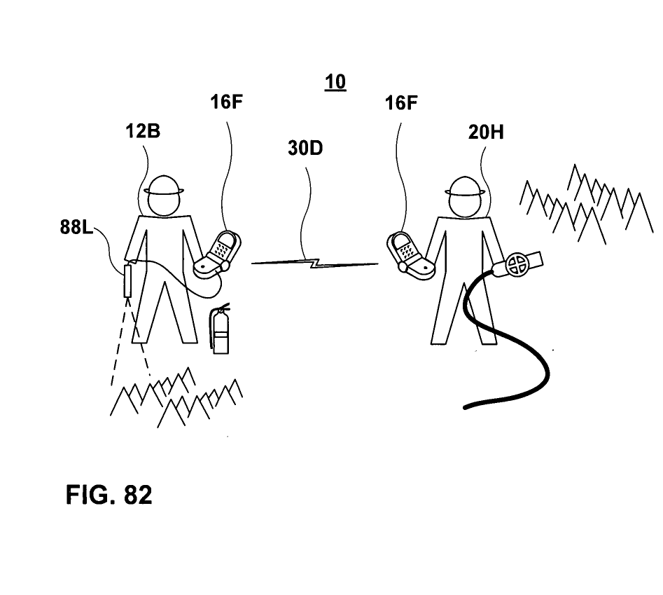

It is highly desirable in certain situations for remotely deployed personnel to share data in real time. FIG. 82 shows an embodiment of the Remote Diagnostic & Treatment System 10 in which a fire investigator 12C is using a chemical sniffer 88L attached to a walkie-talkie phone 16F and directly transmits 32D that data in real time to a fire fighter 20H actively fighting the fire nearby.

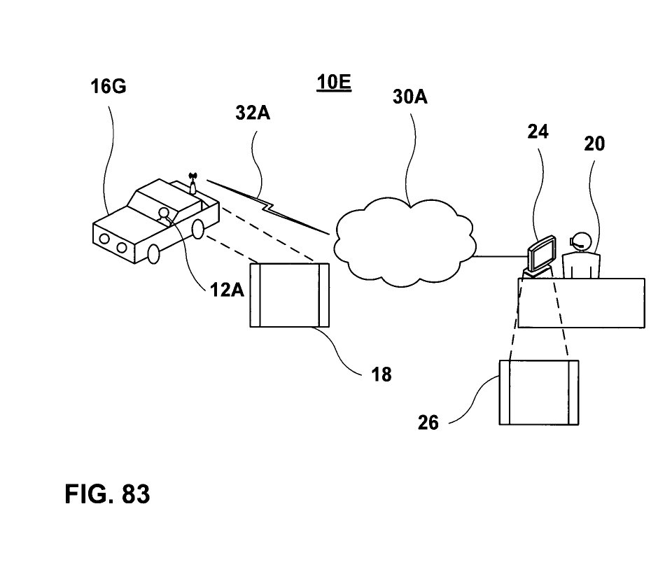

Many automobiles today include remote diagnostic and monitoring systems predominantly based upon cellular and PCS systems. Perhaps the best known such system is OnStar®, which is available in vehicles from General Motors Corporation (GM). An alternative embodiment of the Remote Diagnostic & Treatment System 10E utilizes OnStar® or similar systems 16G as a remote diagnostic means as shown in FIG. 83.

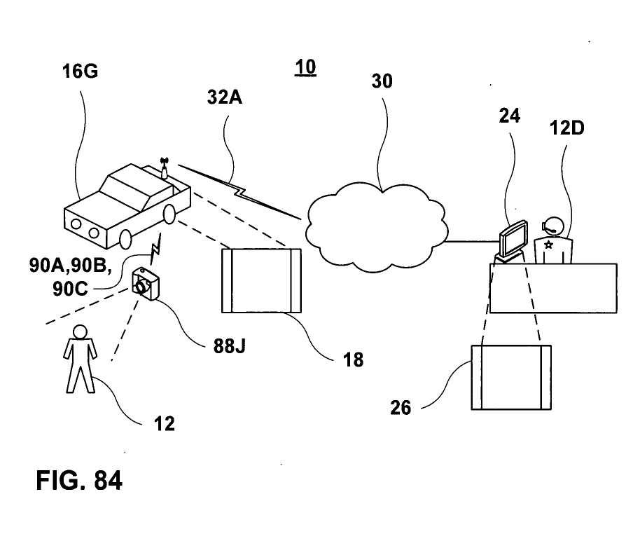

A further embodiment of the Remote Diagnostic & Treatment System 10E utilizing OnStar® or similar systems 16G is shown in FIG. 84. In this embodiment a camera 88J outside the car or mounted in the car is used to observe persons 12 outside the car. In the case of potential theft or an accident, images 42 and data may be transmitted to police 12D.

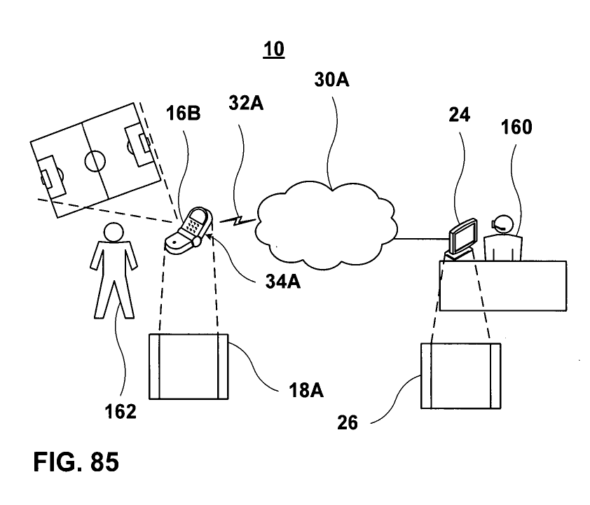

The Remote Diagnostic & Treatment System 10 may allow Grandpa 160 to watch the grandchildren playing soccer through Mom's 162 cellular or PCS wireless phone 16B camera 34A. While Mom 162 may hold the camera 34A, Grandpa 160 can control the view of the camera 34A to see what he wants to see, as shown in FIG. 85.

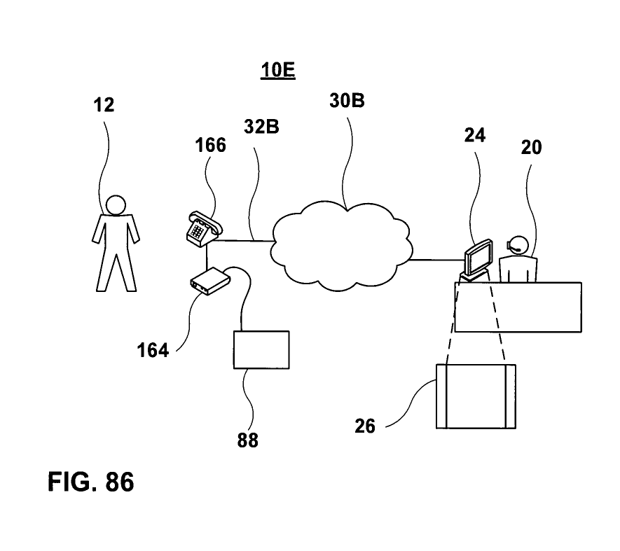

An alternative embodiment of the Remote Diagnostic & Treatment System 10 is shown in FIG. 86. In this embodiment data devices 88 are connected to a home diagnostic device 164 that is connected to a standard telephone 166 that is connected 32B to the PSTN 30B.

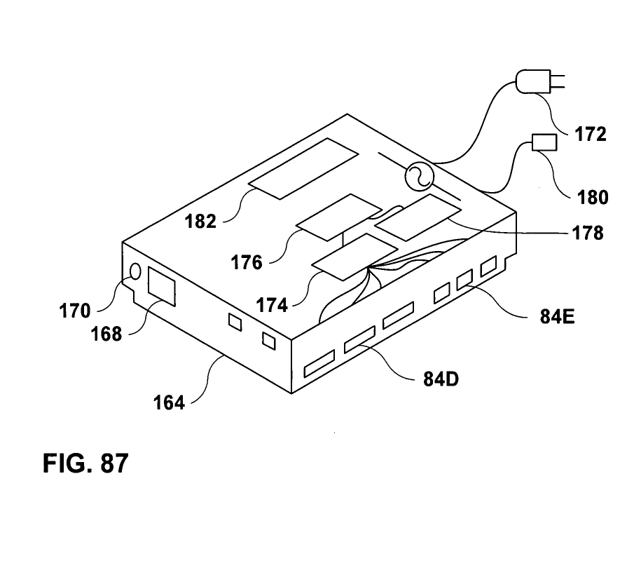

FIG. 87 shows a functional schematic of the home diagnostic device 164. The home diagnostic device 164 includes an “on” and “off” switch 168 and a Light Emitting Diode (LED) 170 that illuminates if the home diagnostic device 164 is receiving power from being plugged 172 into a household plug. Data devices 88 connect to the home diagnostic device 164 via USB 90D or FireWire 90E connections. Data from the connections are mixed in a data mixer 174, amplified by an amplifier 176 and fed to a modem 178. The modem 178 connects to the telephone 164 via an RJ11 connector 180. The home diagnostic device 164 includes battery backup 182 in case of power interruptions.

V. Purpose-Built Alternative Embodiment of a Diagnostic Means

The embodiments described thus far have been based upon modification and integration of predominantly existing technologies, products and devices. There are additional capabilities to those described above to be considered if starting with a blank sheet of paper to design a diagnostic means 16H.

In addition to a visible light camera 34A it may be desirable to have an infrared camera 34E that images the same injury or health condition; the infrared camera 34E basically “sees” heat that may be indicative of an elevated temperature. It is highly desirable to have a light 184 that can be remotely adjusted 46BC to provide the best possible illumination.

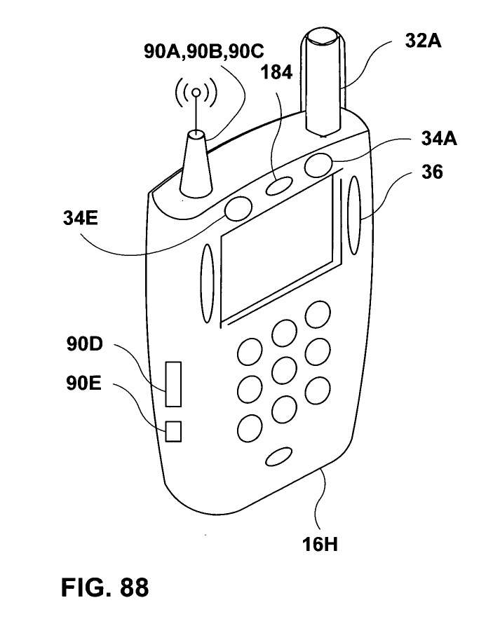

It is desirable that the designed diagnostic means 16H be able to communicate with data devices 88 via Bluetooth 90A, WiFi 90B, UWB 90C as well as other waveforms that might appear in the future. The designed diagnostic means 16H should be able to communicate with networks 30 using cellular or PCS 32A, WiFi 90B as well as other waveforms that might appear in the future. The designed diagnostic means 16H should have one or more USB 90D and one or more FireWire 90E ports built in as well as a speaker phone 36. An embodiment of a designed diagnostic means 16H is shown in FIG. 88.

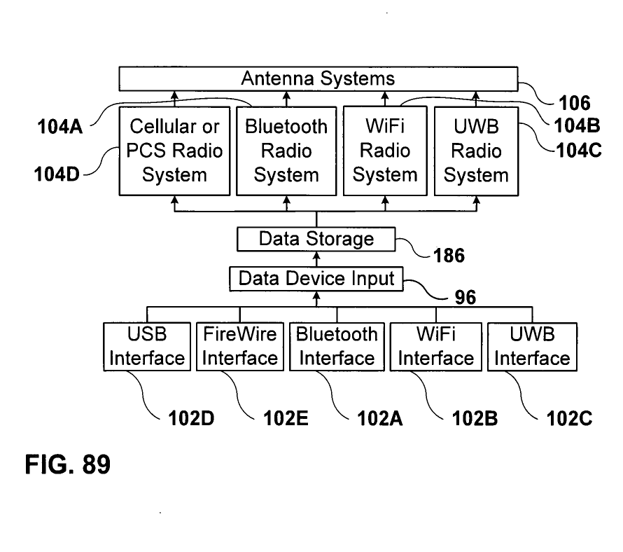

Today's cellular and PCS wireless 16B or WiFi 90B wireless phones have no or limited data storage capability for inputs from data devices 96. It is desirable to include data storage 186 in a designed diagnostic means 16H as shown in FIG. 89.

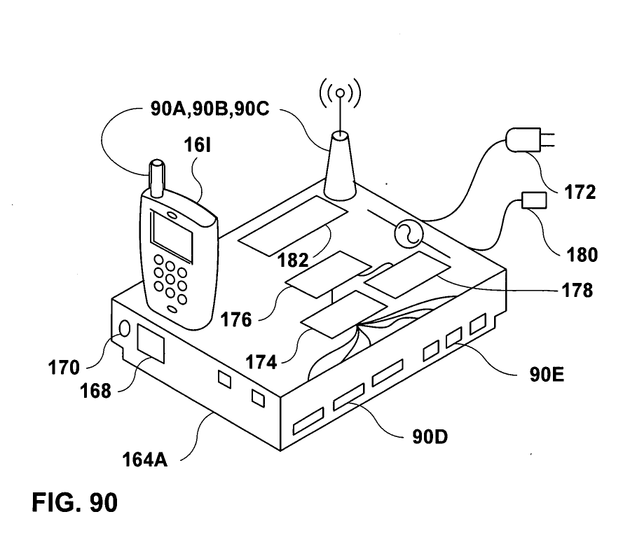

FIG. 90 shows an alternative embodiment of a designed diagnostic means 16I. In this embodiment the designed diagnostic means 16I sits in and is recharged by an embodiment of a home diagnostic device 164A. The designed diagnostic means 16I communicates with the home diagnostic device 164A via Bluetooth 90A, WiFi 90B, UWB 90C or other waveforms.

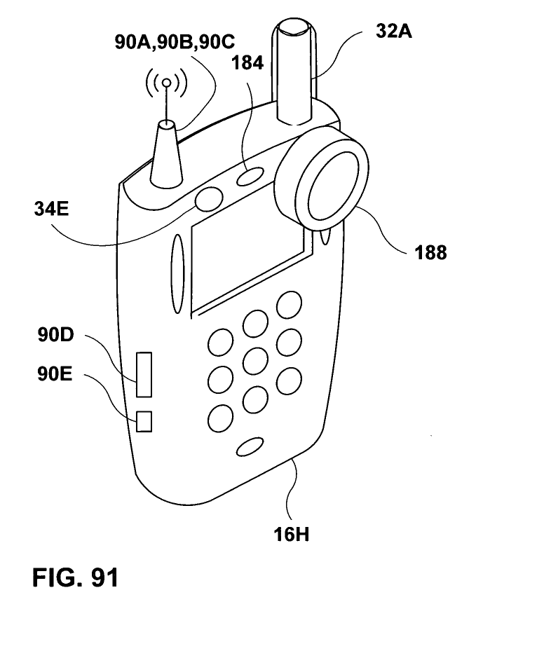

FIG. 91 shows an alternative embodiment of a designed diagnostic means 16H with a accessory macro lens 188 enabling a health service practitioner 20C to see or examine more detail of an injury or health condition.

VI. Remote Diagnostic and Health Status Monitoring Service

FIGS. 1 through 5, 57 through 68, 70 through 72, and 75 through 77 show embodiments of remote diagnostic means. FIGS. 67, 68 and 75 show embodiments of devices 138 that may be deployed on people in fixed locations or in a mobile environment. To provide remote diagnostic and health status monitoring services the remote diagnostic means 16A and the devices 138 need to connect to a health service practitioner 20C as shown in FIGS. 4 and 5, 12 through 14, 70 through 74, 76 and 77. The health service practitioner 20C must have a diagnostic display and control means 24A which runs one or more software application(s) 26A. There are a variety of institutional structures and business models under which such services may be provided.

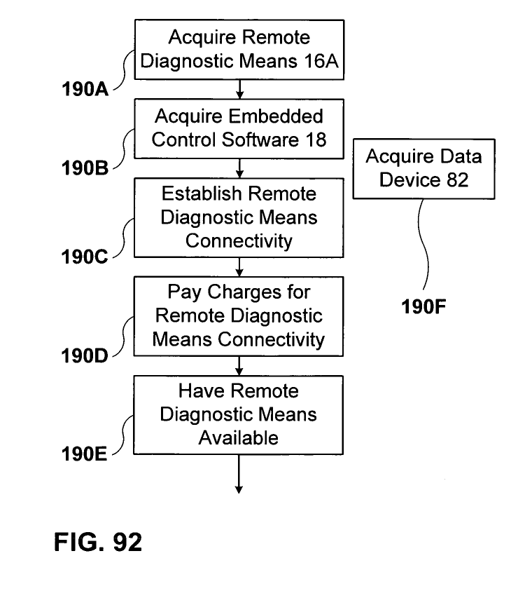

A patient or potential patient 12A must take certain steps 190 to receive remote diagnostic and health status monitoring services as shown in FIG. 92:
Acquire 190A remote diagnostic means 16A;
Acquire 190B embedded control software 18;
Establish 190C remote diagnostic means connectivity 30;
Pay charges for 190D for remote diagnostic means connectivity;
Have remote diagnostic means 16A available 190E.
   The patient or potential patient 12A may also elect to acquire 190F one or more data devices 88.

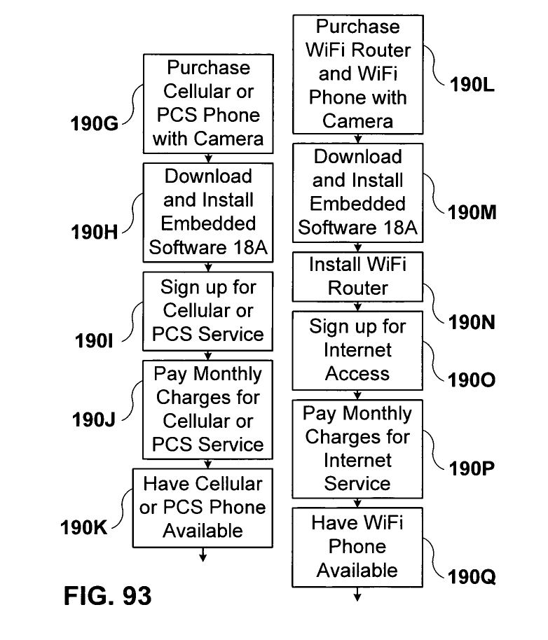

As shown in FIG. 93, if the patient or potential patient 12A plans to use a cellular or PCS wireless phone 16B as the remote diagnostic means 16A, he or she must:
Purchase a cellular or PCS wireless phone 16B that has a camera 34A in it 190G;
Download and install 190H diagnostic means software 18A to the cellular or PCS wireless phone 16B;
Sign up for cellular or PCS wireless service 190I;
Pay the monthly charges for the cellular or PCS wireless service 190J; and
Have the camera 34A equipped cellular or PCS wireless phone 16B available 190K in case of an emergency or for routine health status monitoring.
   If the patient or potential patient 12A plans to use a WiFi wireless phone 16J as the remote diagnostic means 16A, he or she must:
Purchase a WiFi wireless phone 16J that has a camera 34F in it and a WiFi router 190L;
Download and install 190M diagnostic means software 18D to the WiFi wireless phone 16J;
Install 190N the WiFi router 192;
Sign up for Internet access 190O;
Pay the monthly charges for Internet access 190P; and
Have the camera 34F equipped WiFi wireless phone 16J available 190Q in case of an emergency or for routine health status monitoring.
   If the patient or potential patient 12A plans to use the device 138 shown in FIGS. 67 and 68 as the remote diagnostic means 16A, he or she must follow the steps 188 described above depending upon whether the device 138 includes a cellular or PCS wireless phone 16B or a WiFi phone 16J.

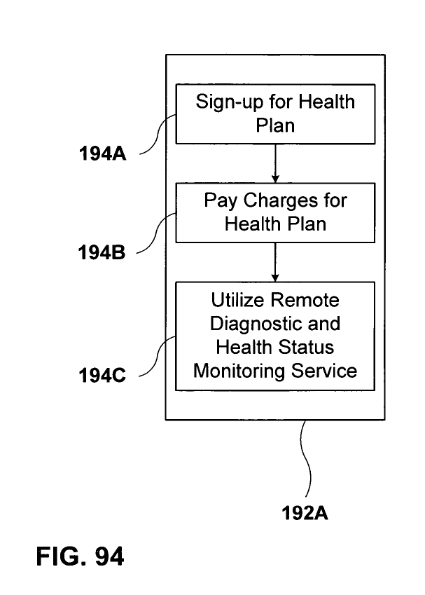

A functional block diagram 194A of a first embodiment of an institutional structure or business model under which remote diagnostic and health status monitoring services may be provided is shown in FIG. 92. In this embodiment the patient or potential patient 12A takes the steps 196 of:
Signing up 196A for health plan coverage;
Paying the fees 196B for health plan coverage;
Utilizing remote diagnostic and health status monitoring services 196C.
   In the embodiment shown in FIG. 94 the health plan makes remote diagnostics and health status monitoring available as part of its health plan. In the embodiment shown in FIG. 94 remote diagnostics and health status monitoring are provided as part of the health plan fee.

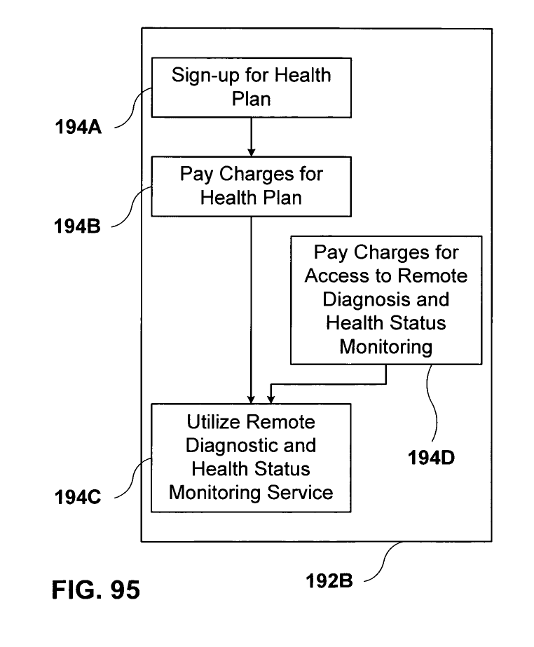

In a second embodiment of a functional block diagram of an institutional structure or business model under which remote diagnostic and health status monitoring services may be provided 192B, as shown in FIG. 95, there is a charge for using remote diagnostics and health status monitoring services 194D even though the patient or potential patient 12A is covered by a health plan.

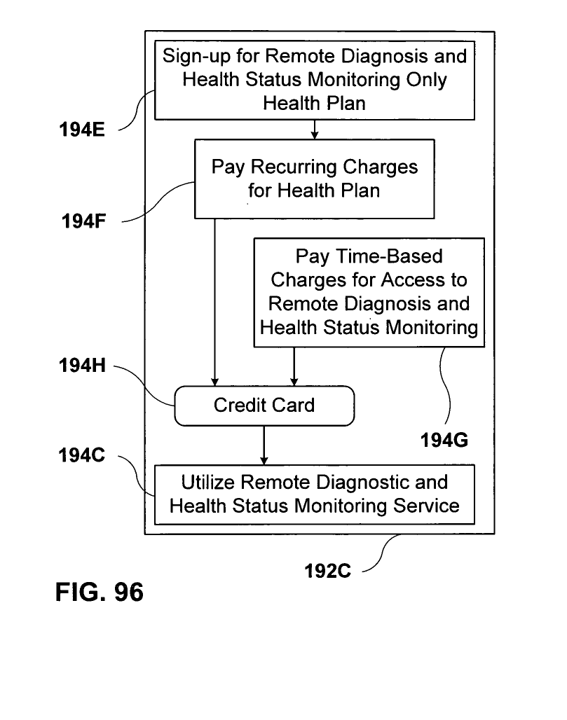

A large number of people do not have any health insurance, are under insured meaning they do not have enough medical insurance for their situation, or are self insured meaning they pay out of their pocket for health or medical services. These people, and others, may utilize a remote diagnostic and health status monitoring service if the charges are appropriate. A third embodiment of a functional block diagram of an institutional structure or business model under which remote diagnostic and health status monitoring services may be provided 192C is shown in FIG. 96. In this embodiment the patient or potential patient 12A signs up for a remote diagnostic and health status monitoring only health plan 194E; pays a nominal regular fee 194F, for example, Ten Dollars ($10.00) per month; and pays a time-based fee 194G for using such remote diagnostic and health status monitoring service, for example, Two Dollars ($2.00) per minute, which fees may be paid using a credit card 194H.

VII. Internet Doctor

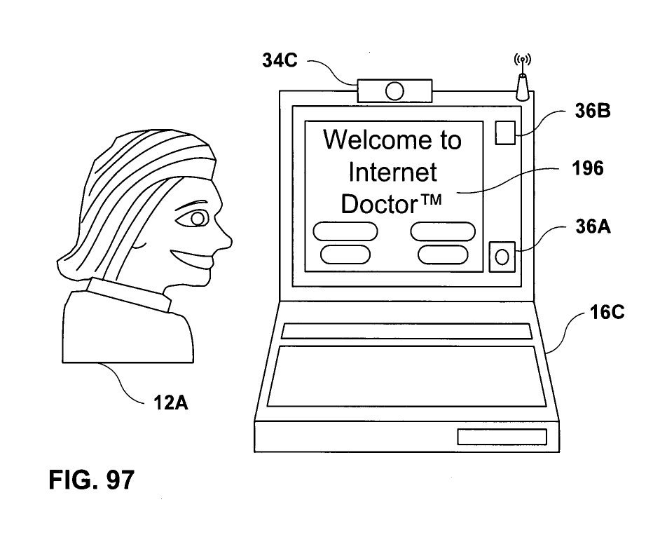

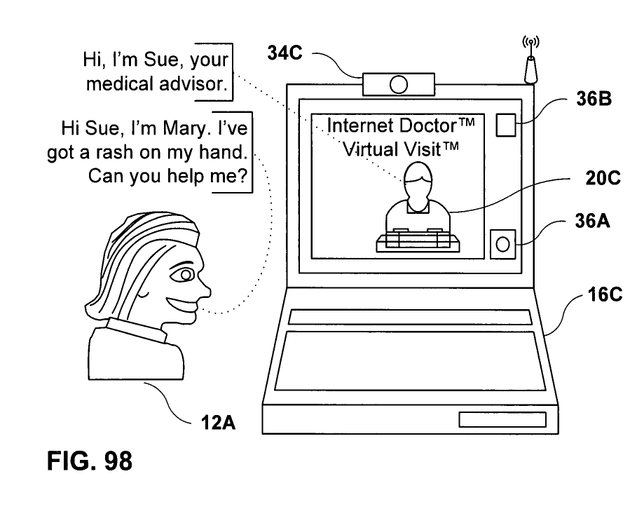

In another embodiment, the invention is employed to provide medical information and advice using a website, such as www.InternetDoctor.com. FIG. 97 shows a patient or potential patient 12A who utilizes a personal computer 16C to visit a website 198 to obtain medical information and/or advice. In FIG. 98, the patient or potential patient 12A requests information about a rash on her hand.

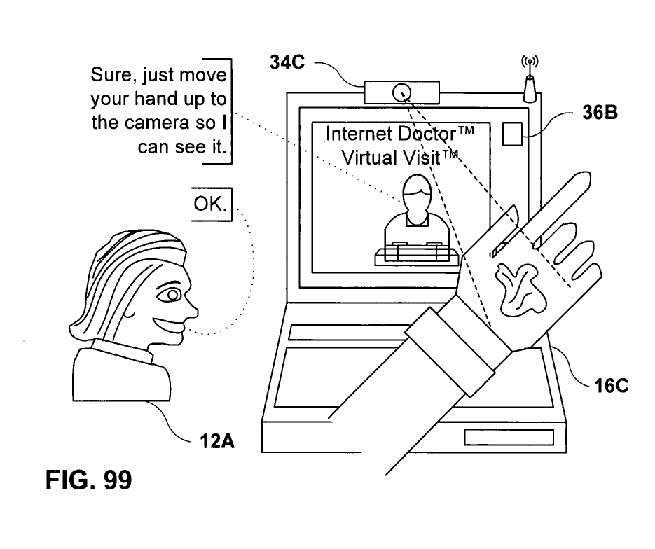

FIG. 99 illustrates the patient or potential patient 12A placing her hand near the camera 34C of the personal computer 16C, which allows the health service practitioner 20C to see the rash on a remote display.

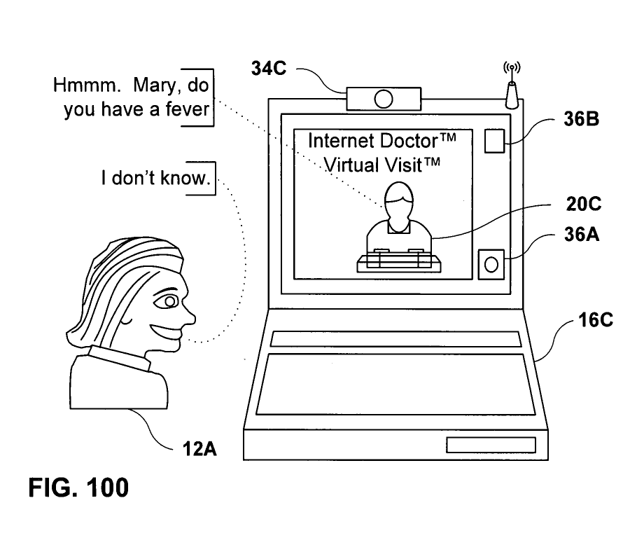

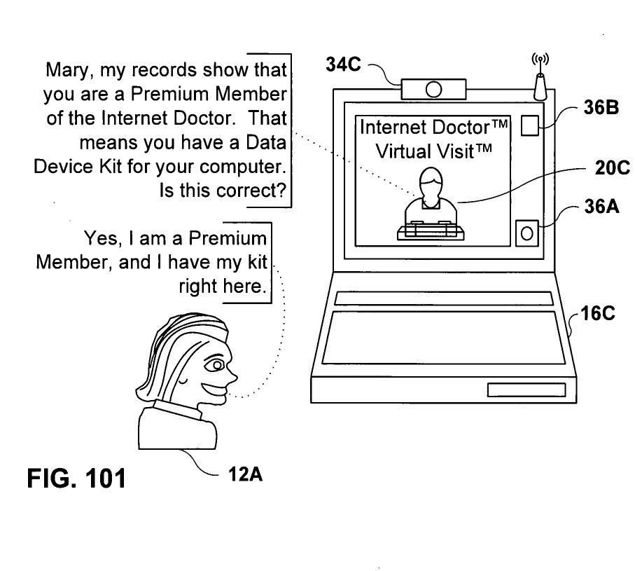

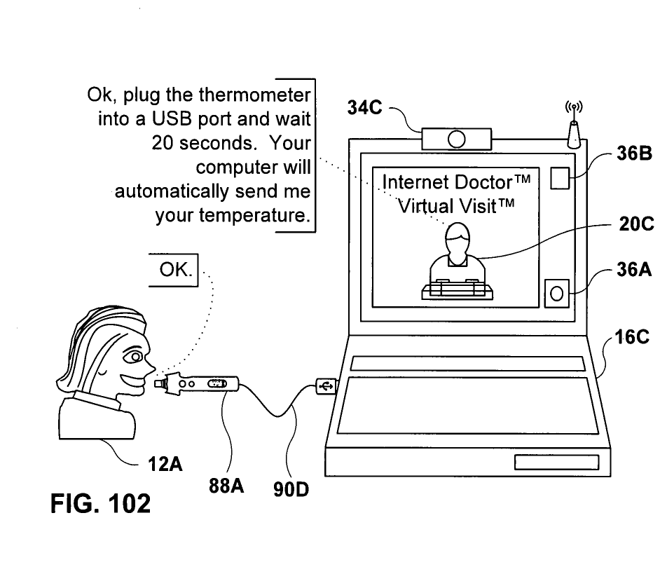

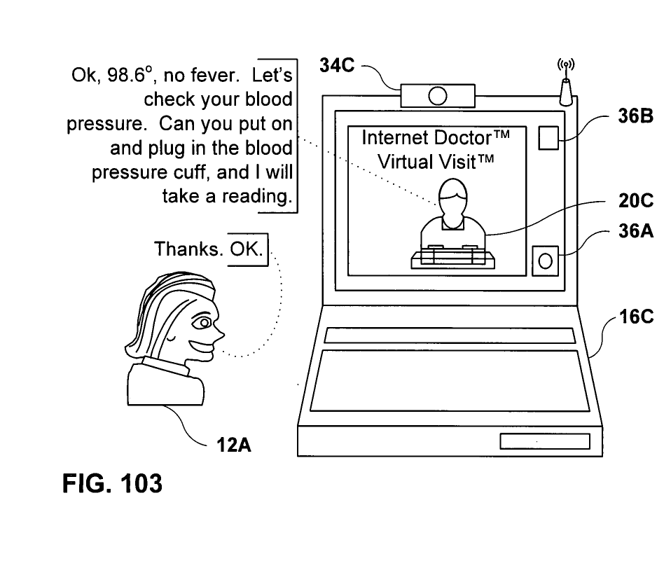

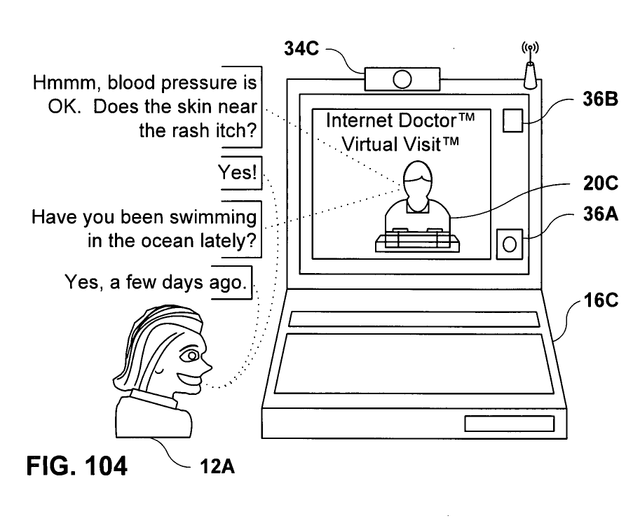

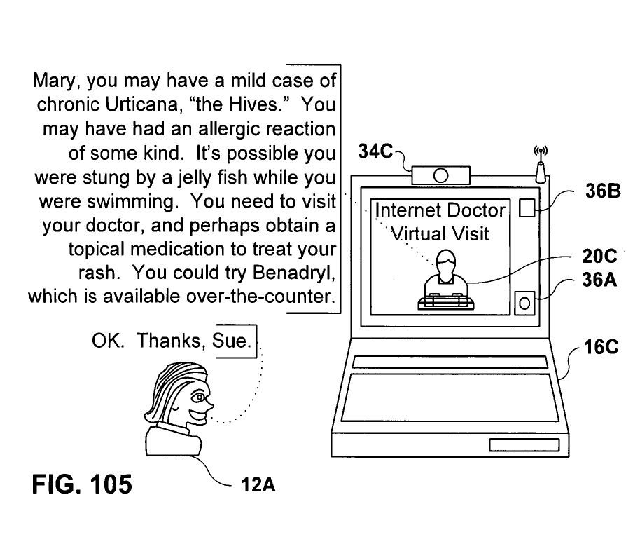

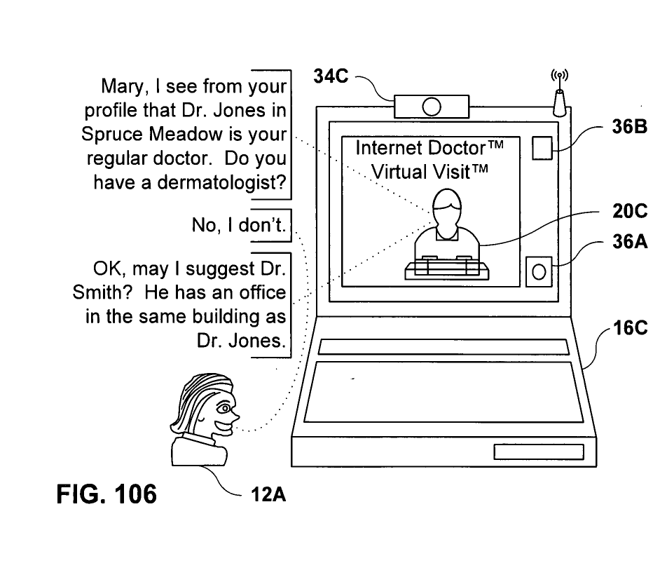

FIGS. 100 and 101 reveal additional dialog between the patient or potential patient 12A and the health service practitioner 20C, which enables the health service practitioner 20C to provide a remote diagnosis. FIGS. 102 through 104 portray the use of a data devices 88, which are connected to the personal computer to enable the patient or potential patient 12A to send medical data to the advisor through the personal computer 16C and then over the Internet 30I. FIGS. 105 and 106 provide views of the health service practitioner 20C offering a preliminary diagnosis.

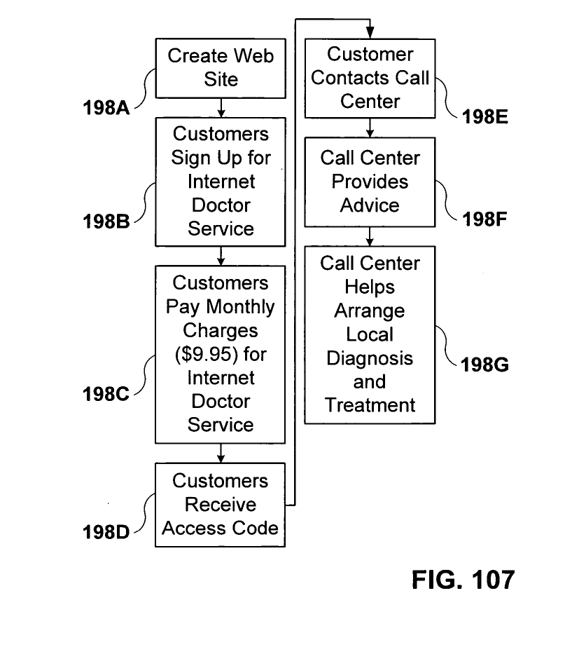

FIG. 107 is a flow chart which exhibits one method of the present invention. In the first step, a website is created 198A and is made available over the Internet. Patients or potential patients 12A then sign up for the “Internet Doctor”™ Service 198B. These patients or potential patients 12A pay a fee 198C, such as a monthly fee of $9.95. Each patient or potential patient 12A receives an access code 198D that enables him or her to use the service.

When a patient or potential patient 12A needs information, he or she places a call 198E to a cell center 28 using a wired or wireless phone. A health service practitioner 20C at the call center 28 provides information during the call 198F, and may help to arrange local diagnosis and treatment 198G for the patient or potential patient 12A.

### Conclusion

Although the present invention has been described in detail with reference to one or more preferred embodiments, persons possessing ordinary skill in the art to which this invention pertains will appreciate that various modifications and enhancements may be made without departing from the spirit and scope of the Claims that follow. The various alternatives that have been disclosed above are intended to educate the reader about preferred embodiments of the invention, and are not intended to constrain the limits of the invention or the scope of Claims.

### List of Reference Characters

10 Remote Diagnostic & Treatment System
12 First person or user
12A Patient or potential patient
12B Animal
12C Fire investigator
12D Policeman
12E Engineer
14 First person location
16 First device or terminal
16A Remote diagnostic means
16B Cellular or Personal Communications Service (PCS) wireless phone
16C Laptop personal computer (PC)
16D Desktop personal computer (PC)
16E Satellite phone
16F Walkie-talkie phone
16G OnStar® phone
16H Purpose-built diagnostic means
16I Alternative purpose-built diagnostic means
16J Wireless fidelity (WiFi) wireless phone
18 Embedded software that enables remote functional control of the first person device or terminal
18A Embedded software that enables remote functional control of a cellular or Personal Communications Service (PCS) wireless phone
18B Embedded software that enables remote functional control of a laptop personal computer used as a diagnostic means
18C Embedded software that enables remote functional control of a desktop personal computer used as a diagnostic means
18D Embedded software that enables remote functional control of a Wireless Fidelity (WiFi) phone used as a diagnostic means
20 Second person or user
20A Call center live operator
20B Single person offering assistance
20C Health service practitioner
20D Physical therapist or athletic coach
20E Veterinarian
20F Laboratory technician
20G Fingerprint analyst
20H Firefighter
22 Second person location
24 Second device or terminal
24A Diagnostic, display and control means
26 Embedded software that enables assistance to a first person and enables remote functional control of the first person device or terminal
26A Diagnostic, display and control software application for remotely controlling a cellular or Personal Communications Service (PCS) wireless phone
26B Diagnostic, display and control software application for remotely controlling a laptop or desktop personal computer
28 Call center
30 Network
30A Cellular or Personal Communications Service (PCS) network
30B Public Switched Telephone Network (PSTN)
30C Satellite network
30I Internet
32 Connection to a network
32A Connection to a cellular or Personal Communications Service (PCS) network
32B Connection to a Public Switched Telephone Network (PSTN)
32C Connection to a satellite network
32D Walkie-talkie connection
32I Connection to the Internet
34 Camera
34A Camera in a cellular or Personal Communications Service (PCS) wireless phone
34B Camera in an otoscope
34C Camera in a laptop personal computer
34D Camera attached to a desktop personal computer
34E Infrared camera
34F Camera in a Wireless Fidelity (WiFi) wireless phone
36 Speaker phone
36A Speaker
36B Microphone
36C Speaker phone in a cellular or Personal Communications Service (PCS) wireless phone
38 Camera image
40 Device or terminal display screen
44A Display screen in a cellular or Personal Communications Service (PCS) wireless phone
42 Picture
44 Functional block diagram of embedded software that enables remote functional control of the diagnostic means
44A Functional block diagram of embedded software that enables remote functional control of a cellular or Personal Communications Service (PCS) wireless phone
46 Diagnostic means functions to be controlled remotely
46A Turn a camera on or off
46B Change a camera image size
46C Adjust a camera focus
46D Adjust a camera color
46E Adjust a camera hue
46F Adjust a camera contrast
46G Take a picture
46H Turn a speaker phone on or off
46I Enter a phone number to which to send a picture
46J Enter an electronic mail address to which to send a picture
46K Turn a thermometer on or off
46L Capture and send a temperature reading
46M Turn the temperature reading display of the cellular and PCS wireless phone on or off
46N Turn a stethoscope or high fidelity microphone on or off
46O Adjust the volume of a stethoscope or high fidelity microphone
46P Adjust the tone of a stethoscope or high fidelity microphone using an equalizer
46Q Capture and send a pulse reading
46R Turn a scale on or off
46S Capture and send a weight reading
46T Turn a weight reading display on or off
46U Inflate or deflate a blood pressure cuff
46V Capture and send a blood pressure reading
46W Turn the blood pressure and pulse readings display on or off
46X Turn an oximeter on or off;
46Y Capture and send a reading of the percent of hemoglobin that is saturated with oxygen
46Z Turn a reading of the percent of hemoglobin that is saturated with oxygen and pulse reading display on or off
46AA Turn an electrocardiogram unit on or off
46AB Capture and send an electrocardiogram reading
46AC Turn the electrocardiogram reading display on or off.
46AD Turn a glucose meter on or off
46AE Capture and send a glucose reading
46AF Turn a glucose reading display on or off
46AG Turn a camera in an otoscope on or off
46AH Zoom a camera in an otoscope
46AI Focus the camera in an otoscope
46AJ Adjust the camera color in an otoscope
46AK Adjust the camera hue in an otoscope
46AL Adjust the camera contrast in an otoscope
46AM Take a picture with the camera in an otoscope
46AN Turn the otoscope camera display on or off
46AO Turn an ultrasound unit on or off
46AP Capture and send an ultrasound reading
46AQ Turn the ultrasound display on or off
46AR Turn an external camera on or off
46AS Zoom an external camera
46AT Focus an external camera
46AU Pan an external camera
46AV Tilt an external camera
46AW Adjust the color of an external camera
46AX Adjust the hue of an external camera
46AY Adjust the contrast of an external camera
46AZ Take a picture with an external camera
46BA Take a video with an external camera
46BB Turn an external camera display on or off
46BC Control a light
48 Telephone number
50 Electronic mail address
52 First screen of a software application that enables remote functional control of the diagnostic means
54 Incoming phone number
56 Alternate phone number
58 Patient or potential patient name
60 Patient or potential patient date of birth
62 Patient or potential patient Social Security Number (SSN)
64 Health insurance carrier
66 Pull-down menu
68 Other patient information
70 Note area
72 Date of phone call
74 Current time
76 Start time and stop time of phone call
78 Phone call duration
80 Functional block diagram of a software application that enables remote functional control of the diagnostic means
80A Functional block diagram of a software application that enables remote functional control of a cellular or Personal Communications Service (PCS) wireless phone
82 Functional control means
82A “Off” button and an “on” button for controlling a camera
82B Slider that “zooms” a camera image size from zero percent (0%) to one hundred percent (100%)
82C Slider that adjusts a camera focus from minus (−) to plus (+)
82D Slider that adjusts the color from zero percent (0%) to one hundred percent (100%)
82E Slider that adjusts the hue from zero percent (0%) to one hundred percent (100%)
82F Slider that adjusts the contrast from minus (−) to plus (+)
82G Button for taking a picture
82H “Off” button and an “on” button for controlling a speaker phone
82I Button for dialing a phone number to which the picture is to be sent
82J Button for sending a picture to an electronic mail address
82K “Off” button and an “on” button for controlling a thermometer
82L Button for capturing or recapturing a temperature reading
82M Button for sending a temperature reading to a health service practitioner
82N “Off” button and an “on” button for controlling the display of a temperature reading on a cellular and PCS wireless phone
82O Save a temperature reading to a patient's or potential patient's electronic medical file.
82P “Off” button and an “on” button for controlling a stethoscope or high fidelity microphone
82Q Slider that adjusts the volume of a stethoscope or high fidelity microphone from minus (−) to plus (+)
82R Multiple sliders that adjust the tone of a stethoscope or high fidelity microphone from minus (−) to plus (+)
82S Button for capturing or recapturing the pulse reading
82T Button for sending the pulse reading from a stethoscope or high fidelity microphone to a health service practitioner
82U Save the pulse reading from a stethoscope or high fidelity microphone to the patient's or potential patient's electronic medical file
82V “Off” button and an “on” button for controlling a scale
82W Button for capturing or recapturing a weight reading
82X Button for sending a weight reading to a health service practitioner
82Y “Off” button and an “on” button for controlling the display of a weight reading
82Z Save a weight reading to a patient's or potential patient's electronic medical file
82AA Button for inflating and deflating a blood pressure cuff
82AB Button for capturing or recapturing a blood pressure reading
82AC Button for sending the blood pressure reading to a health service practitioner
82AD “Off” button and an “on” button for controlling the display of blood pressure and pulse readings
82AE Save a blood pressure reading to a patient's or potential patient's electronic medical file
82AF “off” button and an “on” button for controlling an oximeter
82AG Button for capturing or recapturing a reading of the percent of hemoglobin that is saturated with oxygen
82AH Button for sending a reading of the percent of hemoglobin that is saturated with oxygen to a health service practitioner
82AI “Off” button and an “on” button for controlling a display of the reading of the percent of hemoglobin that is saturated with oxygen and pulse readings
82AJ Save a reading of the percent of hemoglobin that is saturated with oxygen to the patient's or potential patient's electronic medical file
82AK “Off” button and an “on” button for controlling an electrocardiogram unit
82AL Button for capturing or recapturing an electrocardiogram reading
82AM Button for sending an electrocardiogram reading to a health service practitioner
82AN “Off” button and an “on” button for controlling a display of the electrocardiogram
82AO Save an electrocardiogram reading to a patient's or potential patient's electronic medical file
82AP “Off” button and an “on” button for controlling a glucose meter
82AQ Button for capturing or recapturing a glucose reading
82AR Button for sending a glucose reading to a health service practitioner
82AS “Off” button and an “on” button for controlling the display of a glucose reading
82AT Save a glucose reading to a patient's or potential patient's electronic medical file
82AU “Off” button and an “on” button for controlling the camera in an otoscope
82AV Slider that adjusts the zoom of a camera in an otoscope from zero percent (0%) to one hundred percent (100%)
82AW Slider that adjusts the camera focus in an otoscope from minus (−) to plus (+)
82AX Slider that adjusts the color of a camera in an otoscope from zero percent (0%) to one hundred percent (100%)
82AY Slider that adjusts the hue of a camera in an otoscope from zero percent (0%) to one hundred percent (100%)
82AZ Slider that adjusts the contrast of a camera in an otoscope from minus (−) to plus (+)
82BA Button for taking a picture with a camera in an otoscope
82BB Button for sending the picture taken with a camera in an otoscope to a health service practitioner
82BC “Off” button and an “on” button for controlling the display of a camera in an otoscope
82BD Save the picture from a camera in an otoscope to a patient's or potential patient's electronic medical file
82BE “Off” button and an “on” button for controlling an ultrasound unit
82BF Button for capturing or recapturing an ultrasound readings
82BG Button for sending ultrasound readings to a health service practitioner
82BH “Off” button and an “on” button for controlling the ultrasound display
82BI Save ultrasound readings to a patient's or potential patient's electronic medical file
82BJ “Off” button and an “on” button for controlling an external camera
82BK Slider that adjusts the zoom of an external camera from zero percent (0%) to one hundred percent (100%)
82BL Slider that adjusts the focus of an external camera from minus (−) to plus (+)
82BM Slider that adjusts the pan of an external camera from minus (−) to plus (+)
82BN Slider that adjusts the tilt of an external camera from minus (−) to plus (+)
82BO Slider that adjusts the color of an external camera from zero percent (0%) to one hundred percent (100%)
82BP Slider that adjusts the hue of an external camera from zero percent (0%) to one hundred percent (100%)
82BQ Slider that adjusts the contrast of an external camera from minus (−) to plus (+)
82BR Button for taking a picture with an external camera
82BS Button for sending a picture taken with an external camera to a health service practitioner
82BT Button for capturing video with an external camera
82BU Button for sending a video taken with an external camera to a health service practitioner
82BV “Off” button and an “on” button for controlling the external camera display
82BW Save a picture taken with an external camera to a patient's or potential patient's electronic medical file
82BX Save video taken with an external camera to a patient's or potential patient's electronic medical file
84 Screen displayed on a diagnostic, display and control means
86 Picture window on a diagnostic, display and control means
88 Data devices
88A Digital thermometer
88B Stethoscope or high fidelity microphone
88C Weight scale
88D Blood pressure cuff
88E Oximeter
88F Electrocardiogram (EKG)
88G Glucose meter
88H Otoscope
88I Ultrasound device
88J External camera
88K Heart rate monitor
90 Connection between a data device and a diagnostic means
90A Bluetooth® wireless link
90B Wireless fidelity (WiFi) wireless local area network link, Institute of Electrical and Electronic Engineers (IEEE) 802.11
90C Ultra Wide Band (UWB) connection
90D Universal Serial Bus (USB) connection
90E FireWire connection, Institute of Electrical and Electronic Engineers (IEEE) 1394
92 Connection interface device
94 Functional block diagram of connection interface device
96 Data device input to connection interface device
98 Preamplifier in connection interface device
100 Amplifier in connection interface device
102 Connection interface in connection interface device
102A Bluetooth® interface in connection interface device
102B Wireless fidelity (WiFi) interface in connection interface device
102C Ultra Wide Band (UWB) interface in connection interface device
102D Universal Serial Bus (USB) interface in connection interface device
102E FireWire interface in connection interface device
104 Radio system
104A Bluetooth® radio system in connection interface device
104B Wireless fidelity (WiFi) radio system in connection interface device
104C Ultra Wide Band (UWB) radio system in connection interface device
104D Cellular or Personal Communications Service (PCS) radio system in connection interface device
106 Antenna systems
108 Functional block diagram of embedded software that enables remote functional control of data devices for the diagnostic means as well as the corresponding software application that enables remote functional control of the data devices for the diagnostic means
108A Functional block diagram of software embedded in a diagnostic means that enables remote functional control of data devices via the diagnostic means
108B Software application deploy on the diagnostic, display and control means that enables remote functional control of the data devices via the diagnostic means
110 Temperature pop-up window
112 Temperature location on body
112A Temperature taken in the ear
112B Temperature taken on the ear lobe
112C Temperature taken rectally
112D Temperature taken in other location
114 “Done” button
116 Temperature window on screen displayed on a diagnostic, display and control means
118 Pulse window on screen displayed on a diagnostic, display and control means
120 Transducer for converting stethoscope sounds to electrical signals
122 Weight window on screen displayed on a diagnostic, display and control means
124 Blood pressure window on screen displayed on a diagnostic, display and control means
126 Oximeter window on screen displayed on a diagnostic, display and control means
128 Electrocardiogram window on screen displayed on a diagnostic, display and control means
130 Glucose window on screen displayed on a diagnostic, display and control means
132 Otoscope (picture) window on screen displayed on a diagnostic, display and control means
134 Ultrasound (video) window on screen displayed on a diagnostic, display and control means
136 Video
138 Health status monitoring device
140 Watch
142 Emergency call button
144 Global Positioning System (GPS)
146 Global Positioning System (GPS) window on screen displayed on a diagnostic, display and control means
148 Stop watch
150 Ship
152 Satellite
154 Earth station
156 Airplane
158 Police suspect
160 Grandparent
162 Mother
164 Home diagnostic device
164A Alternative home diagnostic device
166 Telephone
168 “On” and “off” switch for home diagnostic device
170 Power “on” Light Emitting Diode (LED)
172 Household power plug
174 Data mixer
176 Amplifier
178 Modem
180 RJ11 connector
182 Battery backup
184 Light
186 Data storage
188 Macro lens
190 Steps that must be taken by a patient or potential patient to receive remote diagnostic or health status monitoring services
190A Acquire remote diagnostic means
190B Acquire embedded control software for remote diagnostic means
190C Establish remote diagnostic means connectivity
190D Pay charges for remote diagnostic means connectivity
190E Have remote diagnostic means available
190F Acquire one or more data devices
190G Purchase a cellular or Personal Communications Service (PCS) wireless phone that includes a camera
190H Download and install diagnostic means software to a cellular or Personal Communications Service (PCS) wireless phone
190I Sign up for cellular or Personal Communications Service (PCS) wireless service
190J Pay the monthly charges for the cellular or Personal Communications Service (PCS) wireless service
190K Have the camera-equipped cellular or Personal Communications Service (PCS) wireless phone available in case of an emergency or for routine health status monitoring
190L Purchase a Wireless Fidelity (WiFi) wireless phone that has a camera in it and a WiFi router
190M Download and install diagnostic means software to a Wireless Fidelity (WiFi) wireless phone
190N Install a Wireless Fidelity (WiFi) router
190O Sign up for Internet access
190P Pay the monthly charges for Internet access
190Q Have the camera-equipped Wireless Fidelity (WiFi) wireless phone available in case of an emergency or for routine health status monitoring
192 Wireless router
194 Functional block diagram of an institutional structure or business model under which remote diagnostic and health status monitoring services may be provided
194A Functional block diagram of an institutional structure or business model under which remote diagnostic and health status monitoring services are provided as part of a health insurance plan
194B Functional block diagram of an institutional structure or business model under which remote diagnostic and health status monitoring services are provided for an additional fee as part of a health insurance plan
194C Functional block diagram of an institutional structure or business model under which remote diagnostic and health status monitoring services are provided for a recurring fee and a time-based utilization fee.
196 Steps a patient or potential patient takes to get remote diagnostic and health status monitoring services
196A Sign up for health plan coverage
196B Pay the fees for health plan coverage
196C Utilize remote diagnostic and health status monitoring services
196D Charge for using remote diagnostics and health status monitoring services
196E Sign up for a remote diagnostic and health status monitoring only health plan
196F Pays a recurring fee
196G Pay a time-based fee for using such remote diagnostic and health status monitoring service
196H Credit card payment
198 Elements of business model for Internet Doctor
198A Create Web site
198B Customers sign up for Internet Doctor service
198C Customers pay monthly charges for Internet Doctor service
198D Customers receive access code
198E Customer contacts call center
198F Call center provides diagnosis and advice
198G Call center provides local diagnosis and treatment options

What is claimed is:

1. An apparatus comprising:
a cellular telephone (16A);
said cellular telephone (16A) including a camera (34), a display (40), a speaker(36A), a microphone (36B), and a diagnostic software program (18);
a call center (28) operated by a call center operator (20A);
said call center (28) for receiving a telephone call from said user (12) of said cellular telephone (16A);
said call center operator (20A) for providing advice concerning said user (12) and for partially controlling said cellular telephone (16A).

2. An apparatus as recited in claim 1, in which said cellular telephone (16A) is used to provide triage for a patient (12A).

3. An apparatus as recited in claim 1, in which said cellular telephone (16A) is used for health monitoring.

4. An apparatus as recited in claim 1, further comprising a data device (88).

5. An apparatus as recited in claim 1, in which said data device (88) is connected to said cellular telephone (16A) by a wireless connection (30A).

6. An apparatus as recited in claim 5, in which said wireless connection (30A) is a Bluetooth® connection (84A).

7. An apparatus as recited in claim 5, in which said wireless connection (30A) is a Wi-Fi connection (84B).

8. An apparatus as recited in claim 5, in which said wireless connection (30A) is an ultra-wide band connection (84C).

9. An apparatus as recited in claim 1, in which said data device (88) is connected to said cellular telephone (16A) by a wired connection (30B).

10. An apparatus as recited in claim 1, in which said wired connection (30B) uses a USB connector (84D).

11. An apparatus as recited in claim 1, in which said wired connection (30B) uses a FireWire connector (84E).

12. An apparatus as recited in claim 4, in which said data device (88) is controlled remotely by said call center operator (20A).

13. An apparatus as recited in claim 4, in which said data device (88) is a thermometer (82A).

14. An apparatus as recited in claim 4, in which said data device (88) is an acoustic sensor (88B).

15. An apparatus as recited in claim 4, in which said data device (88) is a weight measurement device (88C).

16. An apparatus as recited in claim 4, in which said data device (88) is a blood pressure measurement device (88D).

17. An apparatus as recited in claim 4, in which said data device (88) is a device (88E) which measures the level of oxygen in the blood.

18. An apparatus as recited in claim 4, in which said data device (88) is a device for obtaining an electrocardiograph (88F).

19. An apparatus as recited in claim 4, in which said data device (88) is a glucose measurement device (88G).

20. An apparatus as recited in claim 4, in which said data device (88) is an otoscope (88H).

21. An apparatus as recited in claim 4, in which said data device (88) is an ultrasound imaging device (88I).

23. An apparatus as recited in claim 4, in which said data device (88) is an external camera (88J).

24. An apparatus as recited in claim 1, in which said cellular telephone (16A) is used for conveying information in a veterinary practice.

25. A method comprising the steps of:
using a diagnostic means (16A) for collecting a set of data;
said diagnostic means (16A) for collecting said set of data including an embedded software application (18); and
conveying said set of data from said diagnostic means (16A) to a diagnostic display and control means (24);
said diagnostic display and control means (24) including a software application (26).

26. A method as recited in claim 25, further comprising the step of:
controlling said diagnostic means (16A) using said diagnostic display and control means (24).

27. A method comprising the steps of:
providing a website; said website including a database of medical information;
offering a service which furnishes a user (12) with an access code; said access code enabling said user to utilize said database of medical information on said website;
calling a call center (28); said call center (28) being staffed by a health service practitioner (20C); and
supplying interactive advice to said user from said call center (28).

28. A method as recited in claim 27, further comprising the step of:
using a cellular telephone (16A) to provide information to said call center (28); said cellular telephone (16A) including a microphone (36B), a speaker (36A) and a camera (34).

29. A method as recited in claim 27, further comprising the step of:
providing data to said call center (28) using a data device (88); said data device (88) being connected to said cellular telephone (16A).

30. A method as recited in claim 1, in which said data device (88) is partially controlled by said call center (28).
